(19) United States
(12) Patent Application Publication
(10) Pub. No.: US 2009/0069315 A1
(43) Pub. Date: Mar. 12, 2009

(54) Use of Pyrazolo(1,5A)Pyrimidin-7-YL Amine Derivatives in the Treatment of Neurological Disorders

(76) Inventors: Rajeev Sivasankaran, Alton, MA (US); Kaspar Zimmermann, Oberwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/282,110

(22) PCT Filed: Mar. 6, 2007
(86) PCT No.: PCT/US07/05822

§ 371 Date: Sep. 8, 2008

Related U.S. Application Data
(60) Provisional application No. 60/780,951, filed on Mar. 8, 2006.

(51) Int. Cl. A61K 31/519; C07D 487/04

(52) U.S. Cl. 514/233.2; 544/281; 544/117; 514/252.16; 514/259.3

(57) ABSTRACT

The invention relates to methods of using the compounds of the invention, including pyrazolo[1,5a]pyrimidin-7-yl amine compounds and salts thereof, as well as pharmaceutical compositions comprising the same, in the treatment of Eph receptor-related (e.g., neurological) injuries and disorders. The invention also relates to modulating the activity of an Eph receptor in a cell, stimulating neural regeneration, and reversing neuronal degeneration, by administering a compound of the invention to a cell or subject in an effective amount.

### BACKGROUND OF THE INVENTION

Injury to the adult mammalian central nervous system (CNS) is often characterized by axonal impairment, including an inability of severed axons to regrow to their targets, resulting in permanent paralysis in subjects with said injuries. There currently exists no cure for patients who have suffered such CNS-related trauma as spinal cord injury (SCI), which is very often accompanied by debilitating clinical conditions like paraplegia or quadriplegia.

Axonal regeneration (e.g., post-injury) is prevented by a host of inhibitory influences in the adult CNS, among them inhibitory myelin proteins and the formation of a glial scar. Considerable progress has been made in identifying molecules associated with myelin inhibition (e.g., Nogo, myelin-associated glycoprotein (MAG), and oligodendrocyte-myelin glycoprotein (OMgp)), but relatively little is known about the glial scar which is formed as a response of glial cells to injury. (GrandPre, T., et al. (2000) Nature, 403(6768): 439; Fournier, A. E. et al. (2001) Nature 409(6818): 341; Wang, K. C., et al. (2002) Nature 417(6892): 941; and Wang, K. C., et al. (2002) Nature 420(6911): 74). Glial scarring is characterized by astrocytic gliosis, in which normally quiescent astrocytes proliferate and grow hypertrophic in response to injury, and otherwise form a physical and chemical barrier to axon regeneration. (Silver, J., et al. (2004) Nat Rev Neurosci 5(2): 146; and Morgenstern, D. A., et al. (2002) Prog Brain Res. 137: 313). Although a range of glial cells contribute to scar formation, the astrocytic response (i.e., astrocytic gliosis) is thought to be the primary mechanism for this occurrence.

The Eph receptor tyrosine kinase subfamily is thought to be the largest subfamily of transmembrane receptor tyrosine kinases, and with its ligands, the ephrins, is responsible for governing proper cell migration and positioning during neural development, presumably through modulating intercellular repulsion. (Pasquale, E. (1997) Curr. Opin. Cell Biol. 9:608-615)(Orioli and Klein (1997) Trends in Genetics 13:354-359). Eph receptors are closely related, and actively signal when bound to their ephrin ligands (their effects are mediated by cell-to-cell contacts), with which they are capable of both forward and bi-directional signaling. (Murai, K. K., et al. (2003) J Cell Sci. 116: 2823).

The Eph receptors are known regulators of neural development, with roles in the regulation of migrating cells or axons, the establishment of tissue patterns and topographic maps in distinct regions of the developing brain, and the regulation of synapse formation and plasticity. Eph receptors, including EphA4 and EphA7, are upregulated after spinal cord damage or deafferentation. (Miranda, et al. (1999) Exp Neurol 156:218; Willson, et al. (2002) Cell Transplantation 11:229); therefore, their inhibition is viewed as a potential therapeutic strategy for the treatment of neurological disorders.

A significant step toward curing or ameliorating complications resulting from spinal cord injury has been wanting, owing to the complex and multi-factorial nature of SCIs. In vivo studies have been performed to assess recovery following SCI by blocking either myelin inhibitors (GrandPre, T., et al. (2002) Nature 417(6888): 547; Kim, J. E., et al. (2003) Neuron 38(2): 187), chondroitin sulfate proteoglycans (Bradbury, E. J., et al. (2002) Nature 416 (6881): 636) or signaling molecules downstream of both of these (Fournier, A. E., et al. (2003) J. Neurosci. 23(4): 1416; and Sivasankaran, R., et al. (2004) Nat. Neurosci. 7(3): 261), with only marginal success. Experimental inhibition of Eph receptors, however, has revealed considerable axon regeneration following injury and suppressed astrocytic gliosis, leading to a dramatic reduction in glial scarring, and making these receptors an ideal therapeutic target for spinal cord injury and stroke, which also results in axonal damage and gliosis. Strategies and therapeutics designed to block Eph receptor function therefore herald a significant advance in the treatment of CNS-related disorders, and could presumably lead to vastly improved recovery following neural injury such as SCI, stroke and other neurodegenerative disorders.

### SUMMARY OF THE INVENTION

The invention relates to methods of using of the compounds of the invention for the treatment of Eph receptor-related (e.g., neurological) injuries and disorders, and methods of using pharmaceutical preparations comprising the compounds of the invention in the treatment of Eph receptor-related (e.g., neurological) injuries and disorders.

The invention also relates to methods of modulating the activity of an Eph receptor in a cell by contacting the cell with an effective amount of the compounds of the invention. In certain embodiments, Eph receptors can be modulated either in vitro or in vivo.

The invention also relates to methods of stimulating and promoting neural regeneration (such as axon regeneration following spinal cord injury), and reversing neuronal degeneration due to traumatic injury, hypoxic conditions, or infarct (e.g., as in stroke or nerve degeneration that is an underlying cause in multiple sclerosis and other neurodegenerative diseases). One way in which this can be achieved is through the administration to a mammal of a compound of the invention in an amount that is sufficient to stimulate and promote neural regeneration (such as axon regeneration) or reverse neuronal degeneration. The compounds of the invention can be delivered to both normal and injured cells. In some embodiments, the compounds of the invention inhibit the phosphorylation of an Eph receptor. In other embodiments, the compounds of the invention inhibit the binding of ephrin ligands to Eph receptors.

The invention also relates to methods for delivering a therapeutic agent to a cell, such as via a conjugate which comprises a therapeutic agent (e.g., a linking reagent) linked to compound of the invention.

As described herein and in PCT publication WO05/070431 (the contents of which are hereby incorporated by reference), the compounds of the invention, e.g., pyrazolo[1,5a]pyrimidin-7-yl amine derivatives, are, among other things, useful as protein kinase inhibitors and thus in the treatment of protein kinase-related disorders. By way of example, the compounds of the invention are useful as receptor tyrosine kinase inhibitors, such as Ephrin receptor kinase inhibitors, and can therefore be used to treat, e.g., neurological injuries and disorders.

### BRIEF DESCRIPTION OF THE FIGURE(S)

FIGS. 1A and 1B show inhibition of EphA4 auto-phosphorylation and ligand-dependent phosphorylation, respectively (from samples subjected to EphA4 immunoprecipitation followed by a phospho-tyrosine Western blot). FIG. 1A, lanes and 2 represent samples from cells treated with either control IgG-Fc or with the ligand ephrinB3-Fc. Lanes 2-6 represent samples from cells that have been pre-treated with Compound 1 (100 nM) and are then stimulated with IgG-Fc or ephrinB3-Fc in the combined presence of the inhibitor (Compound I)(lanes 3, 4) or in its absence (lanes 5, 6). FIG. 1B, lanes 1 and 2 show EphA4 phosphorylation in control (untreated) and ephrinB3-Fc stimulated cells following serum starvation. All other lanes represent samples from cells stimulated with ephrinB3-Fc in the presence of varying concentrations (as indicated) of tested Eph inhibitors.

FIG. 2A shows immunofluoroscence images demonstrating that Eph receptor inhibitors (e.g., Compound I (100 nM)) are able to overcome neurite outgrowth inhibition at nanomolar concentrations. FIG. 2B shows a graphical representation of experimentally-determined neurite outgrowth inhibition. The Y axis shows average neurite length in microns.

FIG. 3 shows a graphical representation of the experimental determination that Eph receptor inhibitors block astrocyte migration induced by cytokines (TGF-α, LIF, and IFN). The Y axis shows average, relative distance of cell migration compared to control (serum free=1). The white bar represents the addition of Compound 6. The black bar represents no compounds added.

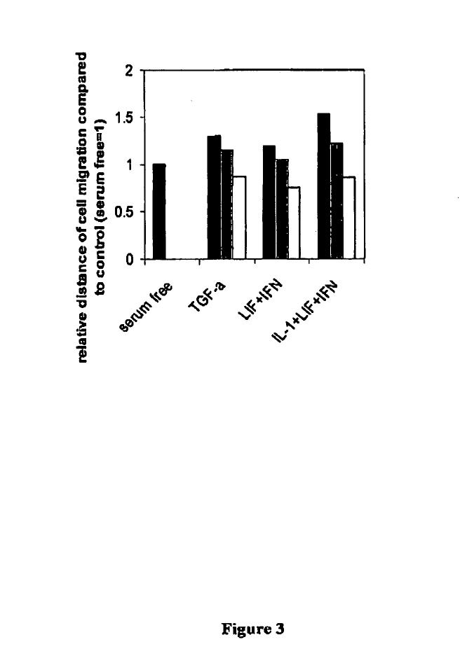

FIG. 4 demonstrates that Eph receptor inhibitors block EphA4 phosphorylation in vivo in mouse brain (brain homogenate lysates were subjected to EphA4 immunoprecipitation followed by a phospho-tyrosine Western blot). Animals were given an i.v dose of relevant compound and sacrificed 25 minutes or 1 hour after dosing (0.25 h or 1 h), the brains removed and subjected to EphA4 immunoprecipitation followed by a phospho-tyrosine Western blot. Four animals were used as controls and three-animals each were used per time point for each drug. From top to bottom, the compounds tested were Compound 1, Compound 7, and Compound 6.

FIGS. 5A and 5B shows methods of preparation of the compounds of formula (I). As described below, FIG. 5B describes the preparation of compounds of formula (I) beginning with synthesizing the pyrazolo[1,5-a]pyrimidin-7-ylamine core scaffold carrying a corresponding functional group (X) where residues A, R₂, or R₃, respectively, can be introduced by known reactions as indicated.

FIG. 6 shows a method of preparation of Compounds 9 and 10.

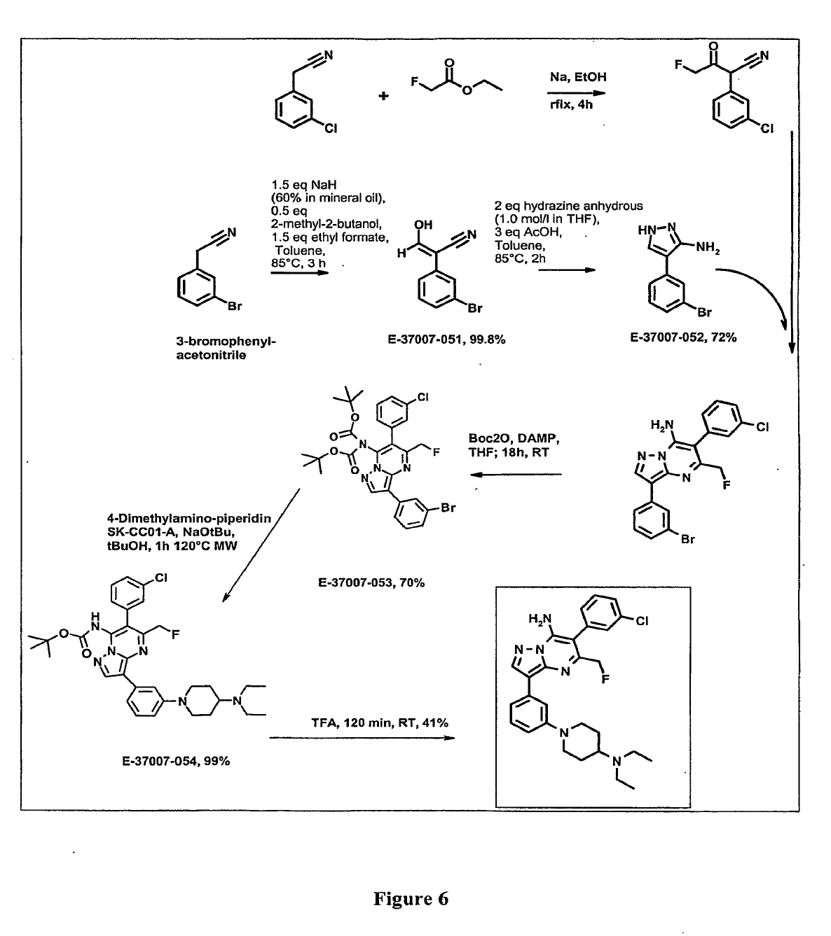

### DETAILED DESCRIPTION OF THE INVENTION

The present invention relates to compounds of the invention, including pyrazolo[1,5a]pyrimidin-7-yl amine compounds, of the formula (I):

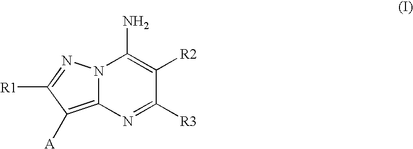

wherein:

R₂ is H, substituted or unsubstituted aryl, substituted or unsubstituted heteroaryl, substituted or unsubstituted aliphatic residue, a functional group, or a substituted or unsubstituted aryl, substituted or unsubstituted heteroaryl or substituted or unsubstituted aliphatic residue which is connected by one connecting group or atom to the pyrazolo[1,5a]pyrimidinyl ring;

R₃ is H, substituted or unsubstituted aryl, substituted or unsubstituted heteroaryl, substituted or unsubstituted aliphatic residue, a functional group, or an aliphatic residue which may be connected by a connecting group or atom to the pyrazolo[1,5a]pyrimidinyl ring,

at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;

A is H, halogen (such as bromo), an aliphatic moiety, a functional group, substituted or unsubstituted aryl or heteroaryl; and

R₁ is H, halogen or lower alkyl,

or pharmaceutically acceptable salts thereof, in the treatment of Eph receptor-related (e.g., neurological) injuries and disorders or for the manufacture of pharmaceutical compositions for use in the treatment of said injuries and disorders, methods of use of compounds of formula (I) in the treatment of said injuries and disorders, pharmaceutical preparations comprising compounds of formula (I) for the treatment of said injuries and disorders, compounds of formula (I) for use in the treatment of said diseases.

A preferred embodiment is the use of a compound according to the above, wherein:

R₂ is H; lower alkyl; cycloalkyl; benzyl; benzo thienyl, indyl substituted by lower alkyl, pyridyl or thiazolyl optionally substituted by lower alkyl; unsubstituted phenyl or phenyl substituted by one or two substituents chosen from the group consisting of; halo, hydroxy, alkoxy, benzyloxy, cycloalkyl, amino, acetyl amino, lower alkyl sulfonamide and benzene sulfonamide substituted by one or two halo;

R₃ is H; lower alkyl optionally substituted by halo; phenyl, pyridyl, or oxazolyl;

A is: (a) H; halo; benzothienyl; pyridyl; methyl piperazinyl phenoxyl; indolyl substituted with lower alkyl;

(b) phenyl which is unsubstituted or substituted with one or more of the substituents chosen from the group consisting of; mono-, di- or tri-lower alkoxy, di-lower alkylaminyl, morpholinyl which is optionally di-substituted by alkyl, piperazinyl which is substituted with one or more of the substituents chosen from the group consisting of lower alkyl, lower alkoxy, lower alkyl piperazinyl, pyrrolidinyl, dialkyl aminyl and lower alkanol; and

R₁ is H, or pharmaceutically acceptable salts thereof for treating Eph receptor-related (e.g., neurological) injuries and disorders.

As described herein, the compounds of the invention, e.g., compounds of formula (I), e.g., pyrazolo[1,5a]pyrimidin-7-yl amine derivatives, are, among other things, useful in the treatment of Eph receptor-related (e.g., neurological) injuries and disorders.

Pyrazolo[1,5a]pyrimidin-7-yl amine derivatives have demonstrated surprisingly pharmaceutically advantageous properties, inter alia allowing for the inhibition of specific types or classes or groups of kinases, including Eph receptor kinases. In addition to this established activity, the pyrazolo[1,5a]pyrimidin-7-yl amine derivatives have the advantage that their backbone in addition allows for substitution patterns that offer a broad possibility to achieve a fine tuning for specific interaction with the binding site of the targeted kinase or kinases, thus opening a new perspective and providing kinase inhibitors of various degrees of specificity. In view of these activities, the compounds of the invention can be used for the treatment of diseases related to especially aberrant or excessive activity of such types of kinases, e.g., Eph receptor-related (e.g., neurological) injuries and disorders.

Most preferably, the disease to be treated is neurological injury or disorder, as described in greater detail herein.

In a further embodiment, the invention relates to a compound of formula (I):

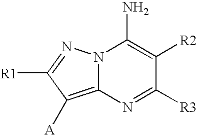

wherein:

R₂ is H; substituted or unsubstituted aryl; substituted or unsubstituted heteroaryl; an aliphatic residue; a functional group; or a substituted or unsubstituted aryl, substituted or unsubstituted heteroaryl or aliphatic residue which is connected by one connecting group or atom to the pyrazolo[1,5a]pyrimidinyl ring;

R₃ can be H, substituted or unsubstituted aryl, heteroaryl, an aliphatic residue, a functional group, or an aliphatic residue which may be connected by a connecting group or atom to the pyrazolo[1,5a]pyrimidinyl ring,

at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring, and provided that both R₂ and A cannot both be unsubstituted phenyl;

A is H, halogen (such as bromo), an aliphatic moiety, a functional group, substituted or unsubstituted aryl or substituted or unsubstituted heteroaryl; and

R₁ is H, halogen or lower alkyl,

or a pharmaceutically acceptable salt thereof.

A preferred embodiment is a compound according to the above, wherein;

R₂ is H; lower alkyl; cycloalkyl; benzyl; benzo thienyl, indyl substituted by lower alkyl, pyridyl or thiazolyl optionally substituted by lower alkyl; unsubstituted phenyl or phenyl substituted by one or two substituents chosen from the group consisting of; halo, hydroxy, alkoxy, benzyloxy, cycloalkyl, amino, acetyl amino, lower alkyl sulfonamide and benzene sulfonamide substituted by one or two halo;

R₃ is H; lower alkyl optionally substituted by halo; phenyl, pyridyl, or oxazolyl;

A is

H; halo; benzothienyl; pyridyl; methyl piperazinyl phenoxyl; indolyl substituted with lower alkyl;

(b) phenyl which is unsubstituted or substituted with one or more of the substituents chosen from the group consisting of; mono-, di- or tri-lower alkoxy, di-lower alkylaminyl, morpholinyl which is optionally di-substituted by alkyl,

piperazinyl which is substituted with one or more of the substituents chosen from the group consisting of lower alkyl, lower alkoxy, lower alkyl piperazinyl, pyrrolidinyl, dialkyl-aminyl and lower alkanol; and

R₁ is H; and provided that both R2 and A cannot both be unsubstituted phenyl.

Most preferably, the compound is selected from the group consisting of:
6-(3-Chloro-phenyl)-3-[3-(4-diethylamino-piperidin-1-yl)-phenyl]-5-methyl-pyrazolo[1,5-a]pyrimidin-7-ylamine (also referred to herein as “Compound 1”);
6-(3-Chloro-phenyl)-3-(3,4-dimethoxy-phenyl)-5-methyl-pyrazolo[1,5-a]pyrimidin-7-ylamine (also referred to herein as “Compound 3”);
2-(4-{3-[7-Amino-6-(3-chloro-4-fluoro-phenyl)-5-methyl-pyrazolo[1,5-a]pyrimidin-3-yl]-phenyl}-piperazin-1-yl)-ethanol (also referred to herein as “Compound 4”);
6-(3-Chloro-phenyl)-5-methyl-3-{3-[4-(1-methyl-piperidin-4-yl)-piperazin-1-yl]-phenyl}-pyrazolo[1,5-a]pyrimidin-7-ylamine (also referred to herein as “Compound 5”);
6-(3-Chloro-7-phenyl)-3-(3,4-dimethoxy-phenyl)-5-piperazin-1-ylmethyl-pyrazolo[1,5-a]pyrimidin-7-ylamine (also referred to herein as “Compound 6”);
6-(3-Chloro-phenyl)-3-[4-(2-dimethylamino-ethoxy)-phenyl]-5-methyl-1-pyrazolo[1,5-a]pyrimidin-7-ylamine (also referred to herein as “Compound 7”);
N-{3-[1-(4-Methoxy-phenyl)-1H-pyrazolo[3,4-d]pyrimidin-4-ylamino]-4-methyl-phenyl}-3-trifluoromethyl-benzamide (also referred to herein as “Compound 8”);
3-(3-Bromo-phenyl)-6-(3-chloro-phenyl)-5-fluoromethyl-pyrazolo[1,5-a]pyrimidin-7-ylamine (also referred to herein as “Compound 9”);
6-(3-Chloro-phenyl)-3-[3-(4-diethylamino-piperidin-1-yl)-phenyl]-5-fluoromethyl-pyrazolo[1,5-a]pyrimidin-7-ylamine (also referred to herein as “Compound 10”);
3-{7-Amino-3-[4-(4-methyl-piperazin-1-yl)-phenyl]-pyrazolo[1,5-a]pyrimidin-6-yl}-phenol;
6-(3-benzyloxy-phenyl)-3-[4-(4-methyl-piperazin-1-yl)-phenyl]-pyrazolo[1,5-a]pyrimidin-7-yl}-phenol;
6-(3-Methoxy-phenyl)-3-[4-(4-methyl-piperazin-1-yl)-phenyl]-pyrazolo[1,5-a]pyrimidin-7-ylamine;
6-(3,5-Dimethoxy-phenyl)-3-[4-(4-methyl-piperazin-1-yl)-phenyl]-pyrazolo[1,5-a]pyrimidin-7-ylamine;
6-(3-Benzyloxy-phenyl)-3-[4-(4-methyl-piperazin-1-yl)-phenyl]-pyrazolo[1,5-a]pyrimidin-7-ylamine;
6-(4-Chloro-phenyl)-3-[4-(4-methyl-piperazin-1-yl)-phenyl]-pyrazolo[1,5-a]pyrimidin-7-ylamine;
6-(3-Chloro-phenyl)-3-[4-(4-methyl-piperazin-1-yl)-phenyl]-pyrazolo[1,5-a]pyrimidin-7-ylamine;
3-[4-(4-Methyl-piperazin-1-yl)-phenyl]-6-phenyl-pyrazolo[1,5-a]pyrimidin-7-ylamine,
5-Methyl-3-[4-(4-methyl-piperazin-1-yl)-phenyl]-6-phenyl-pyrazolo[1,5-a]pyrimidin-7-ylamine;
6-Methyl-3-[4-(4-methyl-piperazin-1-yl)-phenyl]-5-phenyl-pyrazolo[1,5-a]pyrimidin-7-ylamine;
N-{4-[7-Amino-3-(4-dimethylamino-phenyl)-pyrazolo[1,5-a]pyrimidin-6-yl]-phenyl}-2,3-dichloro-benzenesulfonamide;
4-Chloro-benzenesulfonic acid 4-[7-amino-3-(4-dimethylamino-phenyl)-pyrazolo[1,5-a]pyrimidin-6-yl]-phenyl ester;
6-(4-Methoxy-phenyl)-5-methyl-3-phenyl-pyrazolo[1,5-a]pyrimidin-7-ylamine;
3-(4-Methoxy-phenyl)-5-methyl-6-phenyl-pyrazolo[1,5-a]pyrimidin-7-ylamine;
6-(4-Bromo-phenyl)-3-(4-methoxy-phenyl)-5-methyl-pyrazolo[1,5-a]pyrimidin-7-ylamine;
6-(4-Bromo-phenyl)-5-methyl-3-phenyl-pyrazolo[1,5-a]pyrimidin-7-ylamine;
6-(2,6-Dichloro-phenyl)-3-phenyl-pyrazolo[1,5-a]pyrimidin-7-ylamine;
3-(3-Methoxy-phenyl)-6-phenyl-pyrazolo[1,5-a]pyrimidin-7-ylamine;
3-Bromo-5-phenyl-pyrazolo[1,5-a]pyrimidin-7-ylamine;
6-Benzo[b]thiophen-3-yl-3-[4-(4-methyl-piperazin-1-yl)-phenyl]-pyrazolo[1,5-a]pyrimidin-7-ylamine;
3-(4-Bromo-phenyl)-5-phenyl-pyrazolo[1,5-a]pyrimidin-7-ylamine;
3-[4-(4-Methyl-piperazin-1-yl)-phenyl]-6-thiophen-3-yl-pyrazolo[1,5-a]pyrimidin-7-ylamine;
3-Benzo[b]thiophen-3-yl-6-(3-methoxy-phenyl)-pyrazolo[1,5-a]pyrimidin-7-ylamine;
6-Benzo-3-[4-(4-methyl-piperazin-1-yl)-phenyl]-pyrazolo[1,5-a]pyrimidin-7-ylamine;
6-(3-Methoxy-phenyl)-3-[3-(4-methyl-piperazin-1-yl)-phenyl]-pyrazolo[1,5-a]pyrimidin-7-ylamine;
6-(1-Methyl-1H-indol-3-yl)-3-[4-(4-methyl-piperazin-1-yl)-phenyl]-pyrazolo[1,5-a]pyrimidin-7-ylamine;
6-(4-Methoxy-phenyl)-3-[4-(4-methyl-piperazin-1-yl)-phenyl]-pyrazolo[1,5-a]pyrimidin-7-ylamine;
6-(2-Methoxy-phenyl)-3-[4-(4-methyl-piperazin-1-yl)-phenyl]-pyrazolo[1,5-a]pyrimidin-7-ylamine;
6-(3-Methoxy-phenyl)-3-pyridin-3-yl-pyrazolo[1,5-a]pyrimidin-7-ylamine;
3-{7-Amino-3-[3-(4-methyl-piperazin-1-yl)-phenyl]-pyrazolo[1,5-a]pyrimidin-6-yl}-phenol;
6-(3-Benzyloxy-phenyl)-3-[2-methoxy-5-(4-methyl-piperazin-1-yl)-phenyl]-pyrazolo[1,5-a]pyrimidin-7-ylamine;
3-{7-Amino-3-[2-methoxy-5-(4-methyl-piperazin-1-yl)-phenyl]-pyrazolo[1,5-a]pyrimidin-6-yl}-phenol;
6-(2-Benzyloxy-phenyl)-3-[4-(4-methyl-piperazin-1-yl)-phenyl]-pyrazolo[1,5-a]pyrimidin-7-ylamine;
2-{7-Amino-3-[4-(4-methyl-piperazin-1-yl)-phenyl]-pyrazolo[1,5-a]pyrimidin-6-yl}-phenol;
6-(4-Benzyloxy-phenyl)-3-[4-(4-methyl-piperazin-1-yl)-phenyl]-pyrazolo[1,5-a]pyrimidin-7-ylamine;
4-{7-Amino-3-[4-(4-methyl-piperazin-1-yl)-phenyl]-pyrazolo[1,5-a]pyrimidin-6-yl}-phenol;
6-(2-Benzyloxy-phenyl)-3-[3-(4-methyl-piperazin-1-yl)-phenyl]-pyrazolo[1,5-a]pyrimidin-7-ylamine;
2-{7-Amino-3-[3-(4-methyl-piperazin-1-yl)-phenyl]-pyrazolo[1,5-a]pyrimidin-6-yl}-phenol;
6-(4-Benzyloxy-phenyl)-3-[3-(4-methyl-piperazin-1-yl)-phenyl]-pyrazolo[1,5-a]pyrimidin-7-ylamine;
4-{7-Amino-3-[3-(4-methyl-piperazin-1-yl)-phenyl]-pyrazolo[1,5-a]pyrimidin-6-yl}-phenol;
6-(2-Benzyloxy-phenyl)-3-[2-methoxy-5-(4-methyl-piperazin-1-yl)-phenyl]-pyrazolo[1,5-a]pyrimidin-7-ylamine;
2-{7-Amino-3-[2-methoxy-5-(4-methyl-piperazin-1-yl)-phenyl]-pyrazolo[1,5-a]pyrimidin-6-yl}-phenol;
6-(4-Benzyloxy-phenyl)-3-[2-methoxy-5-(4-methyl-piperazin-1-yl)-phenyl)-pyrazololo[1,5-a]pyrimidin-7-ylamine;
4-{7-Amino-3-[2-methoxy-5-(4-methyl-piperazin-1-yl)-phenyl]-pyrazolo[1,5-a]pyrimidin-6-yl}-phenol;
6-(3-Benzyloxy-phenyl)-3-[1-methyl-1H-indol-3-yl)-pyrazolo[1,5-a]pyrimidin-7-ylamine;
3-[7-Amino-3-(1-methyl-1H-indol-3-yl)-pyrazolo[1,5-a]pyrimidin-6-yl]-phenol;
3-[7-Amino-3-pyridin-3-yl-pyrazolo[1,5-a]pyrimidin-6-yl]-phenol;
6-(3-Benzyloxy-phenyl)-3-(2-methoxy-phenyl)-pyrazolo[1,5-a]pyrimidin-7-ylamine;
3-[1-Amino-3-(2-methoxy-phenyl)-pyrazolo[1,5-a]pyrimidin-6-yl]-phenol;
3-[3-(4-Methyl-piperazin-1-yl)-phenyl]-6-thiophen-3-yl-pyrazolo[1,5-a]pyrimidin-7-ylamine;
3-(2-Methoxy-5-(4-methyl-piperazin-1-yl)-phenyl]-6-thiophen-3-yl-pyrazolo[1,5-a]pyrimidin-7-ylamine;
3-[4-(4-Methyl-piperazin-1-yl)-phenyl]-6-pyridin-4-yl-pyrazolo[1,5-a]pyrimidin-7-ylamine;
6-(3-Amino-phenyl)-3-[3-(4-methyl-piperazin-1-yl)-phenyl]-pyrazolo[1,5-a]pyrimidin-7-ylamine;
6-(3-Amino-phenyl)-3-[4-(4-methyl-piperazin-1-yl)-phenyl]-pyrazolo[1,5-a]pyrimidin-7-ylamine;
6-(2-Amino-phenyl)-3-[4-(4-methyl-piperazin-1-yl)-phenyl]-pyrazolo[1,5-a]pyrimidin-7-ylamine;
3-[4-(4-Methyl-piperazin-1-yl)-phenyl]-6-(4-methyl-thiazol-2-yl)-pyrazolo[1,5-a]pyrimidin-7-ylamine;
6-Benzo[b]thiophen-3-yl-3-[2-methoxy-5-(4-methyl-piperazin-1-yl)-phenyl]-pyrazolo[1,5-a]pyrimidin-7-ylamine;
6-Benzo[b]thiophen-3-yl-3-[4-methoxy-phenyl)-pyrazolo[1,5-a]pyrimidin-7-ylamine;
3-(3-Methoxy-phenyl)-6-thiophen-3-yl-pyrazolo[1,5-a]pyrimidin-7-ylamine;
6-(3-Benzyloxy-phenyl)-3-(3-methoxy-phenyl)-pyrazolo[1,5-a]pyrimidin-7-ylamine;
3-[7-Amino-3-(3-methoxy-phenyl)-pyrazolo[1,5-a]pyrimidin-6-yl]-phenol;
(4-{7-Amino-3-[4-(4-methyl-piperazin-1-yl)-phenyl]-pyrazolo[1,5-a]pyrimidin-6-yl}-phenyl)-carbamic acid ethyl ester.
6-(3-Chloro-phenyl)-5-methyl-3-[3-(4-methyl-piperazin-1-yl)-phenyl]-pyrazolo[1,5-a]pyrimidin-7-ylamine;
6-(3-Chloro-phenyl)-5-methyl-3-[4-(4-methyl-piperazin-1-yl)-phenyl]-pyrazolo[1,5-a]pyrimidin-7-ylamine;
6-(3-Chloro-phenyl)-3-[2-methoxy-5-(4-methyl-piperazin-1-yl)-phenyl]-5-methyl-pyrazolo[1,5-a]pyrimidin-7-ylamine;
6-(3-Chloro-phenyl)-3-[2-methoxy-4-(4-methyl-piperazin-1-yl)-phenyl]-5-methyl-pyrazolo[1,5-a]pyrimidin-7-ylamine;
3-{7-Amino-3-[2-methoxy-4-(4-methyl-piperazin-1-yl)-phenyl]-pyrazolo[1,5-a]pyrimidin-7-phenol;
6-(2-Chloro-phenyl)-3-[4-(4-methyl-piperazin-1-yl)-phenyl]-pyrazolo[1,5-a]pyrimidin-7-ylamine;
6-(2-Chloro-phenyl)-3-[3-(4-methyl-piperazin-1-yl)-phenyl]-pyrazolo[1,5-a]pyrimidin-7-ylamine;
6-(4-Fluoro-phenyl)-5-methyl-3-[4-(4-methyl-piperazin-1-yl)-phenyl]-pyrazolo[1,5-a]pyrimidin-7-ylamine;
6-(4-Fluoro-phenyl)-5-methyl-3-[3-(4-methyl-piperazin-1-yl)-phenyl]-pyrazolo[1,5-a]pyrimidin-7-ylamine;
6-(3-Chloro-4-fluoro-phenyl)-5-methyl-3-[3-(4-methyl-piperazin-1-yl)-phenyl]pyrazolo[1,5-a]pyrimidin-7-ylamine;
6-(3-Chloro-4-fluoro-phenyl)-5-methyl-3-[4-(4-methyl-piperazin-1-yl)-phenyl]-pyrazolo[1,5-a]pyrimidin-1-ylamine;
6-(3-Bromo-phenyl)-5-methyl-3-[3-(4-methyl-piperazin-1-yl)-phenyl]-pyrazolo[1,5-a]pyrimidin-7-ylamine;
6-(3-Bromo-benzyl)-3-[3-(4-methyl-piperazin-1-yl)-phenyl]-pyrazolo[1,5-a]pyrimidin-7-ylamine;
6-(3-Bromo-phenyl)-3-[3-(4-methyl-piperazin-1-yl)-phenyl]-pyrazolo[1,5-a]pyrimidin-7-ylamine;
6-(3-Chloro-phenyl)-5-methyl-3-(3-morpholin-4-yl-phenyl)-pyrazolo[1,5-a]pyrimidin-7-ylamine;
6-(3-Chloro-phenyl)-3-(4-methoxy-phenyl)-5-methyl-pyrazolo[1,5-a]pyrimidin-7-ylamine;
6-(3-Chloro-phenyl)-3-[3-((2R,6S)-2,6-dimethyl-morpholin-4-yl)-phenyl]-5-methyl-pyrazolo[1,5-a]pyrimidin-7-ylamine;
2-(4-{3-[7-Amino-6-(3-chloro-phenyl)-5-methyl-pyrazolo[1,5-a]pyrimidin-3-yl]-phenyl}-piperazin-1-yl)-ethanol;
6-Benzyl-3-[3-(4-methyl-piperazin-1-yl)-phenyl]-pyrazolo[1,5-a]pyrimidin-7-ylamine;
6-(3-Chloro-phenyl)-3-(3,4-dimethoxy-phenyl)-5-fluoromethyl-pyrazolo[1,5-a]pyrimidin-7-ylamine;
6-(3-Chloro-4-fluoro-phenyl)-3-(3,4-dimethoxy-phenyl)-5-methyl-pyrazolo[1,5-a]pyrimidin-7-ylamine;
6-(3-Chloro-4-fluoro-phenyl)-3-(4-methoxy-phenyl)-5-methyl-pyrazolo[1,5-a]pyrimidin-7-ylamine;
6-(4-Fluoro-phenyl)-3-(4-methoxy-phenyl)-5-methyl-pyrazolo[1,5-a]pyrimidin-7-ylamine;
2-(4-{3-[7-Amino-6-(4-fluoro-phenyl)-5-methyl-pyrazolo[1,5-a]pyrimidin-3-yl]-phenyl}-piperazin-1-yl)-ethanol;
6-(3,4-Difluoro-phenyl)-5-methyl-3-[3-(4-methyl-piperazin-1-yl)-phenyl]-pyrazolo[1,5-a]pyrimidin-7-ylamine;
6-(3,4-Difluoro-phenyl)-3-(3,4-dimethoxy-phenyl)-5-methyl-pyrazolo[1,5-a]pyrimidin-7-ylamine;
2-(4-{3-[7-Amino-6-(3-chloro-4-fluoro-phenyl)-5-methyl-pyrazolo[1,5-a]pyrimidin-3-yl]-phenyl}-piperazin-1-yl)-ethanol;
2-(4-{3-[7-Amino-6-(3,4-difluoro-phenyl)-5-methyl-pyrazolo[1,5-a]pyrimidin-3-yl]-phenyl}-piperazin-1-yl)-ethanol;
6-(3-Chloro-phenyl)-5-methyl-3-[3-(4-pyrrolidin-1-yl-piperidin-1-yl)-phenyl]-pyrazolo[1,5-a]pyrimidin-7-ylamine;
6-(4-Fluoro-phenyl)-5-methyl-3-[3-(4-pyrrolidin-1-yl-piperidin-1-yl)-phenyl]-pyrazolo[1,5-a]pyrimidin-7-ylamine;
3-[3-(4-Diethylamino-piperidin-1-yl)-phenyl]-6-(4-fluoro-phenyl)-5-methyl-pyrazolo[1,5-a]pyrimidin-7-ylamine;
6-(4-Fluoro-phenyl)-5-methyl-3-[3-(4-methyl-4-oxy-piperazin-1-yl)-phenyl]-pyrazolo[1,5-a]pyrimidin-7-ylamine;
6-(4-Fluoro-phenyl)-5-methyl-3-[3-(4-methyl-1,4-dioxy-piperazin-1-yl)-phenyl]-pyrazolo[1,5-a]pyrimidin-7-ylamine;
6-(3-Chloro-phenyl)-3-[3-(4-dimethylamino-piperidin-1-yl)-phenyl]-5-methyl-pyrazolo[1,5-a]pyrimidin-7-ylamine;
6-(3,4-Difluoro-phenyl)-3-[3-(4-dimethylamino-piperidin-1-yl)-phenyl]-5-methyl-pyrazolo[1,5-a]pyrimidin-7-ylamine;
6-(3-Chloro-phenyl)-5-methyl-3-(3,4,5-trimethoxy-phenyl)-pyrazolo[1,5-a]pyrimidin-7-ylamine; 6-(3,4-Difluoro-phenyl)-5-methyl-3-(3,4,5-trimethoxy-phenyl)-pyrazolo[1,5-a]pyrimidin-7-ylamine;
6-(3-Chloro-phenyl)-3-(3-methoxy-phenyl)-5-methyl-pyrazolo[1,5-a]pyrimidin-7-ylamine;
6-[7-Amino-3-(3,4-dimethoxy-phenyl)-pyrazolo[1,5-a]pyrimidin-6-yl]-pyridin-2-ol;
6-Benzyl-3-(3,4-dimethoxy-phenyl)-pyrazolo[1,5-a]pyrimidin-7-ylamine;
3-(3,4-Dimethoxy-phenyl)-6-(3-fluoro-benzyl)-pyrazolo[1,5-a]pyrimidin-7-ylamine;
6-(3-Chloro-phenyl)-3-(2-methoxy-5-piperazin-1-yl-phenyl)-5-methyl-pyrazolo[1,5-a]pyrimidin-7-ylamine;
6-(3-Chloro-phenyl)-3-(2-methoxy-5-morpholin-4-yl-phenyl)-5-methyl-pyrazolo[1,5-a]pyrimidin-7-ylamine;
3-(2-Methoxy-5-morpholin-4-yl-phenyl)-5-methyl-6-(3-morpholin-4-yl-phenyl)-pyrazolo[1,5-a]pyrimidin-7-ylamine;
N-{3-[7-Amino-6-(3-chloro-phenyl)-5-methyl-pyrazolo[1,5-a]pyrimidin-3-yl]-4-methoxy-phenyl}-N,N′,N′-trimethyl-ethane-1,2-diamine;
N-{3-[7-Amino-6-(3-chloro-phenyl)-5-methyl-pyrazolo[1,5-a]pyrimidin-3-yl]-4-methoxy-phenyl}-N,N′,N′-trimethyl-propane-1,3-diamine;
6-(3-Chloro-phenyl)-3-[5-(4-diethylamino-piperidin-1-yl)-2-methoxy-phenyl]-5-methyl-pyrazolo[1,5-a]pyrimidin-7-ylamine;
6-(3-Chloro-phenyl)-3-{2-methoxy-5-[4-(1-methyl-piperidin-4-yl)-piperazin-1-yl]-phenyl}-5-methyl-pyrazolo[1,5-a]pyrimidin-7-ylamine;
6-(3-Chloro-phenyl)-3-[2-ethoxy-5-(4-pyrrolidin-1-yl-piperidin-1-yl)-phenyl]-5-methyl-pyrazolo[1,5-a]pyrimidin-7-ylamine;
N-{3-[7-Amino-6-(3-chloro-phenyl)-5-methyl-pyrazolo[1,5-a]pyrimidin-3-yl]-4-methoxy-phenyl}-N′,N′-dimethyl-ethane-1,2-diamine;
N-{3-[7-Amino-6-(3-chloro-phenyl)-5-methyl-pyrazolo[1,5-a]pyrimidin-3-yl]-4-methoxy-phenyl}-N′,N′-dimethyl-propane-1,3-diamine;
3-[5-(4-Amino-piperidin-1-yl)-2-methoxy-phenyl]-6-(3-chloro-phenyl)-5-methyl-pyrazolo[1,5-a]pyrimidin-7-ylamine;
6-(3-Chloro-phenyl)-3-{2-methoxy-5-[4-(4-methyl-piperazin-1-yl)-piperidin-1-yl]-phenyl}-5-methyl-pyrazolo[1,5-a]pyrimidin-7-ylamine;
2-(4-{3-[7-Amino-6-(3-chloro-phenyl)-5-methyl-pyrazolo[1,5-a]pyrimidin-3-yl]-4-methoxy-phenyl}-piperazin-1-yl)-ethanol;
6-(3-Chloro-phenyl)-3-[5-(4-dimethylamino-piperidin-1-yl)-2-methoxy-phenyl]-5-methyl-pyrazolo[1,5-a]pyrimidin-7-ylamine;
6-(3-Chloro-phenyl)-3-[2-methoxy-5-(1-methyl-piperidin-4-ylamino)-phenyl]-5-methyl-pyrazolo[1,5-a]pyrimidin-7-ylamine;
3-(5-[1,4′]Bipiperidinyl-1′-yl-2-methoxy-phenyl)-6-(3-chloro-phenyl)-5-methyl-pyrazolo[1,5-a]pyrimidin-7-ylamine;
6-(3-Chloro-phenyl)-3-[2-fluoro-5-(4-methyl-piperazin-1-yl)-phenyl]-5-methyl-pyrazolo[1,5-a]pyrimidin-7-ylamine;
6-(3-Chloro-phenyl)-3-[5-(4-dimethylamino-piperidin-1-yl)-2-fluoro-phenyl]-5-methyl-pyrazolo[1,5-a]pyrimidin-7-ylamine;
6-(3-Chloro-phenyl)-3-[5-(4-diethylamino-piperidin-1-yl)-2-fluoro-phenyl]-5-methyl-pyrazolo[1,5-a]pyrimidin-7-ylamine;
6-(3-Chloro-phenyl)-3-(2-fluoro-5-morpholin-4-yl-phenyl)-5-methyl-pyrazolo[1,5-a]pyrimidin-7-ylamine;
6-(3-Chloro-phenyl)-3-[5-((2R,6S)-2,6-dimethyl-morpholin-4-yl)-2-fluoro-phenyl]-5-methyl-pyrazolo[1,5-a]pyrimidin-7-ylamine;
6-(3-Chloro-phenyl)-3-[2-fluoro-5-(4-pyrrolidin-1-yl-piperidin-1-yl)-phenyl]-5-methyl-pyrazolo[1,5-a]pyrimidin-7-ylamine;
6-(3-Chloro-phenyl)-3-[2-fluoro-5-(1-methyl-piperidin-4-ylamino)-phenyl]-5-methyl-pyrazolo[1,5-a]pyrimidin-7-ylamine;
6-(3-Chloro-phenyl)-3-{2-fluoro-5-[4-(4-methyl-piperazin-1-yl)-piperidin-1-yl]-phenyl}-5-methyl-pyrazolo[1,5-a]pyrimidin-7-ylamine;
6-(3-Chloro-phenyl)-3-{2-fluoro-5-[4-(1-methyl-piperidin-4-yl)-piperazin-1-yl]-phenyl}-5-methyl-pyrazolo[1,5-a]pyrimidin-7-ylamine;
2-(4-{3-[7-Amino-6-(3-chloro-phenyl)-5-methyl-pyrazolo[1,5-a]pyrimidin-3-yl]-4-fluoro-phenyl}-piperazin-1-yl)-ethanol;
3-[5-(4-Amino-piperidin-1-yl)-2-fluoro-phenyl]-6-(3-chloro-phenyl)-5-methyl-pyrazolo[1,5-a]pyrimidin-7-ylamine;
6-(3-Chloro-phenyl)-3-[2-fluoro-5-(piperidin-4-ylamino)-phenyl]-5-methyl-pyrazolo[1,5-a]pyrimidin-7-ylamine;
6-(3-Chloro-phenyl)-3-{2-fluoro-5-[methyl-(1-methyl-piperidin-4-yl)-amino]-phenyl}-5-methyl-pyrazolo[1,5-a]pyrimidin-7-ylamine;
N-{3-[7-Amino-6-(3-chloro-phenyl)-5-methyl-pyrazolo[1,5-a]pyrimidin-3-yl]-4-fluoro-phenyl}-N,N′,N′-triethyl-ethane-1,2-diamine;
N-{3-[7-Amino-6-(3-chloro-phenyl)-5-methyl-pyrazolo[1,5-a]pyrimidin-3-yl]-4-fluoro-phenyl}-N,N′,N′-trimethyl-propane-1,3-diamine;
N-{3-[7-Amino-6-(3-chloro-phenyl)-5-methyl-pyrazolo[1,5-a]pyrimidin-3-yl]-4-fluoro-phenyl}-N′,N′-dimethyl-ethane-1,2-diamine;
N-{3-[7-Amino-6-(3-chloro-phenyl)-5-methyl-pyrazolo[1,5-a]pyrimidin-3-yl]-4-fluoro-phenyl}-N′,N′-dimethyl-propane-1,3-diamine;
6-(3-Chloro-phenyl)-3-[5-(4-ethyl-piperazin-1-yl)-2-fluoro-phenyl]-5-methyl-pyrazolo[1,5-a]pyrimidin-7-ylamine;
6-(3-Chloro-phenyl)-3-[2-fluoro-5-(4-isopropyl-piperazin-1-yl)-phenyl]-5-methyl-pyrazolo[1,5-a]pyrimidin-7-ylamine;
N-{3-[7-Amino-6-(3-chloro-phenyl)-5-methyl-pyrazolo[1,5-a]pyrimidin-3-yl]-4-fluoro-phenyl}-N-ethyl-N′,N′-dimethyl-ethane-1,2-diamine;
N-{3-[7-Amino-6-(3-chloro-phenyl)-5-methyl-pyrazolo[1,5-a]pyrimidin-3-yl]-4-fluoro-phenyl}-N′-methyl-propane-1,3-diamine;
6-(3-Chloro-phenyl)-3-[2-fluoro-4-(4-methyl-piperazin-1-yl)-phenyl]-5-methyl-pyrazolo[1,5-a]pyrimidin-7-ylamine;
6-(3-Chloro-phenyl)-3-[4-(4-diethylamino-piperidin-1-yl)-2-fluoro-phenyl]-5-methyl-pyrazolo[1,5-a]pyrimidin-7-ylamine;
6-(3-Chloro-phenyl)-3-(2-fluoro-4-morpholin-4-yl-phenyl)-5-methyl-pyrazolo[1,5-a]pyrimidin-7-ylamine;
N-{4-[7-Amino-6-(3-chloro-phenyl)-5-methyl-pyrazolo[1,5-a]pyrimidin-3-yl]-3-fluoro-phenyl}-N,N′,N′-trimethyl-ethane-1,2-diamine;
6-(3-Chloro-phenyl)-3-[4-(4-dimethylamino-piperidin-1-yl)-2-fluoro-phenyl]-5-methyl-pyrazolo[1,5-a]pyrimidin-7-ylamine;
6-(3-Chloro-phenyl)-3-{2-fluoro-4-[4-(4-methyl-piperazin-1-yl)-piperidin-1-yl]-phenyl}-5-methyl-pyrazolo[1,5-a]pyrimidin-7-ylamine;
6-(3-Chloro-phenyl)-3-[4-((2R,6S)-2,6-dimethyl-morpholin-4-yl)-2-fluoro-phenyl]-5-methyl-pyrazolo[1,5-a]pyrimidin-7-ylamine;
N-{4-[7-Amino-6-(3-chloro-phenyl)-5-methyl-pyrazolo[1,5-a]pyrimidin-3-yl]-3-fluoro-phenyl}-N,N′,N′-trimethyl-propane-1,3-diamine;

and pharmaceutically acceptable salts thereof.

A number of preferred compounds of the invention, including some of those listed above, are additionally described in TABLE I:

| | HPLC | MS | |
|---|---|---|---|
| | [min] | [M + H+] | IUPAC Name |
| 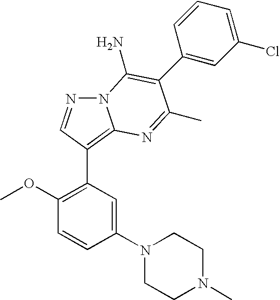 | | | |
| 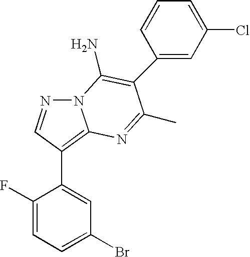 | | | |
| 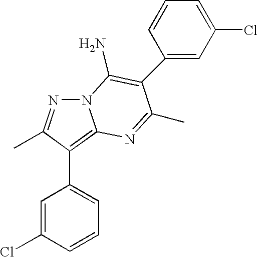 | | | |
| 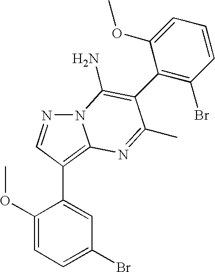 | | | |
| 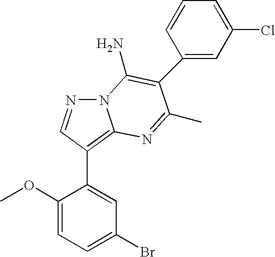 | | | |
| 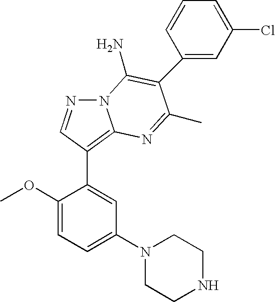 | 0.92 | 449 | 6-(3-Chloro-phenyl)-3-(2-methoxy-5-piperazin-1-yl-phenyl)-5-methyl-pyrazolo[1,5-a]pyrimidin-7-ylamine |
| 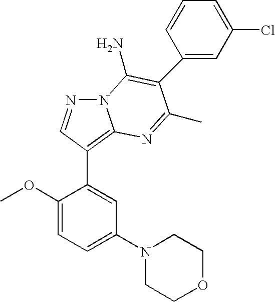 | 0.95 | 450 | 6-(3-Chloro-phenyl)-3-(2-methoxy-5-morpholin-4-yl-phenyl)-5-methyl-pyrazolo[1,5-a]pyrimidin-7-ylamine |
| 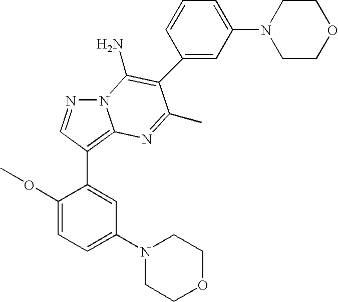 | 0.84 | 501 | 3-(2-Methoxy-5-morpholin-4-yl-phenyl)-5-methyl-6-(3-morpholin-4-yl-phenyl)-pyrazolo[1,5-a]pyrimidin-7-ylamine |
| 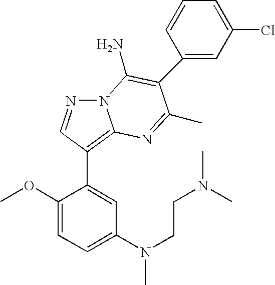 | 0.94 | 465 | N-{3-[7-Amino-6-(3-chloro-phenyl)-5-methyl-pyrazolo[1,5-a]pyrimidin-3-yl]-4-methoxy-phenyl}-N,N′,N′-trimethyl-ethane-1,2-diamine |
| 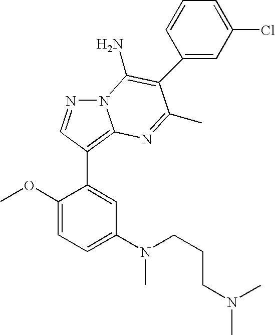 | 0.84 | 479 | N-{3-[7-Amino-6-(3-chloro-phenyl)-5-methyl-pyrazolo[1,5-a]pyrimidin-3-yl]-4-methoxy-phenyl}-N,N′,N′-trimethyl-propane-1,3-diamine |
| 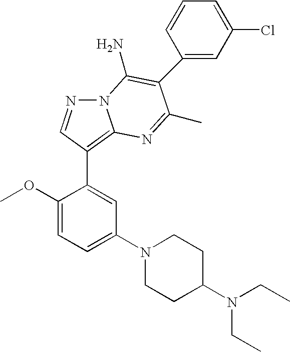 | 0.91 | 519 | 6-(3-Chloro-phenyl)-3-[5-(4-diethylamino-piperidin-1-yl)-2-methoxy-phenyl]-5-methyl-pyrazolo[1,5-a]pyrimidin-7-ylamine |
| 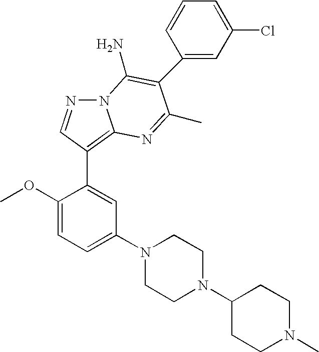 | 0.97 | 546 | 6-(3-Chloro-phenyl)-3-{2-methoxy-5-[4-(1-methyl-piperidin-4-yl)-piperazin-1-yl]-phenyl}-5-methyl-pyrazolo[1,5-a]pyrimidin-7-ylamine |
| 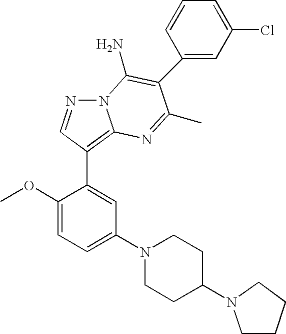 | 0.91 | 517 | 6-(3-Chloro-phenyl)-3-[2-methoxy-5-(4-pyrrolidin-1-yl-piperidin-1-yl)-phenyl]-5-methyl-pyrazolo[1,5-a]pyrimidin-7-ylamine |
| 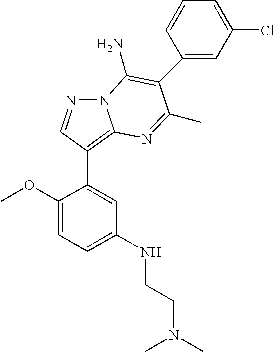 | 0.93 | 451 | N-{3-[7-Amino-6-(3-chloro-phenyl)-5-methyl-pyrazolo[1,5-a]pyrimidin-3-yl]-4-methoxy-phenyl}-N′,N′-dimethyl-ethane-1,2-diamine |
| 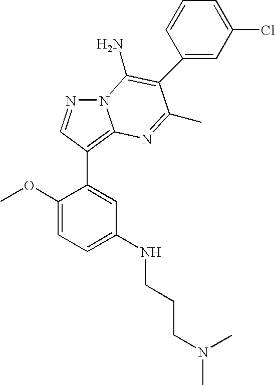 | n.d. | 465 | N-{3-[7-Amino-6-(3-chloro-phenyl)-5-methyl-pyrazolo[1,5-a]pyrimidin-3-yl]-4-methoxy-phenyl)-N′,N′-dimethyl-propane-1,3-diamine |
| 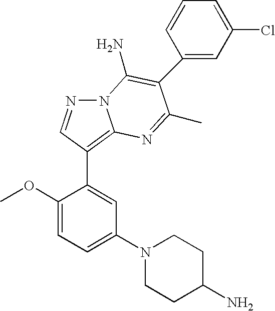 | n.d. | 463 | 3-[5-(4-Amino-piperidin-1-yl)-2-methoxy-phenyl]-6-(3-chloro-phenyl)-5-methyl-pyrazolo[1,5-a]pyrimidin-7-ylamine |
| 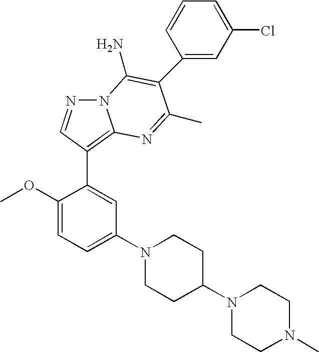 | 0.86 | 546 | 6-(3-Chloro-phenyl)-3-{2-methoxy-5-[4-(4-methyl-piperazin-1-yl)-piperidin-1-yl]-phenyl}-5-methyl-pyrazolo[1,5-a]pyrimidin-7-ylamine |
| 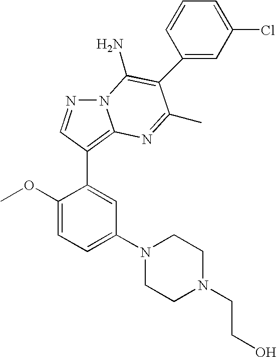 | 0.92 | 493 | 2-(4{3-[7-Amino-6-(3-chloro-phenyl)-5-methyl-pyrazolo[1,5-a]pyrimidin-3-yl]-4-methoxy-phenyl}-piperazin-1-yl)-ethanol |
| 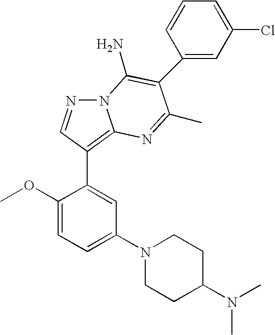 | 0.89 | 491 | 6-(3-Chloro-phenyl)-3-[5-(4-dimethylaminopiperidin-1-yl)-2-methoxy-phenyl]-5-methyl-pyrazolo[1,5-a]pyrimidin-7-ylamine |
| 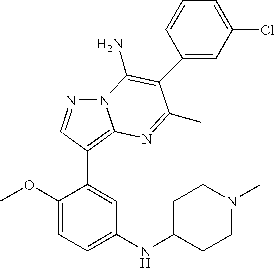 | 0.9 | 477 | 6-(3-Chloro-phenyl)-3-[2-methoxy-5-(1-methyl-piperidin-4-ylamino)-phenyl]-5-methyl-pyrazolo[1,5-a]pyrimidin-7-ylamine |
| 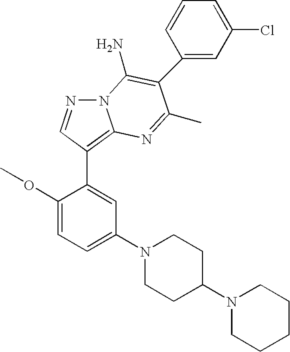 | n.d. | 531 | 3-(5-[1,4′]Bipiperidinyl-1′-yl-2-methoxy-phenyl)-6-(3-chloro-phenyl)-5-methyl-pyrazolo[1,5-a]pyrimidin-7-ylamine |
| 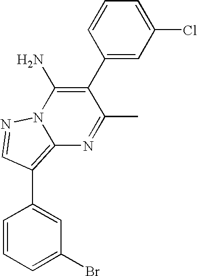 | | | |
| 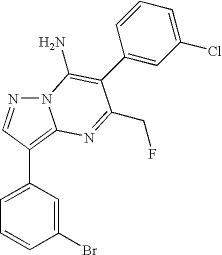 | 3.94 | 431 | 3-(3-Bromo-phenyl)-6-(3-chloro-phenyl)-5-fluoromethyl-pyrazolo[1,5-a]pyrimidin-7-ylamine |
| 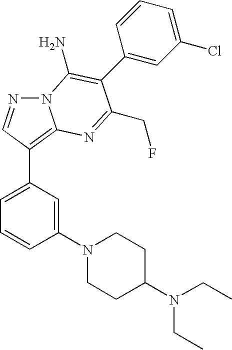 | 2.4 | 508 | 6-(3-Chloro-phenyl)-3-[3-(4-diethylamino-piperidin-1-yl)-phenyl]-5-fluoromethyl-pyrazolo[1,5-a]pyrimidin-7-ylamine |
| 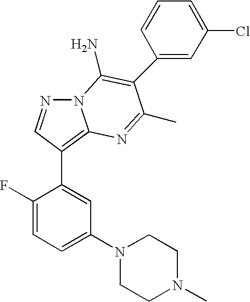 | 0.78 | 451 | 6-(3-Chloro-phenyl)-3-[2-fluoro-5-(4-methyl-piperazin-1-yl)-phenyl]-5-methyl-pyrazolo[1,5-a]pyrimidin-7-ylamine |
| 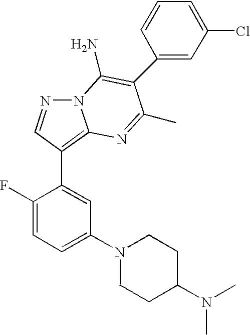 | 0.78 | 479 | 6-(3-Chloro-phenyl)-3-[5-(4-dimethylamino-piperidin-1-yl)-2-fluoro-phenyl]-5-methyl-pyrazolo[1,5-a]pyrimidin-7-ylamine |
| 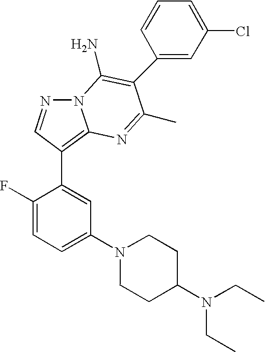 | 0.81 | 507 | 6-(3-Chloro-phenyl)-3-[5-(4-diethylamino-piperidin-1-yl)-2-fluoro-phenyl]-5-methyl-pyrazolo[1,5-a]pyrimidin-7-ylamine |
| 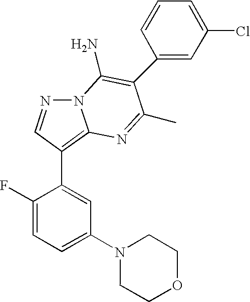 | 0.91 | 438 | 6-(3-Chloro-phenyl)-3-(2-fluoro-5-morpholin-4-yl-phenyl)-5-methyl-pyrazolo[1,5-a]pyrimidin-7-ylamine |
| 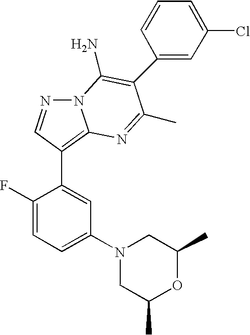 | 1.03 | 466 | 6-(3-Chloro-phenyl)-3-[5-((2R,6S)-2,6-dimethyl-morpholin-4-yl)-2-fluoro-phenyl]-5-methyl-pyrazolo[1,5-a]pyrimidin-7-ylamine |
| 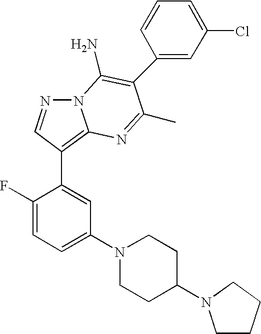 | 0.8 | 505 | 6-(3-Chloro-phenyl)-3-[2-fluoro-5-(4-pyrrolidin-1-yl-piperidin-1-yl)-phenyl]-5-methyl-pyrazolo[1,5-a]pyrimidin-7-ylamine |
| 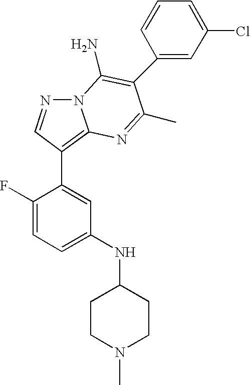 | 0.77 | 465 | 6-(3-Chloro-phenyl)-3-[2-fluoro-5-(1-methyl-piperidin-4-ylamino)-phenyl]-5-methyl-pyrazolo[1,5a]pyrimidin-7-ylamine |
| 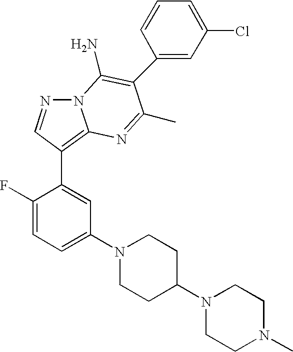 | 0.75 | 534 | 6-(3-Chloro-phenyl)-3-{2-fluoro-5-[4-(4-methyl-piperazin-1-yl)-piperidin-1-yl]-phenyl}-5-methyl-pyrazolo[1,5-a]pyrimidin-7-ylamine |
| 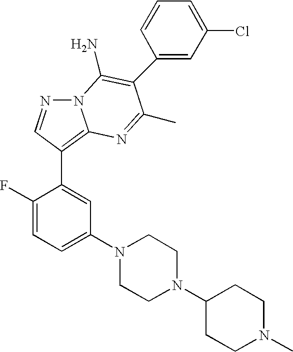 | 0.74 | 534 | 6-(3-Chloro-phenyl)-3-{2-fluoro-5-[4-(1-methyl-piperidin-4-yl)-piperazin-1-yl]-phenyl}-5-methyl-pyrazolo[1,5-a]pyrimidin-7-ylamine |
| 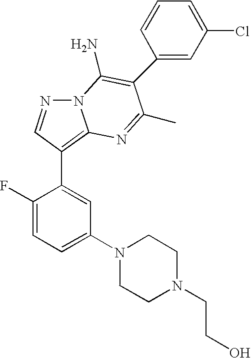 | 0.77 | 481 | 2-(4-{3-[7-Amino-6-(3-chloro-phenyl)-5-methyl-pyrazolo[1,5-a]pyrimidin-3-yl]-4-fluoro-phenyl}-piperazin-1-yl)-ethanol |
| 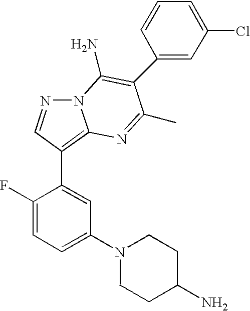 | 0.75 | 451 | 3-[5-(4-Amino-piperidin-1-yl)-2-fluoro-phenyl]-6-(3-chloro-phenyl)-5-methyl-pyrazolo[1,5-a]pyrimidin-7-ylamine |
| 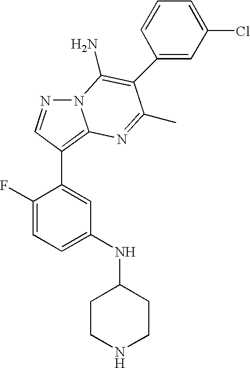 | n.d. | 451 | 6-(3-Chloro-phenyl)-3-[2-fluoro-5-(piperidin4-ylamino)-phenyl]-5-methyl-pyrazolo[1,5-a]pyrimidin-7-ylamine |
| 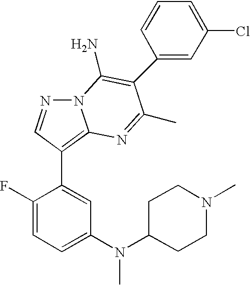 | n.d. | 479 | 6-(3-Chloro-phenyl)-3-{2-fluoro-5-[methyl-(1-methyl-piperidin-4-yl)-amino]-phenyl}-5-methyl-pyrazolo[1,5-a]pyrimidin-7-ylamine |
| 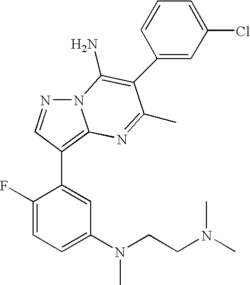 | 0.81 | 453 | N-{3-[7-Amino-6-(3-chloro-phenyl)-5-methyl-pyrazolo[1,5-a]pyrimidin-3-yl]-4-fluoro-phenyl}-N,N′,N′-trimethy-ethane-1,2-diamine |
| 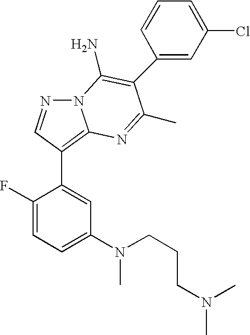 | 0.78 | 467 | N-{3-[7-Amino-6-(3-chloro-phenyl)-5-methyl-pyrazolo[1,5-a]pyrimidin-3-yl]-4-fluoro-phenyl]-N,N′,N′-trimethyl-propane-1,3-diamine |
| 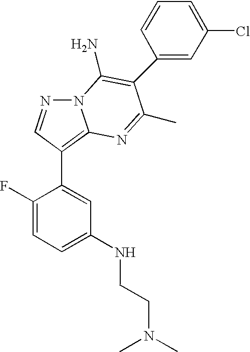 | 0.78 | 439 | N-{3-[7-Amino-6-(3-chloro-phenyl)-5-methyl-pyrazolo[1,5-a]pyrimidin-3-yl]-4-fluoro-phenyl}-N′,N′-dimethyl-ethane-1,2-diamine |
| 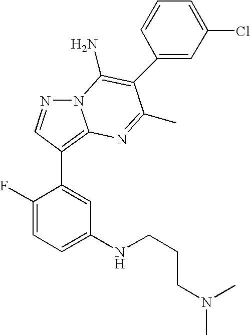 | 0.76 | 453 | N-[3-[7-Amino-6-(3-chloro-phenyl)-5-methyl-pyrazolo[1,5-a]pyrimidin-3-yl]-4-fluoro-phenyl}-N′,N′-dimethyl-propane-1,3-diamine |
| 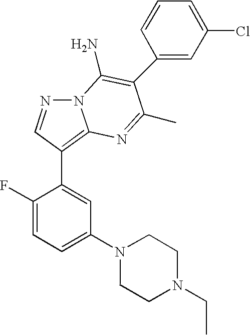 | 0.8 | 465 | 6-(3-Chloro-phenyl)-3-[5-(4-ethyl-piperazin-1-yl)-2-fluoro-phenyl]-5-methyl-pyrazolo[1,5-a]pyrimidin-7-ylamine |
| 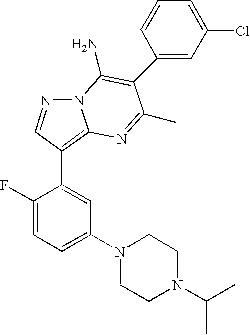 | 0.82 | 479 | 6-(3-Chloro-phenyl)-3-[2-fluoro-5-(4-isopropyl-piperazin-1-yl)-phenyl]-5-methyl-pyrazolol[1,5-a]pyrimidin-7-ylamine |
| 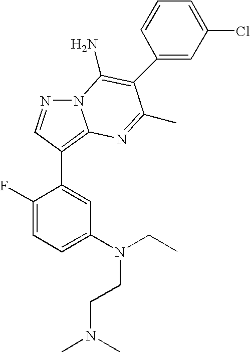 | 0.86 | 467 | N-{3-[7-Amino-6-(3-chloro-phenyl)-5-methyl-pyrazolo[1,5-a]pyrimidin-3-yl]-4-fluoro-phenyl}-N-ethyl-N′,N′-dimethyl-ethane-1,2-diamine |
| 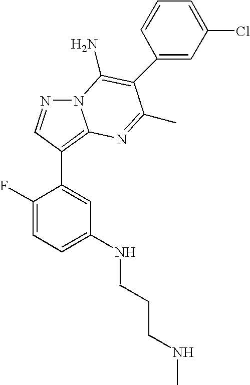 | n.d. | 439 | N-{3-[7-Amino-6-(3-chloro-phenyl)-5-methyl-pyrazolo[1,5-a]pyrimidin-3-yl]-4-fluoro-phenyl}-N′-methyl-propane-1,3-diamine |
| 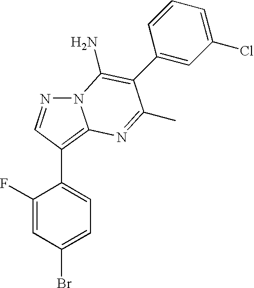 | | | |
| 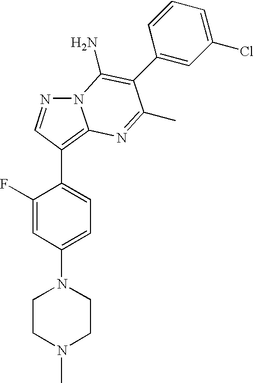 | 0.91 | 451 | 6-(3-Chloro-phenyl)-3-[2-fluoro-4-(4-methyl-piperazin-1-yl)-phenyl]-5-methyl-pyrazolo[1,5-a]pyrimidin-7-ylamine |
| 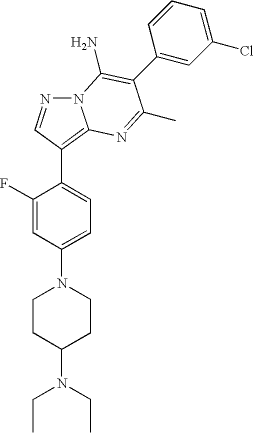 | 0.98 | 507 | 6-(3-Chloro-phenyl)-3-[4-(4-diethylamino-piperidin-1-yl)-2-fluoro-phenyl]-5-methyl-pyrazolo[1,5-a]pyrimidin-7-ylamine |
| | 1.16 | 438 | 6-(3-Chloro-phenyl)-3-(2-fluoro-4-morpholin-4-yl-phenyl)-5-methyl-pyrazolo[1,5-a]pyrimidin-7-ylamine |
| 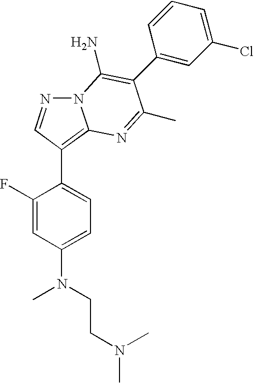 | 0.93 | 453 | N-{4-[7-Amino-6-(3-chloro-phenyl)-5-methyl-pyrazolo[1,5-a]pyrimidin-3-yl]-3-fluoro-phenyl}-N,N′,N′-trimethyl-ethane-1,2-diamine |
| 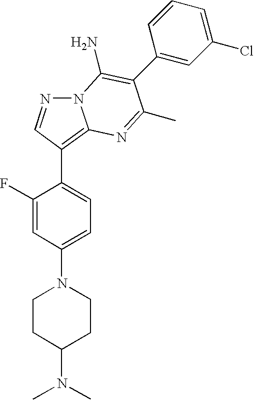 | 0.92 | 479 | 6-(3-Chloro-phenyl)-3-[4-(4-dimethylamino-piperidin-1-yl)-2-fluoro-phenyl]-5-methyl-pyrazolo[1,5-a]pyrimidin-7-ylamine |
| 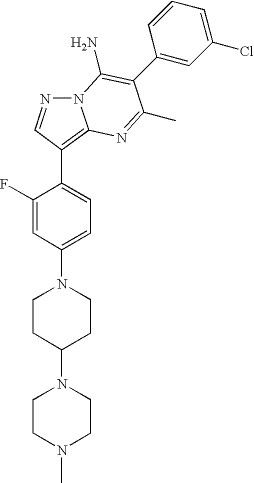 | 0.86 | 534 | 6-(3-Chloro-phenyl)-3-{2-fluoro-4-[4-(4-methyl-piperazin-1-yl)-piperidin-1-yl]-phenyl}-5-methyl-pyrazolo[1,5-a]pyrimidin-7-ylamine |
| 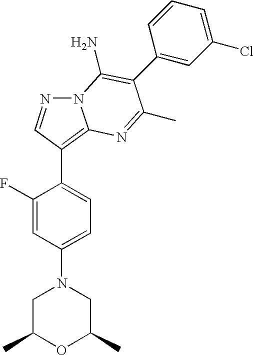 | 1.31 | 466 | 6-(3-Chloro-phenyl)-3-[4-((2R,6S)-2,6-dimethyl-morpholin-4-yl)-2-fluoro-phenyl]-5-methyl-pyrazolo[1,5-a]pyrimidin-7-ylamine |
| 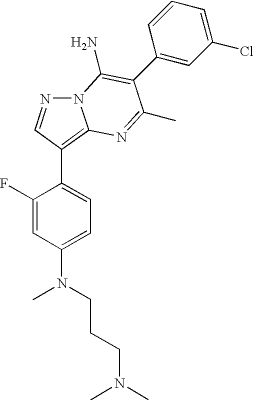 | 0.97 | 467 | N-{4-[7-Amino-6-(3-chloro-phenyl)-5-methyl-pyrazolo[1,5-a]pyrimidin-3-yl]-3-fluoro-phenyl-N,N′,N′-trimethyl-propane-1,3-diamine |

In an embodiment of the invention, the compounds of the invention are used in methods of treatment of Eph receptor-related (e.g., neurological) injuries and disorders

In another embodiment of the invention, pharmaceutical compositions prepared from the compounds of the invention are used in methods of treatment of Eph receptor-related (e.g., neurological) injuries and disorders. The pharmaceutical compositions preferably comprise a compound of the invention an acceptable pharmaceutical carrier. Carriers are described in greater details herein.

In another embodiment of the invention, the compounds of the invention are used to contact a cell, in order to modulate the activity of an Eph receptor therein. The cell can be contacted in vitro or in vivo, in an effective amount of the compounds of the invention to modulate Eph receptors therein.

In yet another embodiment of the invention, the compounds of the invention are used in methods of stimulating and promoting neural regeneration (such as axon regeneration), and reversing neuronal degeneration due to traumatic injury, stroke, multiple sclerosis and neurodegenerative diseases. One way in which this can be achieved is through the administration to a mammal of a compound of the invention in an amount that is sufficient to stimulate and promote neural regeneration (such as axon regeneration) or reverse neuronal degeneration. The compounds of the invention can be delivered to both normal and injured cells. In some embodiments, the compounds of the invention inhibit the phosphorylation of an Eph receptor. In other embodiments, the compounds of the invention inhibit the binding of ephrin ligands to Eph receptors.

In still yet another embodiment of the invention, the compounds of the invention are used in methods for delivering a therapeutic agent to a cell, such as via a conjugate comprising said therapeutic agent linked to compound of the invention. As described in greater detail herein, the therapeutic agent can be a linking reagent.

A further embodiment is a process to prepare a compound according to the above comprising:

reacting a nitrile, A-CH₂—C≡N, with ethyl formate in the presence of an organic solvent to form a substituted 3-oxo-propionitrile,

condensing the substituted 3-oxo-propionitriles of step (a) with hydrazine monohydrate in an organic solvent to form a 2H-pyrazol-3-ylamine of formula (III):

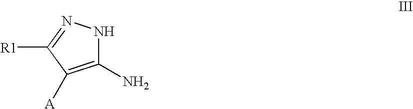

formylating a substituted nitrile in the presence of ethanolate and formic acid ethyl ester to prepare a 3-oxo-propionitrile of formula (II):

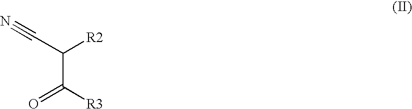

condensing the 3-oxo-propionitrile of formula (II) and the 2H-pyrazol-3-ylamines of formula (III) in the presence of an organic solvent to form a compound of formula (I).

The invention in particular relates to The present invention relates to compounds of the invention, including pyrazolo[1,5a]pyrimidin-7-yl amine compounds, of the formula (I):

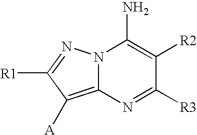

wherein:

R₂ is H; substituted or unsubstituted aryl; substituted or unsubstituted heteroaryl; substituted or unsubstituted aliphatic residue; a functional group; or a substituted or unsubstituted aryl, substituted or unsubstituted heteroaryl or substituted or unsubstituted aliphatic residue which is connected by one connecting group or atom to the pyrazolo[1,5a]pyrimidinyl ring;

R₃ is H, substituted or unsubstituted aryl, substituted or unsubstituted heteroaryl, substituted or unsubstituted aliphatic residue, a functional group, or an aliphatic residue which may be connected by a connecting group or atom to the pyrazolo[1,5a]pyrimidinyl ring, at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;

A is H, halogen (such as bromo), an aliphatic moiety, a functional group, substituted or unsubstituted aryl, or substituted or unsubstituted heteroaryl; and

R₁ is H, halogen or lower alkyl,

or pharmaceutically acceptable salts thereof,

in the treatment of Eph receptor-related (e.g., neurological) injuries and disorders or for the manufacture of pharmaceutical compositions for use in the treatment of said injuries and disorders, methods of use of compounds of formula (I) in the treatment of said injuries and disorders, or pharmaceutical preparations comprising compounds of formula (I) for the treatment of said injuries and disorders.

The present invention is especially related to a compound of formula (I) wherein R₂ is H; substituted or unsubstituted aryl; substituted or unsubstituted heteroaryl; substituted or unsubstituted aliphatic residue; a functional group; or a substituted or unsubstituted aryl, substituted or unsubstituted heteroaryl or substituted or unsubstituted aliphatic residue which is connected by one connecting group or atom to the pyrazolo[1,5a]pyrimidinyl ring;

R₃ is H, substituted or unsubstituted aryl, substituted or unsubstituted heteroaryl, substituted or unsubstituted aliphatic residue, a functional group, or an aliphatic residue which may be connected by a connecting group or atom to the pyrazolo[1,5a]pyrimidinyl ring,

at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring, and provided that R₂ and A cannot both be unsubstituted phenyl;

A is H, halogen (such as bromo), an aliphatic moiety, a functional group, substituted or unsubstituted aryl or heteroaryl; and

R₁ is H, halogen or lower alkyl, or pharmaceutically acceptable salts thereof,

in the treatment of Eph receptor-related (e.g., neurological) injuries and disorders or for the manufacture of pharmaceutical compositions for use in the treatment of said injuries and disorders, methods of use of compounds of formula (I) in the treatment of said injuries and disorders, pharmaceutical preparations comprising compounds of formula (I) for the treatment of said injuries and disorders, compounds of formula (I) for use in the treatment of said injuries and disorders.

The present invention also relates to a method of treating kinase dependent diseases comprising administering pyrazolo[1,5a]pyrimidin-7-yl amine compounds of the formula (I) to a warm-blooded animal, especially a human. The present invention also relates to pharmaceutical preparations comprising an pyrazolo[1,5a]pyrimidin-7-yl amine compound of the formula (I), especially for the treatment of a kinase dependent disease, novel pyrazolo[1,5a]pyrimidin-7-yl amine compounds of the formula (I), a process for the manufacture of the pyrazolo[1,5a]pyrimidin-7-yl amine compounds of the formula (I), and novel starting materials and intermediates for their manufacture. The present invention also relates to use of a compound of formula I in the manufacture of a pharmaceutical preparation for the treatment of an Eph receptor-related (e.g., neurological) injury or disorder.

### DEFINITIONS

The general terms used hereinbefore and hereinafter preferably have within the context of this disclosure the following meanings, unless otherwise indicated.

The following abbreviations are used herein to represent commonly used terms (in parenthesis) in the present application, including but not limited to the examples section: DMSO (dimethylsulfoxide); ES-MS (electrospray mass spectrometry); EtOAc (Ethyl Acetate); HPLC (high-pressure liquid chromatography); mL (mililiter(s)); NMR (nuclear magnetic resonance); RT (room temperature); ^{A}t_{RET} (HPLC retention time in minutes (method A)); ^{B}t_{RET} (HPLC retention time in minutes (method B)); ^{C}t_{RET} (HPLC retention time in minutes (method C)); ^{D}t_{RET} (HPLC retention time in minutes (method D)); TFA (trifluoroacetic acid); THF (tetrahydrofuran); TMSCl (Trimethylsilyl chloride).

As used herein, “Eph receptor” means a receptor tyrosine kinase that belongs to the Eph family, including EphA2, EphA4, EphA5, EphA7, EphB2 and EphB4. This family is reviewed, for instance, in Pasquale, E. (1997) Curr. Opin. Cell Biol. 9:608-615; and Orioli and Klein (1997) Trends in Genetics 13:354-359.

As used herein, the term “treatment” includes both prophylactic or preventive treatment as well as curative or disease suppressive treatment, including treatment of patients at risk of neurological disorders, as well as ill and injured patients. This term further includes the treatment for the delay of progression of the disease.

“Eph receptor-related injuries and disorders” include neurological injuries and disorders, including but not limited to spinal cord injury (SCI); quadriplegia, hemiplegia, and paraplegia, including injury-caused and hereditary forms; neuropathies; CNS related disorders (e.g., bacterial and viral meningitis); and neurodegenerative disorders (e.g., Alzheimers Disease, cerebral toxoplasmosis, Parkinson's disease, amytropic lateral sclerosis (ALS), and multiple sclerosis).

“Eph-receptor-related injuries and disorders” also includes neuronal degeneration resulting from hypoxic conditions, or from an infarct as in stroke. This condition can result in deficits in motor, sensory and cognitive functions, in large part due to the inability of injured axons to regenerate and undergo synaptic reorganization. As with SCI, stroke is followed by the formation of a glial scar at the site of infarction, and inhibiting EphA4 (e.g., as with the compounds of the invention) can inhibit scarring and thus enable improved regeneration and reorganization of connections. An in vitro model of stroke using astrocyte-hippocampal neuron co-cultures, has been shown that the inter-astrocytic gap-junctions are very important for the survival of neurons following hypoxic stress. (Blanc, E. M., et al. (1998) J Neurochem, 70(3): 958). As Eph receptors are known to be involved in signaling at gap-junctions, this represents another potential impliocation of EphA4 in ischemic stroke (Mellitzer, G., et al. (1999) Nature, 400(6739): 77).

As used herein, “compounds of the invention” include, compounds of formula (I), including pyrazolo[1,5a]pyrimidin-7-yl amine derivatives. Compounds of the invention also refers to those compounds referred to herein as “Compound [number].”

“Aryl” is an aromatic radical having 6 to 14 carbon atoms, especially phenyl, naphthyl, indenyl, azulenyl, or anthryl, and is unsubstituted or substituted by one or more, preferably one or two substituents, wherein the substituents are selected from any of the functional groups defined below, and including: lower halo, alkyl, substituted alkyl, halo lower alkyl e.g. trifluoromethyl, lower alkenyl, lower alkynyl, lower alkanoyl, lower alkoxy, hydroxy, etherified or esterified hydroxy, amino, mono- or disubstituted amino, amino lower alkyl, amino lower alkoxy; acetyl amino; amidino, halogen, nitro, cyano, cyano lower alkyl, carboxy, esterified carboxy especially lower alkoxy carbonyl, e.g. methoxy carbonyl, n-propoxy carbonyl or iso-propoxy carbonyl, alkanoyl, benzoyl, carbamoyl, N-mono- or N,N-disubstituted carbamoyl, carbamates, alkyl carbamic acid esters, amidino, guanidino, urea, ureido, mercapto, sulfo, lower alkylthio, sulfoamino, sulfonamide, benzosulfonamide, sulfonate, phenyl, benzyl, phenoxy, benzyloxy, phenylthio, phenyl-lower alkylthio, alkylphenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, halogen-lower alkylmercapto, halogen-lower alkylsulfonyl, such as especially trifluoromethane sulfonyl, dihydroxybora (—B(OH)₂); heterocyclyl, and lower alkylene dioxy bound at adjacent C-atoms of the ring, such as methylene dioxy, phosphono (—P(═O)(OH)₂), hydroxy-lower alkoxy phosphoryl or di-lower alkoxyphosphoryl, carbamoyl, mono- or di-lower alkylcarbamoyl, mono- or di-(hydroxy-lower alkyl)-carbamoyl, or —NR₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl, lower alkyl-piperazinyl, azetidinyl, pyrrolidinyl, piperidino, morpholinyl, imidazolinyl).

Aryl is more preferably phenyl which is either unsubstituted or independently substituted by one or two substituents selected from a solubilizing group selected from the group consisting of: halo (such as Cl or Br); hydroxy; lower alkyl (such as C₁-C₃ lower alkyl); aryl (such as phenyl or benzyl); amino; amino lower alkyl (such as dimethylamino); acetyl amino; amino lower alkoxy (such as ethoxyamine); lower alkyl (such as methyl); alkoxy (such as methoxy or benzyloxy where the benzyl ring may be substituted or unsubstituted, such as 3,4-dichlorobenzyloxy); sulfoamino; substituted or unsubstituted sulfonamide (such as benzo sulfonamide, chlorobenzene sulfonamide or 2,3-dichloro benzene sulfonamide); substituted or unsubstituted sulfonate (such as chloro-phenyl sulfonate); substituted urea (such as 3-trifluoro-methyl-phenyl urea or 4-morpholin-4-yl-3-trifluoromethyl-phenyl-urea); alkyl carbamic acid ester or carbamates (such as ethyl-N-phenyl-carbamate) or —NR₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 14 nitrogen, oxygen or sulfur atoms (e.g. piperazinyl, lower alkyl-piperazinyl, pyridyl, indolyl, thiophenyl, thiazolyl, morpholinyl n-methyl piperazinyl, benzothiophenyl, azetidinyl, pyrrolidinyl, piperidino or imidazolinyl);

A heteroaryl group is preferably monocyclic, but may be bi- or tri-cyclic, and comprises 3-24, preferably 4-16 ring atoms, wherein at least one or more, preferably one to four ring carbons are replaced by a heteroatom selected from O, N or S. Preferably the heteroaryl group is selected from pyridyl, indolyl, pyrimidyl, pyrazolyl, oxazolyl, thiophenyl, benzothiophenyl, 2H-pyrrolyl, pyrrolyl, imidazolyl, benzimidazolyl, pyrazolyl, indazolyl, purinyl, pyrazinyl, pyridazinyl, 4H-quinolizinyl, isoquinolyl, quinolyl, phthalazinyl, naphthyridinyl, quinoxalyl, quinazolinyl, quinnolinyl, indolizinyl, 3H-indolyl, isoindolyl, isoxazolyl, thiazolyl, isothiazolyl, triazolyl, tetrazolyl, furazanyl and benzo[d]pyrazol.

More preferably the heteroaryl group is selected from the group consisting of pyridyl, indolyl, pyrimidyl, pyrazolyl, oxazolyl, thiophenyl or benzothiophenyl.

The heteroaryl group may be unsubstituted or substituted by one or more substituents selected from the group defined above as substituents for aryl, most preferably by hydroxy, halogen, lower alkyl, such as methyl or lower alkoxy, such as methoxy or ethoxy.

“Aliphatic,” as used herein, refers to any non-aromatic carbon based residue. Examples of aliphatic-residues include substituted or unsubstituted alkyl, cycloalkyl, alkenyl and alkynyl.

“Alkyl” includes lower alkyl preferably alkyl with up to 7 carbon atoms, preferably from 1 to and including 5, and is linear or branched; preferably, lower alkyl is pentyl, such as n-pentyl, butyl, such as n-butyl, sec-butyl, isobutyl, tert-butyl, propyl, such as n-propyl or isopropyl, ethyl or methyl. Preferably lower alkyl is methyl, propyl or tert-butyl.

A cycloalkyl group is preferably cyclopentyl, cyclohexyl or cycloheptyl, and may be unsubstituted or substituted by one or more, especially one or two, substituents selected from the group defined above as substituents for aryl, most preferably by lower alkyl, such as methyl, lower alkoxy, such as methoxy or ethoxy, or hydroxy.

Alkenyl and alkynyl preferably have up to 7 carbon atoms, preferably from 1 to and including 5, and can be linear or branched.

Alkyl, cycloalkyl, alkenyl and alkynyl can be substituted or unsubstituted, and when substituted may be with up to 3 substituents including other alkyl, cycloalkyl, alkenyl, alkynyl, any of the substituents defined above for aryl or any of the functional groups defined below.

“Halo” or “halogen” is preferably fluoro, chloro, bromo or iodo, most preferably fluoro, chloro or bromo.

The term “connecting atom or group” as used herein includes alkyl, (such, as —CH₂—); oxy —O—; keto —CO—; thio —S—; sulfonyl —SO₂—; sulfoxides —SO—; amines —NH— or —NR—; carboxylic acid; alcohol; esters (—COO—); amides (—CONR—, —CONHR′—); sulfonamides (—SO₂NH—, —SO₂NR′—); sulfones (—SO₂—); sulfoxides (—SO—); amino-group; ureas (—NH—CO—NH—, —NR—CO—NH—, —NH—CO—NR—, —NR—CO—NR—); ethers (—O—); carbamates (—NH—CO—O—, —NR—CO—O—); or inverse amides sulfonamides and esters (—NH—CO—, —NR—CO—, —NH—SO₂—, —NR—SO₂—, —OOC—).

The term “functional group” as used herein includes: carboxylic acid; hydroxyl; halogen; cyano (—CN); ethers (—OR); ketones (—CO—R); esters (—COOR); amides (—CONH2, —CONHR, —CONRR′); thioethers (—SR); sulfonamides (—SO₂NH₂, —SO₂NHR, —SO₂NRR′); sulfones (—SO₂—R); sulfoxides (—SO—R); amines (—NHR, NR′R); ureas (—NH—CO—NH₂, —NH—CO—NHR); ethers (—O—R); halogens; carbamates (—NH—CO—OR); aldehyde-function (—CHO); then also inverse amides; sulfonamides and esters (—NH—CO—R, —NH—SO₂—R, —OOC—R);

R and R′ are the same are different and may be H or are any aliphatic, aryl or heteroaryl moiety as defined above.

Where the plural form is used for compounds, salts, pharmaceutical preparations, diseases and the like, this is intended to mean also a single compound, salt, or the like.

Salts are especially the pharmaceutically acceptable salts of compounds of formula (I).

Such salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula (I) may also form internal salts.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the close relationship between the compounds in free form and those in the form of their salts, including those salts that can be used as intermediates, for example in the purification or identification of the compounds, tautomers or tautomeric mixtures and their salts, any reference to the compounds hereinbefore and hereinafter especially the compounds of the formula (I), is to be understood as referring also to the corresponding tautomers of these compounds, especially of compounds of the formula (I), tautomeric mixtures of these compounds, especially of compounds of the formula (I), or salts of any of these, as appropriate and expedient and if not mentioned otherwise.

Where “a compound . . . , a tautomer thereof; or a salt thereof” or the like is mentioned, this means “a compound . . . , a tautomer thereof, or a salt of the compound or the tautomer.”

Any asymmetric carbon atom may be present in the (R)-, (S)- or (R,S)-configuration, preferably in the (R)- or (S)-configuration. Substituents at a ring at atoms with saturated bonds may, if possible, be present in cis-(=Z-) or trans (=E-) form. The compounds may thus be present as mixtures of isomers or preferably as pure isomers, preferably as enantiomer-pure diastereomers or pure enantiomers.

The compounds of formula (I) have valuable pharmacological properties and are useful in the treatment of kinase dependent diseases, e.g., as drugs to treat neurological diseases.

The compounds of formula (I) have valuable pharmacological properties and are useful in the treatment of Eph receptor-related (e.g., neurological) injuries and disorders, e.g., as drugs to treat neurological diseases.

CNS-Related Injuries and Disorder

Injury to the central nervous system usually results in very limited, if any, regeneration of lesioned axons, with subsequent permanent impairment of function. Although some CNS neurons appear to lose the intrinsic ability to regenerate neurites postnatally, many others, such as corticospinal tract (CST) neurons, appear able to regenerate, but are inhibited from doing so by the environment of the injury site. (Goldberg et al., (2002) Science 296: 1860). Major impediments to CNS regeneration are the presence of myelin inhibitors and astrocytic gliosis.

Axonal regeneration is prevented by a host of inhibitory influences in the adult CNS, among them inhibitory myelin proteins and the formation of a glial scar. Although considerable progress has been made in identifying molecules associated with myelin inhibition (e.g., Nogo, myelin-associated glycoprotein (MAG), and oligodendrocyte-myelin glycoprotein (OMgp)), targeting those proteins for the treatment or amelioration of neurological disorders is an incomplete solution. Blocking individual myelin proteins or their common receptor in vivo after spinal cord injury can result in partial axon regeneration, and a concomitant improvement of functional recovery; however, only a small percentage of axons regrow, highlighting the need for the removal of other impediments to regeneration for a more complete therapeutic solution (Simonen M., et al. (2003) Neuron 38: 201; Zheng B., et al. (2003) Neuron 38: 213).

The main component of glial scarring is astrocytic gliosis, whereby normally quiescent astrocytes show a vigorous response to injury. (Stichel C C, et al. (1998) Cell Tissue Res 294: 1). They become hypertrophic, proliferative, upregulate expression of glial fibrillary acidic protein (GFAP), and form a dense network of glial processes both at and extending from the lesion site. At the same time, the astrocytes secrete a variety of cytokines and produce cell adhesion and extracellular matrix molecules, some of which are inhibitory to regeneration (e.g., chondroitin sulfate proteoglycan (CSPG) and collagen IV). Blocking the deposition of said astrocytic products can promote axonal regeneration is promoted. (Stitchel C C, et al.).

As most spinal cord injury-related attempted therapeutics to date have centered on overcoming either myelin inhibitors or components of the glial scar, agents aimed at inhibiting Eph receptors (e.g., the compounds of the invention) are representative of a new strategy to promote nerve regeneration.

Eph Receptors and Ephrins

The Eph receptor tyrosine kinase subfamily appears to be the largest subfamily of transmembrane receptor tyrosine kinases, and with its ligands, the ephrins, is responsible for governing proper cell migration and positioning during neural development, presumably through modulating intercellular repulsion (Pasquale, E. (1997) Curr. Opin. Cell Biol. 9:608-615)(Orioli and Klein (1997) Trends in Genetics 13:354-359). The Eph family is responsible for the formation of the corticospinal tract and anterior commissure. (Kullander K., et al. (2001a) Neuron 29: 73; Herikemeyer M, et al. (1996) Cell 86: 35).

Eph receptors are closely related, and actively signal when bound to their ephrin ligands (their effects are mediated by cell-to-cell contacts), with which they are capable of both forward and bi-directional signaling. (Murai, K. K., et al. (2003) J Cell Sci. 116(14): 2823).

These receptors are characterized by 3 functional domains: an intracellular tyrosine kinase catalytic domain, a single membrane spanning domain, and an extracellular ligand binding domain.

Binding of a ligand ephrin by a Eph receptor induces phosphorylation on tyrosine residues, which establishes binding sites for signaling proteins containing SH2 domains and activates an array of signaling pathways. The ephrins are thought to activate Eph receptors by clustering them and inducing autophosphorylation, while soluble monomeric ephrins are thought to inhibit Eph receptor activation. (Davis et al. (1994) Science 266: 816).

The sixteen known Eph receptors are divided into two subgroups (Epic and EphB) based on sequence homology. EphA receptors preferentially bind the glycosylphosphatidylinositol (GPI)-linked ephrin-A ligands, while EphB preferentially receptors bind the transmembrane ephin-B ligands. However, the ephrin ligands are rather promiscuous, and tend to lack selectivity in their activation of Eph receptors. (Murai, K. et al. (2003) Molecular and Cellular Neuroscience 24:1000). For instance, EphA4 can bind (and is therefore activated by) ligand ephrins B2 and B3, in addition to members of the ephrin A ligand family.

Eph receptor family members and their ephrin ligands are of interest as targets for therapy for the treatment of neurological disorders and injuries, including as targets for the promotion of axon regeneration, based on findings in the literature. For instance, because Eph-ephrin signaling appears to regulate axon guidance through contact repulsion, inducing the collapse of neuronal growth cones (Wahl S., et al. (2000) J Cell Biol 149: 263; Kullander et al.), and members of this family are upregulated in the adult after neural injury (Moreno-Flores M T, et al. (1999) Neuroscience 91: 193; Willson C A, et al. (2002) Cell Transplant 11: 229), the aberrant expression or absence of Eph receptors could prove pivotal in determining the outcome of injury in the adult CNS.

EphA4

EphA4 is a receptor tyrosine kinase from the EphA family which has important functions in the developing and adult nervous-system. Along with its known expression pattern during neural development (Mori, T., et al. (1995) Brain Res Mol Brain Res 29:325; Ohta, K., et al. (1996) Mechanisms of Development 54:59; Soans, C., et al. (1994) Oncogene 9:3353), EphA4 is expressed in brain regions that show extensive synaptic remodeling (Murai, K., et al. (2003) Nature Neurosci 6:153). In the adult, EphA4 is enriched in the hippocampus and cortex, two brain structures critical for learning and memory. The receptor is also enriched in migrating neural crest cells, growing axonal projections, and mature brain structures that show extensive plasticity. (Murai, et al.).

A recent study implicates EphA4 in two critical aspects of spinal cord injury, axonal inhibition and astrocytic gliosis. (Goldshmit, Y., et al. (2004) J Neurosci. 24(45): 10064). Goldshmit compared neural regeneration after spinal cord hemisection in wild-type and EphA4−/− mice, and discovered an overall functional improvement in the latter, characterized by a lack of astrocytic gliosis and regeneration of ipsilateral axons. Regarding the mechanisms through which improvements were seen, the experiments (as well as literature) demonstrates three roles for Eph receptors in axonal regeneration:

The first, as demonstrated by in vitro assays, is the direct inhibition of neurite outgrowth mediated by EphA4 on the astrocytes binding to a receptor-ligand on the axon. Such an action of EphA4 may provide a mechanism for the inhibition of neurite outgrowth on astrocytes observed in the presence of IFN, which Goldshmit has shown upregulates EphA4 expression. (Fok-Seang J., et al. (1998) Eur J Neurosci 10: 2400). These results suggest that EphA4 is yet another directly inhibitory molecule produced during astrocytic gliosis, in addition to other inhibitory components, such as extracellular matrix and myelin-derived molecules.

The second, and lesser-observed, mechanism may be by activation of EphA4 on the regenerating axons, similar to on E16 cortical neurons. However, EphA4 was found to be highly expressed only on astrocytes and motor neurons, and present at low levels on descending axons in lesioned adult spinal cord.

The third mechanism by which EphA4 exerts an inhibitory effect involves its vital role in activating astrocytes, leading to gliosis and the formation of a glial scar. Such activation appears to be dependent on responsiveness to cytokine stimulation and may be dependent on Rho activation. This cytokine-induced response may be attributable to the upregulation of EphA4 receptor expression on the astrocytes, allowing enhanced ligand binding and receptor activation. It is also possible that the cytokine-induced astrocyte proliferation and hypertrophy may be caused by transactivation of EphA4, as has been shown for FGF2- and PDGF-induced phosphorylation of EphrinB molecules (Chong et al., (2000) Mol Cell Biol 20: 724), leading to Rho activation and cytoskeletal rearrangement. The difference in glial activation seems to be astrocyte specific as there was no apparent difference in macrophage-microglial activation. Ephs and Ephrins have been reported to play a role in interactions between astrocytes and meningeal fibroblasts, excluding fibroblasts from the glial scar. (Bundesen L Q, et al. (2003) J Neurosci 23: 7789).

Synthetic Procedure

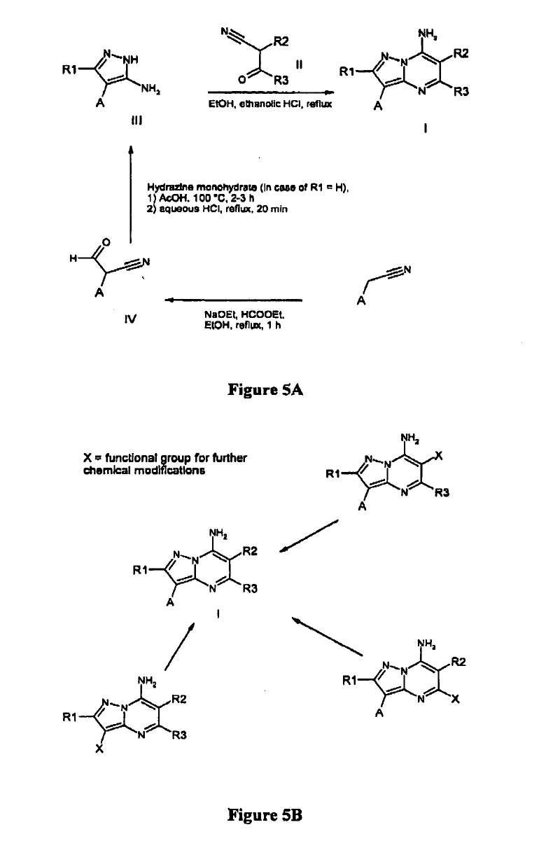

As seen in FIG. 5A, compounds of formula (I) are prepared analogously to the procedure described by Alicade, E; De Mendoza, J; Garcia-Marquina, J M; Almera, C; J. Heterocycl. Chem. 11, 423 (1974) by:

(a) reacting a nitrile, A-CH₂—C≡N, with ethyl formate in the presence of an organic solvent to form a substituted 3-oxo-propionitrile,

(b) condensing the substituted 3-oxo-propionitriles of step (a) with hydrazine monohydrate in an organic solvent to form a 2H-pyrazol-3-ylamine of formula (III):

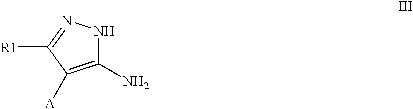

formylating a substituted nitrile in the presence of ethanolate and formic acid ethyl ester to prepare a 3-oxo-propionitrile of formula (II):

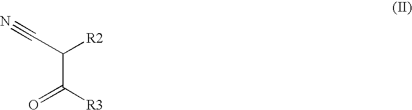

(c) condensing the 3-oxo-propionitrile of formula (II) and the 2H-pyrazol-3-ylamines of formula (III) in the presence of an organic solvent to form a compound of formula (I).

Specifically, compounds of formula (I) are prepared by condensing 3-oxo-propionitriles (II) and the corresponding 2H-pyrazol-3-ylamines (III) in the presence of ethanolic HCl. The 2H-pyrazol-3-ylamines (III) are prepared by condensing hydrazine monohydrate with the corresponding 3-oxo-propionitriles dissolved in an organic solvent, such as EtOH, dioxane or AcOH and heated at elevated temperatures (preferably at 100° C.) for several hours. The preferred procedure for preparing the pyrazolo moiety of the title compounds was stirring the hydrazine monohydrate with the corresponding 3-oxo-propionitriles in acetic acid at 100° C. for 2-3 h followed by addition of aqueous HCl and further refluxing the reaction mixture for further 20 min. In case where R1 is not H, the corresponding substituted hydrazines are used. The 3-oxo-propionitriles (I) and (II) are synthesized from the corresponding nitrites by classical formylation reaction using freshly prepared sodium ethanolate and formic acid ethyl ester (refluxing for 1 h in EtOH). Alternatively, instead of performing the condensation reactions with the 3-oxo-propionitiles, the corresponding 3,3-dialkoxy-propionitiles (in analogy to the procedure described by Seneci, P., Nicola, M., Inglesi, M., Vanotti, E., Resnati, G. Synth. Commun. 29 (2), 311-341 (1999)) or 3-dimethylamino-acrylonitriles can be used.

Alternatively, as seen in FIG. 5B, compounds of formula (I) can be prepared by first synthesizing the pyrazolo[1,5-a]pyrimidin-7-ylamine core scaffold carrying a corresponding functional group X, where residues A, R2, or R3, respectively, can be introduced by known reactions.

R₁, R₂, R₃, and X of FIG. 5B are as defined for compounds of the formula (I),

and, if desired, after reaction (a), (b) or (c), transforming an obtainable compound of formula (I) into a different compound of formula (I); transforming a salt of an obtainable compound of formula (I) into the free compound or a different salt or an obtainable free compound of formula (I) into a salt; and/or separating an obtainable mixture of isomers of compounds of formula (I) into the individual isomers;

where for all reactions mentioned functional groups in the starting materials that shall not take part in the reaction are, if required, present in protected form by readily removable protecting groups, and any protecting groups are subsequently removed.

The following reaction conditions are preferred, respectively:

Within the scope of this text, only a readily removable group that is not a constituent of the particular desired end product of formula (I) is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/1, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of formula (I) having at least one salt-forming group may be prepared in a manner known per se. For example, salts of compounds of formula (I) having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula (I) are obtained in customary manner, e.g. by treating the compounds with an acid or a suitable anion exchange reagent. Internal salts of compounds of formula (I) containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

General Process Conditions

The following applies in general to all processes mentioned hereinbefore and hereinafter, while reaction conditions specifically mentioned above or below are preferred:

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional-process steps.”

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitrites, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in new compounds of formula (I) described at the beginning as being especially valuable.

### PREFERRED EMBODIMENTS ACCORDING TO THE INVENTION

In the following preferred embodiments, general expression can be replaced by the corresponding more specific definitions provided above and below, thus yielding stronger preferred embodiments of the invention.

Preferred is the use of compounds of the formula (I), tautomers thereof or pharmaceutically acceptable salts thereof, where the Eph receptor-related (e.g., neurological) injuries and disorder to be treated is a neurological disorder or injury depending on Ephrin receptor kinases (e.g., EphA4 kinase).

The invention relates especially to use of a compound of the formula (I),

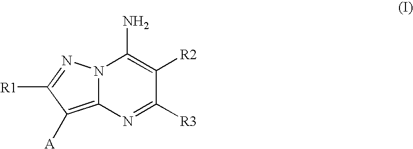

wherein:

R₂ is H, substituted or unsubstituted aryl, substituted or unsubstituted heteroaryl, substituted or unsubstituted aliphatic residue, a functional group, or a substituted or unsubstituted aryl, substituted or unsubstituted heteroaryl or substituted or unsubstituted aliphatic residue which is connected by one connecting group or atom to the pyrazolo[1,5a]pyrimidinyl ring;

R₃ can be H, substituted or unsubstituted aryl, substituted or unsubstituted heteroaryl, substituted or unsubstituted aliphatic residue, a functional group, or an aliphatic residue which may be connected by a connecting group or atom to the pyrazolo[1,5a]pyrimidinyl ring,

at least one of R2 or R3 is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;

A is H, halogen (such as bromo), an aliphatic moiety, a functional group, substituted or unsubstituted aryl or heteroaryl; and

R₁ is H, halogen or lower alkyl,

or pharmaceutically acceptable salts thereof,

and use of compounds of formula (I) in the treatment of kinase dependent diseases or for the manufacture of pharmaceutical preparations for the treatment of kinase dependent diseases.

The invention further relates to use of a compound of the formula (I),

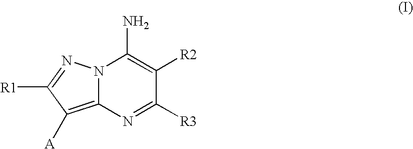

wherein:

R₂ is H, substituted or unsubstituted aryl, substituted or unsubstituted heteroaryl, substituted or unsubstituted aliphatic residue, a functional group, or a substituted or unsubstituted aryl, substituted or unsubstituted heteroaryl or substituted or unsubstituted aliphatic residue which is connected by one connecting group or atom to the pyrazolo[1,5a]pyrimidinyl ring;

R₃ can be H, substituted or unsubstituted aryl, substituted or unsubstituted heteroaryl, substituted or unsubstituted aliphatic residue, a functional group, or a substituted or unsubstituted aliphatic residue which may be connected by a connecting group or atom to the pyrazolo[1,5a]pyrimidinyl ring,

at least one of R2 or R3 is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;

and provided that R₂ and A cannot both be unsubstituted phenyl;

A is H, halogen (such as bromo), an aliphatic moiety, a functional group, substituted or unsubstituted aryl or heteroaryl; and

R₁ is H, halogen or lower alkyl,

or pharmaceutically acceptable salts thereof,

and use of compounds of formula (I) in the treatment of kinase dependent diseases or for the manufacture of pharmaceutical preparations for the treatment of kinase dependent diseases.

More preferred is a compound of the formula (I), wherein

the connecting atom or group is selected from the group consisting of: alkyl, (such as —CH₂—); oxy —O—; keto —CO—; thio —S—; sulfonyl —SO₂—; sulfoxides —SO—; amines —NH— or —NR—, carboxylic-acid; alcohol; esters (—COO—); amides (—CONR—, —CONHR′—); sulfonamides (—SO₂NH—, —SO₂NR′—); (—SO₃—); sulfoxides (—SO—); amino-group; ureas (—NH—CO—NH—, —NR—CO—NH—, —NH—CO—NR—, —NR—CO—NR—); ethers (—O—); carbamates (—NH—CO—O—, —NR—CO—O—); or inverse amides sulfonamides and esters (—NH—CO—, —NR—CO—, —NH—SO₂—, —NR—SO₂—, —OOC—); with alkyl, (such as —CH₂—); oxy —O—; keto —CO—; sulfonyl —SO₂—; sulfonamides (—SO₂NH—, —SO₂NR′—); (—SO₃—); and ureas (—NH—CO—NH—, —NR—CO—NH—, —NH—CO—NR—, —NR—CO—NR—) being especially preferred,

and the functional group is selected from the group consisting of: carboxylic acid; hydroxyl; halogens; cyano (—CN); ethers (—OR); ketones (—CO—R); esters (—COOR); amides (—CONH₂, —CONHR, —CONRR′); thioethers (—SR); sulfonamides (—SO₂NH₂, —SO₂NHR, —SO₂NRR′); sulfones (—SO₂—R); sulfoxides (—SO—R); amines (—NHR, NR′R); ureas (—NH—CO—NH₂, —NH—CO—NHR); ethers (—O—R); halogens; carbamates (—NH—CO—OR); aldehyde-function (—CHO); then also inverse amides; sulfonamides and esters (—NH—CO—R), —NH—SO₂—R, —OOC—R); with halogens; hydroxyl; ethers (—OR); amides (—CONH₂, —CONHR, —CONRR′); sulfonamides (—SO₂NH₂, —SO₂NHR, —SO₂NRR′); amines (—NHR, NR′R); and ureas (—NH—CO—NH₂, —NH—CO—NHR); being especially preferred,

or a pharmaceutically acceptable salt thereof, as such or especially for use in the diagnostic or therapeutic treatment of a warm-blooded animal, especially a human.

Especially preferred is a compound of the formula (I), wherein

A is H; a halo (such as Br); or aryl (such as phenyl or benzyl) or heterocyclyl (such as pyridinyl, indolyl or benzothiophenyl),

wherein the aryl or heterocyclyl may be substituted or unsubstituted with up to 4, preferably up to 2 substituents, wherein the substituents are the same or different and are independently selected from halo (such as Cl or Br); hydroxy; amino; amino lower alkyl (such as dimethylamino); amino lower alkoxy (such as ethoxyamine); lower alkyl (such as methyl); lower alkoxy (such as methoxy); substituted or unsubstituted sulfonamide (such as benzo sulfonamide, chlorobenzene sulfonamide or dichloro benzene sulfonamide); carbamates; R₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl or lower alkyl piperazinyl) where when R4 and R5 together with the N form an heterocyclic ring, said ring may be substituted with 1, 2 or more of any of the substituents described herein, preferably piperazinyl, pyrrolidinyl, alkyl such as methyl, or hydroxy alkyl such as ethanyl. Examples of the heteroring formed by R4 and R5 together with the N include morpholinyl, which can be unsubstituted or substituted with methyl or dimethyl; piperazinyl which can be unsubstituted or substituted with 1, 2 or 3 substituents preferably methyl, oxy or ethanol; or piperadinyl which can be unsubstituted or substituted with 1, 2 or 3 substituents preferably pyrrolidinyl, amine, alkyl amine, methyl amine, dialkyl amine, dimethylamine or diethylamine;

R₂ is H, C₁-C₃ lower alkyl (such as methyl) or aryl (such as phenyl or benzyl) or heterocyclyl (such as pyridyl, indolyl, thiophenyl, thiazolyl or benzothiophenyl), wherein the aryl or heterocyclyl may be substituted or unsubstituted with up to 4, preferably up to 2 substituents, wherein the substituents are the same or different and are independently selected from halo (such as Cl, F or Br), hydroxy; amino; amino lower alkyl; C₁-C₃ lower alkyl; alkoxy (such as methoxy and benzyloxy where the benzyl ring may be substituted or unsubstituted, such as 3,4-dichlorobenzyloxy); sulfoamino; substituted or unsubstituted benzosulfonamide (such as 2,3-dichlorobenzene sulfonamide); substituted or unsubstituted sulfonate (such as chloro-phenyl sulfonate); substituted or unsubstituted ureas (such as 3-trifluoromethyl-phenyl urea or 4-morpholin-4-yl-3-trifluorormethyl-phenyl-urea) or carbamates (such as ethyl-N-phenyl carbamate);

R₃ is H; C₁-C₃ alkyl; phenyl; pyridinyl or oxaz-5-yl;

or a pharmaceutically acceptable salt thereof, as such or especially for use in the diagnostic or therapeutic treatment of a warm-blooded animal, especially a human.

Especially preferred is the use of a compound of formula (I), or a pharmaceutically acceptable salt thereof, in the manufacture of a pharmaceutical preparation for the treatment of an Eph receptor-related (e.g., neurological) injury and disorder. Also preferred is a compound of the formula (I), or a pharmaceutically acceptable salt thereof, as shown above for use in the treatment of an Eph receptor-related (e.g., neurological) injury and disorder.

Pharmaceutical Compositions

The invention relates also to the use of pharmaceutical compositions comprising a compound of formula (I) in the therapeutic (in a broader aspect of the invention also prophylactic) treatment of an Eph receptor-related (e.g., neurological) injury and disorder.

The pharmacologically acceptable compounds of the present invention may be used, for example, for the preparation of pharmaceutical compositions that comprise an effective amount of a compound of the formula (I), or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g. lymphocytes), for the treatment or, in a broader aspect of the invention, prevention of (=prophylaxis against) a disease that responds to inhibition of kinase activity, comprising an amount of a compound of formula (I) or a pharmaceutically acceptable salt thereof, which is effective for said inhibition, especially the in, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of a kinase; which comprises administering an (against the mentioned disease) prophylactically or especially therapeutically effective amount of a compound of formula (I) according to the invention, especially to a warm-blooded animal, for example a human, that, on account of one of the mentioned diseases, requires such treatment.

The dose of a compound of the formula (I) or a pharmaceutically acceptable salt thereof to be administered to warm-blooded animals, for example humans of approximately 70 kg body weight, is preferably from approximately 3 mg to approximately 10 g, more preferably from approximately 10 mg to approximately 1-5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefosse, Paris), “Miglyol 812” (triglyceride of saturated fatty acids with a chain length of C8 to C12, Hüls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

Combinations

The compounds of the invention may also be used to advantage in combination with other agents known to overcome process outgrowth inhibition such as Rho kinase inhibitors; inhibitors of classical PKC isoforms; blocking antibodies against NogoA or the Nogo receptor; Chondroitinase ABC or other reagents that cleave the GAG sidechains off proteoglycans; and agents that increase intrinsic growth capacity of neurons (e.g., cAMP and bcl-2).

By way of a non-exclusive example, the compounds of the invention may be used in combinatorial therapy with an agent capable of blocking myelin inhibitors Nogo, myelin-associated glycoprotein (MAG), or oligodendrocyte-myelin glycoprotein OMgp.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I), can be prepared and administered as described in the art such as in the documents cited above.

The following examples are merely illustrative and not meant to limit the scope of the present claims in any manner.

### EXAMPLES

### Example 1

### EphA4 Mode and Mechanism of Action

In order to distinguish between the forward and bi-directional signaling that ephrins are capable of in the context of axon regeneration, lentiviral expression vectors for wild type and kinase dead EphA4 are generated and overexpressed in purified astrocytes. Cortical neurons are plated on the two astrocytic populations and neurite outgrowth assayed and compared. Biological peptides that have been demonstrated to block the interaction of EphA4 with relevant ligands, consequently inhibiting receptor activation (Murai, K. K., et al., (2003) Mol Cell Neurosci 24(4): p. 1000), are tested for their EphA4 inhibitory activity in the astrocyte/cortical neuron culture system. Identification of the neuronal ligand/ephrin mediating. EphA4 inhibition is achieved by systematically blocking candidate ephrin expression in neurons using RNA interference to knock down ephrins or by using dominant negative ephrin constructs and subsequently plating them on wild type astrocytes. These experiments collectively clarify the mode of EphA4 activation.

To illustrate intracellular events triggered by EphA4 activation, cytokine induced activation of astrocytes are used to explore the precise signaling pathways activated. Cultured astrocytes are treated with inflammatory cytokines (which have been shown to be involved in activating astrocytes) LIF or IFN in the presence or absence of EphA4 blocking peptides, and the cells are lysed and analyzed by Western Blots for the activation of major signaling pathways (MAPK, PI3K, JNK, STAT, RhoA) using appropriate phospho-antibodies. The signaling involved in neurite outgrowth inhibition by EphA4 is assessed by culturing cortical neurons on astrocytes or on CNS myelin or spinal cord extracts in the presence or absence of commercially available pharmacological inhibitors of the major signaling pathways and also the EphA4 inhibitory peptides.

### Example 2

### Autophosphorylation and Ligand-Dependent Phosphorylation Assays

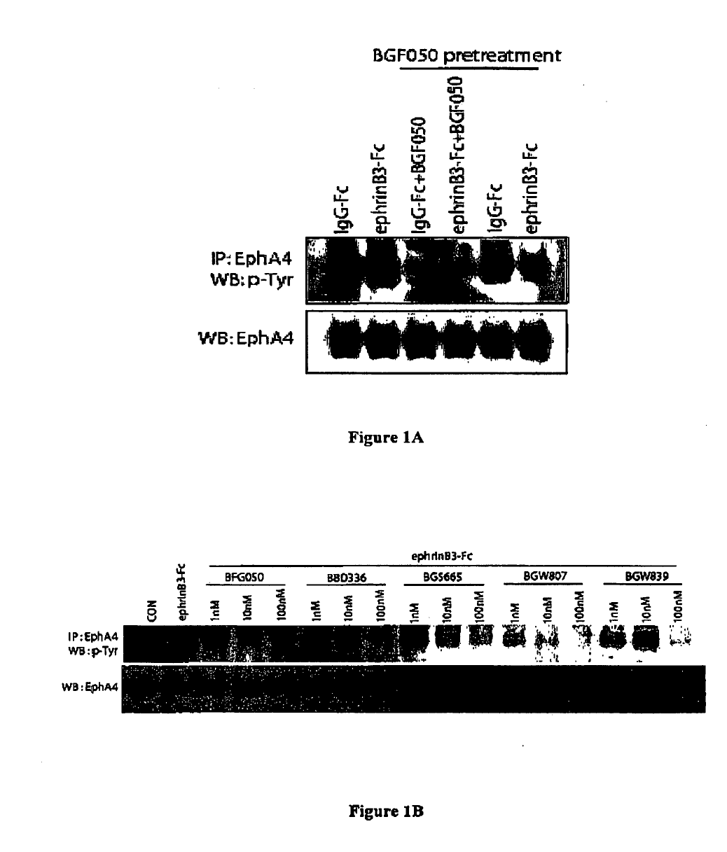

Primary astrocyte cultures are established from neonatal mouse cortex and purified so as to get about 95-98 pure astrocyte cultures. For detecting autophosphorylation the cells are incubated in the presence or absence of pharmacological inhibitors and then directly lysed and subjected to immunoprecipitation and Western analysis (as seen in FIG. 1A). For ligand dependent phosphorylation (as seen in FIG. 1B), the cultures are then serum starved for 36 hours to reduce basal receptor phosphorylation and then stimulated for varying lengths of time with a soluble form of the cognate ligand in the presence or absence of candidate kinase inhibitors or blocking peptides, which are added at various concentrations. Cells are lysed, and the lysates subjected to EphA4 immunoprecipitation and subsequently analysed on Westerns for level of receptor phosphorylation using a phospho-tyrosine antibody.

### Example 3

### In Vitro Assay for Neurite-Outgrowth/Axon Regeneration

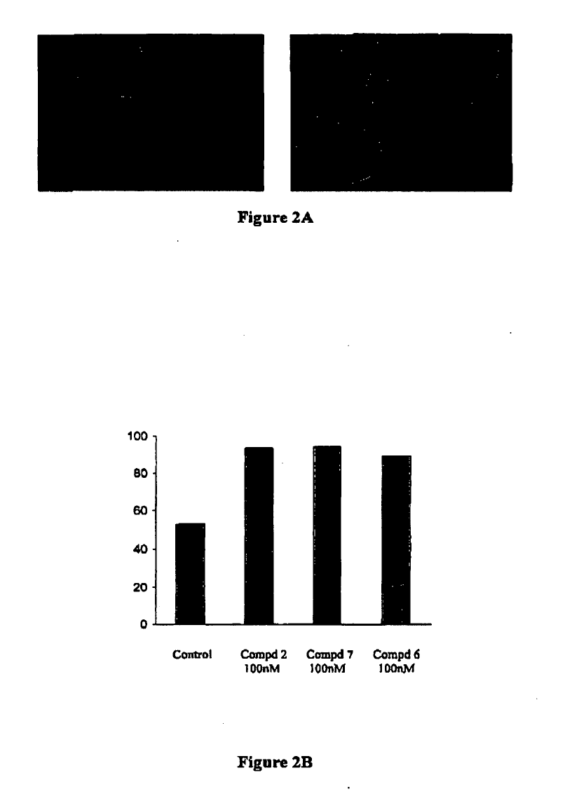

This assay is used to assess neurite outgrowth inhibition of embryonic cortical neurons by Eph receptors expressed on astrocytes or neurite outgrowth inhibition of post-natal cortical neurons by ephrin ligand present in myelin. E16 (embryonic day 16) cortical neurons are plated onto confluent astrocyte monolayers plated in 4-well chamber slides. Pharmacological inhibitors, are added to the medium and the length of longest neurite from each neuron is measured under each condition (FIG. 2A depicts an example of neurons plated in the presence of Compound 1 and visualized with the neuronal marker, Tuj-1) and compared to average neurite length on astrocytes in the absence of any pharmacological agents. FIG. 2B depicts the quantitation of neurite outgrowth effects observed with Compound 6 and Compound 7 (all tested at 100 nM concentration) in cortical cultures plated on astrocytes.

### Example 4

### In Vitro Assay for Astrogliosis—Astrocyte Scratch Wound

Assay Astrocytes are prepared from the cerebral cortex of neonatal C57BL/6 mice (P1-P2). Cells are maintained in Dulbecco's modified Eagle's medium with 10% FBS. 4-7 weeks old astrocytes are plated to confluence in 2 well chamber slides coated with poly-D-lysine for the scratch wound assay and serum starved. 48 hrs after serum starvation, the monolayer of astrocytes is scratched with sterile 200 μl tips and washed twice with PBS to get rid of cell debris. Conditioned medium (+/−cytokines) is added to the wounded astrocytes. The microscopic images of the scratch is captured at a magnification of 10× right after scratch and considered as time point 0.24 hrs, 48 hrs or 72 hrs after scratch, the same region of scratch is imaged and fixed with methanol containing, 1 μg/ml of DAPI to monitor migration and proliferation of astrocytes.

### Example 5

### Proof of Mechanism

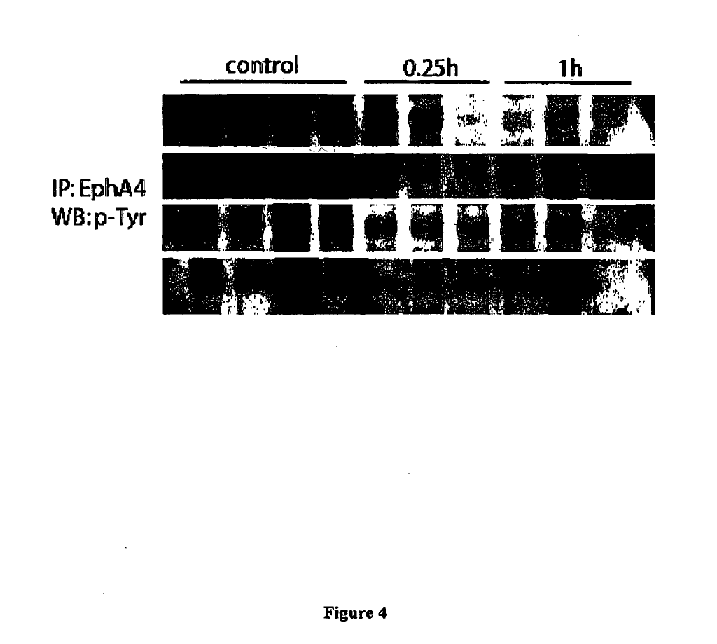

The experiment demonstrates that the compounds of the invention cross the blood brain barrier and effectively block phosphorylation of EphA4 receptor in vivo. Male NMRI mice were injected with relevant compounds at a dose of 10 mg/kg body weight and were sacrificed either 25 minutes or 1 hour following the dosing (0.25 h or 1 h shown in FIG. 4). The brains were removed and one half of each brain was weighed and homogenized in appropriate volume of lysis buffer for 30 seconds (10 seconds pulse and 10 seconds off—3 times). The homogenate was spun at 12,000 g for 30 minutes. Protein amounts were estimated for supernatants (using BCA) and equal amounts of protein for each condition were subjected to EphA4 immunoprecipitation followed by a phospho-tyrosine western blot. Four control animals were used and three experimental animals per time point were used for each of the compounds tested (Compounds 1, 2, 6, and 7)

### Example 6

### High Throughput Screening (HTS)

High throughput screens can be developed to look for selective and specific pharmacological inhibitors of EphA4 activity. Such compounds, as with the compounds of the invention, include kinase inhibitors or binding antagonists that block EphA4 interaction with its ligand and/or specifically block-EphA4 kinase activation. Such compounds, as with the compounds of the invention, can serve as inhibitors that efficiently block EphA4 activity in the context of gliosis and axon regeneration

### Example 7

### In Vivo Target Validation in a Mouse SCI Model

Existing EphA4 inhibitory peptides/hits from the HTS described herein, e.g., the compounds of the invention such as Compounds 1, 6; and 7 can be used for in vivo spinal cord injury (SCI) experiments to determine their efficacy in promoting axon regeneration. Mice are divided into three groups: unlesioned; lesioned with vehicle infusion; and lesioned with drug/peptide infusion. Animals of the lesioned groups undergo spinal hemisection surgery. Drug or vehicle (e.g., containing one of the compounds of the invention) is administered intrathecally via an osmotic pump, and an anterograde tracer is used to track anatomical regeneration of lesioned axons. Appropriate behavioral and electrophysiological assays can be performed to assess functional recovery of sensory and motor functions.

In addition to the SCI model experiments described above, EphA4 inhibitory agents, e.g., the compounds of the invention, can also be tested while Nogo signaling is compromised, to see if this results in a synergistic effect leading to improved functional recovery.

### Examples 8-122

### Syntheses

The following examples employ the abbreviations listed herein, and unless otherwise listed, the following conditions: where no temperatures are given, the reaction takes place at ambient (room) temperature; and ratios of solvents, e.g., in eluents or solvent mixtures, are given in volume by volume (v/v).

Flash chromatography is performed by using silica gel (Merck; 40-63 μm). For thin layer chromatography, pre-coated silica gel (Merck 60 F254) plates are used. Detection of the components is made by UV light (254 nm). HPLC is performed on an Agilent HP 1100 using a Nucleosil 100-3 C₁₈ HD 125×4.0 mm column [1 mL/min.; 20-100% NeCN/0.1% TFA in 7 minutes) (Method A); SpectraSystem SP8800/UV2000 using a Nucleosil 100-5 C₁₈ AB 250×4.6 mm column (2 mL/min.; 2-100% MeCN/0.1% TFA in 10 minutes) (Method B); using a Chromalith Speed ROD RP18 50-4.6 mm column (Merck) (2 mL/min.; 2-160% MeCN/0.1% TFA in 2 minutes) (Method C); or a C8 2.1-50 mm 3 μm column (Waters) (2 mL/min.; 5-95% MeCN/0.1% TFA in 2 minutes) (Method D). ¹H-NMR measurements are performed on a Varian Gemini 400 or a Bruker DRX 500 spectrometer using tetraethylsilane as internal standard. Chemical shifts are expressed in ppm downfield from tetraethylsilane and coupling constants (J) are expressed in Hertz (Hz). Electrospray mass spectra are obtained with a Fisons Instruments VG Platform II. Melting points are measured with a Büch 510 melting point apparatus. Commercially-available solvents and chemicals are used for syntheses.

### Example 8

### 3-{7-Amino-3-[4-(4-methyl-piperazin-1-yl)-phenyl]-pyrazolo[1,5-a]pyrimidin-6-yl}-phenol

6-(3-Benzyloxy-phenyl)-3-[4-(4-methyl-piperazin-1-yl)-phenyl]-pyrazolo[1,5-a]pyrimidin-7-ylamine (Stage 1.1) (25 mg, 0.051 mmol) dissolved in THF (6 mL) is hydrogenated in the presence of Pd/C (10% Engelhard 4505, 6 mg) for 13 hours. After filtration and evaporating the solvent under reduced pressure, the residue is flash chromatographed (silica gel, CH₂Cl₂/MeOH/NH₃=95:5:0.1) to give compound of Example 1 as white solid (14 mg, 0.035 mmol; 70%): ES-MS: M+H=401.1, R_{f} (CH₂Cl₂/MeOH/NH₃=90:10:0.1)=0.33, HPLC: ^{A}t_{Ret}=2.77 minutes.

¹H-NMR (400 MHz, DMSO-d₆): 9.59 (s, 1H, OH), 8.58/8.18 (s/s, 1H/1H, pyrazolopyrimidinyl), 8.01 (d, 9.0 Hz, 2H, phenyl), 7.48 (s, 2H, NH₂), 7.32 (t, 8.5 Hz, 1H, phenyl-OH), 6.99 (d, 9.0 Hz, 2H, phenyl), 6.96 (d, 8.5 Hz, 1H, phenyl-OH), 6.93 (s, 1H, phenyl-OH), 6.80 (d, 8.5 Hz, 1H, phenyl-OH), 3.17/2.48 (m/m, 4H/4H, piperazinyl), 2.24 (s, 3H, CH₃).

### Stage 1.1 6-(3-Benzyloxy-phenyl)-3-[4-(4-methyl-piperazin-1-yl)-phenyl]-pyrazolo[1,5-a]pyrimidin-7-ylamine

4-(4-(4-methyl-piperazin-1-yl)-phenyl)-2H-pyrazol-3-ylamine (Stage 1.2) (100 mg, 0.388 mmol), 2-(3-benzyloxy-phenyl)-3-oxo-propionitrile (Stage 1.3) (98 mg, 0.388 mmol), HCl (2.5 mM in EtOH; 1.55 mmol, 0.9 mL) dissolved in EtOH (1 mL) are stirred for 17 hours at RT. After adding H₂O (4 mL) and K₂CO₃ (250 mg), the reaction mixture is extracted with CH₂Cl₂ (20 mL, 2×). The combined organic phases are washed with H₂O (10 mL), dried (Na₂SO₄), concentrated under reduced pressure and flash chromatographed (silica gel, 2.5×15 cm, CH₂Cl₂/MeOH=9:1) to give compound of Stage 1.1 as white solid (60 mg, 0.122 mmol; 32%); ES-MS: M+H=491.0, R_{f} (CH₂Cl₂/MeOH/NH₃=90:10:0.1)=0.42; HPLC: ^{A}t_{Ret}=4.69 minutes.

¹H-NMR (400 MHz, DMSO-d₆): 8.79/8.21 (s/s, 1H/1H, pyrazolopyrimidinyl), 8.03 (d, 9.0 Hz, 2H, phenyl), 7.53 (s, 2H, NH₂), 7.44 (m, 5H, benzyl), 7.32 (t, 8.5 Hz, 1H, phenyl-OH), 7.29 (s, 1H, phenyl-OH), 7.13 (d, 8.5 Hz, 1H, phenyl-OH), 7.06 (d, 8.5 Hz, 1H, phenyl-OH), 6.97 (d, 9.0 Hz, 2H, phenyl), 5.19 (s, 2H, benzyl), 3.17/2.48 (m/m, 4H/4H, piperazinyl), 2.24 (s, 3H, CH₃).

### Stage 1.2 4-(4-(4-Methyl-piperazin-1-yl)-phenyl)-2H-pyrazol-3-ylamine

2-[4-(4-Methyl-piperazin-1-yl)-phenyl]-3-oxo-propionitrile (Stage 1.4) (370 mg, 1.52 mmol), hydrazine monohydrate (0.185 mL, 3.8 mmol) dissolved in AcOH are stirred at 98° C. for 3 hours. After cooling down to RT, H₂O (8 mL) and concentrated HCl (0.8 mL) are added and the reaction mixture is stirred under reflux for 20 minutes. After cooling down to RT, the reaction mixture is adjusted to alkaline pH by slowly adding NH₃ (255%). Precipitating material is filtered-off and kept for further purification. The reaction solution is extracted with CH₂Cl₂ (50 mL, 3×), dried (Na₂SO₄) and concentrated under reduced pressure. Precipitated and extracted material is combined and flash chromatographed (silica gel, 3.0×18 cm, CH₂Cl₂/MeOH/NH₃=9:1:01) to give compound of Stage 1.2 as white solid (277 mg, 1.08 mmol; 71%), ES-MS: M+H=258.1, R_{f} (CH₂C₂/MeOH/NH₃=90:10:0.1)=0.28; HPLC: ^{A}t_{Ret}=4.33 minutes.

¹H-NMR (400 MHz, DMSO-d₆): 11.55 (s/broad, 1H, NH), 7.55 (s, 1H, pyrolyl), 7.35 (d, 9.0 Hz, 2H, phenyl), 6.91 (d, 9.0 Hz, 2H, phenyl), 4.55 (s/broad, 2H, NH₂), 3.10/2.46 (m/m, 4H/4H, piperazinyl), 2-23 (s, 3H, CH₃).

### Stage 1.3 2-(3-Benzyloxy-phenyl)-3-oxo-propionitrile

Na (260 mg, 11.3 mmol) is dissolved in absolute EtOH (11 mL) under Ar during 20 minutes. After adding (3-benzylox-phenyl)-acetonitrile (1.9 g, 8.68 mmol) and ethyl formate (1.05 mL, 13.0 mmol), the reaction mixture is stirred under reflux for 2 hours. After evaporating the solvent under reduced pressure, adding H₂O (20 mL), and adjusting to pH=4.0 by adding AcOH, the reaction suspension is extracted with CH₂Cl₂ (30 mL, 2×). The combined organic phases are washed with H₂O (10 mL), dried (Na₂SO₄), concentrated under reduced pressure and flash chromatographed (silica gel, 4.5×25 cm, CH₂Cl₂/MeOH=98:2) to give compound of Stage 1.3 as white solid (780 mg, 3.11 mmol; 36%); ES-MS: M−H=250.0, R_{f} (CH₂Cl₂/MeOH=95:5)=0.49; HPLC: ^{A}t_{Ret}=6.07 minutes.

¹H-NMR (400 MHz, DMSO-d₆): 7.45-7.25/6.98-6.88 (m/m, 8H, aryl), 5.09 (s, 2H, CH₂), 3.98 (s, 2H, CH₂).

### Stage 1.4 2-[4-(4-Methyl-piperazin-1-yl)-phenyl]-3-oxo-propionitrile

Na 160 mg (7.0 mmol) is dissolved in absolute EtOH (6 mL) under Ar during 10 minutes. After adding [4-(4-methyl-piperazin-1-yl)-phenyl]-acetonitrile (Stage 1.5) (1 g, 4.64 mmol) and ethyl formate (0.56 mL, 7.0 mmol), the reaction mixture is stirred under reflux for 1 hour. After washing the reaction pulp with ether (50 mL, 3×), the solid residue is dissolved in H₂O (60 mL) and adjusted to pH=3.9 by adding AcOH. The aqueous solution is extracted with CH₂Cl₂ (50 mL, 3×). The combined organic phases are washed with H₂O (50 mL). Both aqueous phases are combined and lyophilized. The resulting residue is crystallized from MeOH/CH₂Cl₂ to give compound of Stage 1.4 as white crystals (721 mg, 3.0 mmol; 64%); ES-MS: M+H=244.1; HPLC: ^{A}t_{Ret}=2.43 minutes.

¹H-NMR (400 MHz, DMSO-d₆): the compound forms a tautomeric equilibrium in solution: 7.87/7.77 (s/s, 1H, CH═/CH—OH), 7.53/7.17 (d/d, 9.0 Hz, 2H, phenyl), 7.84/7.822 (d/d, 9.0 Hz, 2H, phenyl), 3.10 (m, 4H, piperazinyl), 2.57/2.51 (m/m, 4H, piperazinyl), 2.29/2.26 (s, 3H, CH₃).

### Stage 1.5 [4-(4-Methyl-piperazin-1-yl)-phenyl]-acetonitrile

(4-Bromo-phenyl)-acetonitrile (5 g, 25.5 mmol), 1-methyl-piperazine (3.4 mL, 30.6 mmol), K₂CO₃ (7.68 g, 35.7 mmol), Pd(AcO)₂ (280 mg-1.275 mmol), 2-(di-tert-butylphosphino)-biphenyl (1.14 g, 3.825 mmol) dissolved in 1,2-dimethoxyethane (70 mL) are stirred under Ar at 85° C. for 20 hours. After adding H₂O (100 mL), the reaction mixture is extracted with CH₂Cl₂ (100 mL, 3×). The combined organic phases are washed with H₂O (100 mL), dried Na₂SO₄), concentrated under reduced pressure and flash chromatographed (silica gel, 4.5×34 cm, CH₂Cl₂/MeOH=95:5) to give compound of Stage 1.5 as white solid (2.8 g, 13 mmol; 51%); ES-MS: M+H=216.1; R_{f} (CH₂Cl₂/MeOH=9:1)=0.47; HPLC: ^{A}t_{Ret}=2.24 minutes.

¹H-NMR (400 MHz, DMSO-d₆): 7.14/6.91 (d/d, 9.5 Hz, 2H/2H, phenyl), 7.53 (s, 2H, NH₂), 7.44 (m, 5H, benzyl), 7.32 (t, 8.5 Hz, 1H, phenyl-OH), 7.29 (s, 1H, phenyl-OH), 3.84 (s, 2H, benzyl), 3.09/2.42 (t/t, 5.0 Hz, 4H/4H, piperazinyl), 2.18 (s, 3H, CH₃).

### Example 9

### 6-(3-Methoxy-phenyl)-3-[4-(4-methyl-piperazin-1-yl)-phenyl]-pyrazolo[1,5-a]pyrimidin-7-ylamine

6-(3-Methoxy-phenyl)-3-[4-(4-methyl-piperazin-1-yl)-phenyl]-pyrazolo[1,5-a]pyrimidin-7-ylamine is synthesized by condensation of compound of Stage 1.2 and 2-(3-methoxy-phenyl)-3-oxo-propionitrile (Stage 2.1) analogously to the preparation of compound of Example 1. Yield: 48%, solid powder; ES-MS: M+H=415.1; HPLC: ^{A}t_{Ret}=3.45 minutes.

¹H-NMR (400 MHz, DMSO-d₆): 8.59/8.23 (s/s, 1H/1H, pyrazolopyrimidinyl), 8.06 (d, 9.0 Hz, 2H, phenyl), 7.55 (s, 2H, NH₂), 7.43 (t, 8.5 Hz, 1H, phenyl-OMe), 7.10 (d, 8.5 Hz, 1H, phenyl-OMe), 7.08 (s, 1H, phenyl-OMe), 6.80 (d, 8.5 Hz, 1H, phenyl-OMe), 6.98 (d, 9.0 Hz, 2H, phenyl), 3.83 (s, 3H, CH₃—O), 3.16/2.47 (m/m, 4H/4H, piperazinyl), 2.25 (s, 3H₁CH₃).

### Stage 2.1 2-(3-Methoxy-phenyl)-3-oxo-propionitrile

Z-(3-Methoxy-phenyl)-3-oxo-propionitrile is synthesized analogously to the preparation of compound of Stage 1.3: Yield: 76%; white powder; ES-MS: M−H=174.0; HPLC: ^{A}t_{Ret}=4.75 minutes.

¹H-NMR (400 MHz, DMSO-d₆): the compound forms a tautomeric equilibrium in solution: 8.09/7.67 (s/s, 1H, CH═/CH—OH), 7.38-7.23 (m, 2H, phenyl), 7.01-6.97 (m, 1H, phenyl), 6.88-6.79 (m, 1H, phenyl), 3.74 (s/broad, 3H, CH₃—O).

### Example 10

### 6-(3,5-Dimethoxy-phenyl)-3-[4-(4-methyl-piperazin-1-yl)-phenyl]-pyrazolo[1,5-a]pyrimidin-7-ylamine

6-(3,5-Dimethoxy-phenyl)-3-[4-(4-methyl-piperazin-1-yl)-phenyl]-pyrazolo[1,5-a]pyrimidin-7-ylamine is synthesized by condensation of compound of Stage 1.2 and 2-(3,5-dimethoxy-phenyl)-3-oxo-propionitrile (Stage 3.1) analogously to the preparation of compound of Example 1. Yield: 44%, solid powder; ES-MS: M+H=445.0; HPLC: ^{A}t_{Ret}=3.77 minutes.

¹H-NMR (400 MHz, DMSO-d₆): 8.59/8.23 (s/s, 1H/1H, pyrazolopyrimidinyl), 8.06 (d, 9.0 Hz, 2H, phenyl), 7.55 (s, 2H, NH₂), 7.43 (t, 8.5 Hz, 1H, phenyl-OMe), 7.10 (d, 8.4 Hz, 1H, phenyl-OMe), 7.57 (s; 2H, NH₂), 7.01 (d, 9.0 Hz, 2H, phenyl), 6.89 (s, 2H, phenyl-OMe), 6.54 (s, 1H, phenyl-OMe), 3.83 (s, 6H, CH₃—O), 3.16/2.47 (m/m, 4H/4H, piperazinyl), 2.24 (s, 3H, N—CH₃).

### Stage 3.1 2-(3,5-Dimethoxy-phenyl)-3-oxo-propionitrile

2-(3,5-Dimethoxy-phenyl)-3-oxo-propionitrile is synthesized analogously to the preparation of compound of Stage 1.3. Yield: 48%; white powder; ES-MS: M+H=206.0; HPLC: ^{A}t_{Ret}=4.79 minutes.

¹H-NMR (400 MHz, DMSO-d₆): the compound forms a tautomeric equilibrium in solution: 8.11/7.68 (s/s, 1H, CH═/CH—OH), 6.85/6.54 (s/s, 2H, phenyl), 6.44/6.38 (s/s, 1H, phenyl), 3.74 (s/broad, 6H, CH₃—O).

### Example 11

### Stage 1.1: 6-(3-Benzyloxy-phenyl)-3-[4-(4-methyl-piperazin-1-yl)-phenyl]-pyrazolo[1,5-a]pyrimidin-7-ylamine

Prepared by the step disclosed in Stage 1.1

### Examples 12-76

The following Examples enlisted on Table 1 are synthesized analogously to the preparation of Example 8. As far as not being commercially-available, the syntheses of intermediates for the preparation of compounds of Examples 12-76 are described below Table II. In cases where the title compounds carry a free amino group (Examples 59-61), the final products are generated from their corresponding nitro-function carrying precursors by hydrogenation in the presence of Pd/C (10%) in THF/MeOH during several hours.

TABLE II

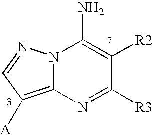

| Nb. | A | R2 | R3 | Analytical Data |
|---|---|---|---|---|
| 12 | 4-(4-Methyl-piperazin-1- | 4-Chlorophenyl | H | ES-MS |
| | yl)phenyl | | | [M + 1]⁺ = |
| | | | | 419.0/421.0; |
| | | | | HPLC ^{A}t_{Ret} = |
| | | | | 3.71 minutes |
| 13 | 4-(4-Methyl-piperazin-1- | 3-Chlorophenyl | H | ES-MS |
| | yl)phenyl | | | [M + 1]⁺ = |
| | | | | 419.0/421.0; |
| | | | | HPLC ^{A}t_{Ret} = |
| | | | | 3.92 minutes |
| 14 | 4-(4-Methyl-piperazin-1- | Phenyl | H | ES-MS |
| | yl)phenyl | | | [M + 1]⁺ = |
| | | | | 385.1; |
| | | | | PLC ^{A}t_{Ret} = |
| | | | | 3.31 minutes |
| 15 | 4-(4-Methyl-piperazin-1- | Phenyl | Methyl | ES-MS |
| | yl)phenyl | | | [M + 1]⁺ = |
| | | | | 399.1; |
| | | | | HPLC ^{A}t_{Ret} = |
| | | | | 3.34 minutes |
| 16 | 4-(4-Methyl-piperazin-1- | Methyl | Phenyl | ES-MS |
| | yl)phenyl | | | [M + 1]⁺ = |
| | | | | 399.0; |
| | | | | HPLC ^{A}t_{Ret} = |
| | | | | 3.36 minutes |
| 17 | 4-dimethyl amino phenyl | 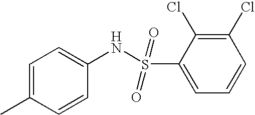 | H | m.p. 143-146° C.;R_{f}(CH₂Cl₂/MeOH =98:2): 0.32ES-MS[M + 1]⁺ =552.8;HPLC ^{A}t_{Ret} =4.72 minutes |
| 18 | 4-dimethyl amino phenyl | 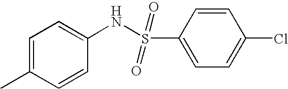 | H | m.p. 186-1.88° C.;R_{f}(CH₂Cl₂/MeOH =98:2): 0.50;ES-MS[M + 1]⁺ =519.8;HPLC ^{A}t_{Ret} =5.27 minutes |
| 19 | Phenyl | 4-Methoxyphenyl | Methyl | ES-MS |
| | | | | [M + 1]⁺ = |
| | | | | 331.1; |
| | | | | HPLC ^{B}t_{Ret} = |
| | | | | 6.4 minutes |
| 20 | 4-Methoxy-phenyl | Phenyl | Methyl | ES-MS |
| | | | | [M + 1]⁺ = |
| | | | | 331.1; |
| | | | | HPLC ^{B}t_{Ret} = |
| | | | | 6.3 minutes |
| 21 | 4-Methoxy-phenyl | 4-Bromophenyl | Methyl | ES-MS |
| | | | | [M + 1]⁺ = |
| | | | | 408.9/410.9; |
| | | | | HPLC ^{B}t_{Ret} = |
| | | | | 7.1 minutes |
| 22 | Phenyl | 4-Bromophenyl | Methyl | ES-MS |
| | | | | [M + 1]⁺ = |
| | | | | 378.9/380.9; |
| | | | | HPLC ^{B}t_{Ret} = |
| | | | | 7.0 minutes |
| 23 | Phenyl | 2,6-Dichlorophenyl | H | ES-MS |
| | | | | [M + 1]⁺ = |
| | | | | 354.9/356.9; |
| | | | | HPLC ^{B}t_{Ret} = |
| | | | | 7.9 minutes |
| 24 | 3-Methoxy-phenyl | Phenyl | H | ES-MS |
| | | | | [M + 1]⁺ = |
| | | | | 317.1; |
| | | | | HPLC ^{B}t_{Ret} = |
| | | | | 6.8 minutes |
| 25 | Br | H | Phenyl | ES-MS |
| | | | | [M + 1]⁺ = |
| | | | | 288.9/290.9; |
| | | | | HPLC ^{B}t_{Ret} = |
| | | | | 6.3 minutes |
| 26 | 4-(4-Methyl-piperazin-1-yl)phenyl | 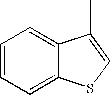 | H | ES-MS[M + 1]⁺ =441.0;HPLC ^{A}t_{Ret} =1.91 minutes |
| 27 | 4-Bromo-phenyl | H | Phenyl | ES-MS |
| | | | | [M + 1]⁺ = |
| | | | | 367.0; |
| | | | | HPLC ^{A}t_{Ret} = |
| | | | | 2.56 minutes |
| 28 | 4-(4-Methyl-piperazin-1-yl)phenyl | 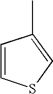 | H | ES-MS[M + 1]⁺ =391.1;HPLC ^{A}t_{Ret} =1.49 minutes |
| 29 | 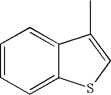 | 3-Methoxyphenyl | H | ES-MS[M + 1]⁺ =373.2;HPLC ^{C}t_{Ret} =2.22 minutes |
| 30 | 4-(4-Methyl-piperazin-1- | Benzyl | H | ES-MS |
| | yl)phenyl | | | [M + 1]⁺ = |
| | | | | 399.2; |
| | | | | HPLC ^{C}t_{Ret} = |
| | | | | 1.79 minutes |
| 31 | 3-(4-Methyl-piperazin-1- | 3-Methoxyphenyl | H | ES-MS |
| | yl)phenyl | | | [M + 1]⁺ = |
| | | | | 415.2; |
| | | | | HPLC ^{C}t_{Ret} = |
| | | | | 1.82 minutes |
| 32 | 4-(4-Methyl-piperazin-1-yl)phenyl | 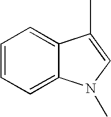 | H | ES-MS[M + 1]⁺ =438.2;HPLC ^{C}t_{Ret} =1.91 minutes |
| 33 | 4-(4-Methyl-piperazin-1- | 4-Methoxyphenyl | H | ES-MS |
| | yl)phenyl | | | [M + 1]⁺ = |
| | | | | 415.2; |
| | | | | HPLC ^{C}t_{Ret} = |
| | | | | 2.04 minutes |
| 34 | 4-(4-Methyl-piperazin-1- | 2-Methoxyphenyl | H | ES-MS |
| | yl)phenyl | | | [M + 1]⁺ = |
| | | | | 415.2; |
| | | | | HPLC ^{C}t_{Ret} = |
| | | | | 1.75 minutes |
| 35 | Pyridin-3-yl | 3-Methoxyphenyl | H | ES-MS |
| | | | | [M + 1]⁺ = |
| | | | | 318.6; |
| | | | | HPLC ^{C}t_{Ret} = |
| | | | | 1.84 minutes |
| 36 | 3-(4-Methyl-piperazin-1- | 3-Hydroxyphenyl | H | ES-MS |
| | yl)phenyl | | | [M + 1]⁺ = |
| | | | | 401.6; |
| | | | | HPLC ^{A}t_{Ret} = |
| | | | | 1.78 minutes |
| 37 | 2-Methoxy-5-(4-Methyl- | 3-Benzyloxyphenyl | H | ES-MS |
| | piperaizn-1-yl)phenyl | | | [M + 1]⁺ = |
| | | | | 521.3; |
| | | | | HPLC ^{C}t_{Ret} = |
| | | | | 2.07 minutes |
| 38 | 2-Methoxy-5-(4-Methyl- | 3-Hydroxyphenyl | H | ES-MS |
| | piperazin-1-yl)phenyl | | | [M + 1]⁺ = |
| | | | | 431.7; |
| | | | | HPLC ^{C}t_{Ret} = |
| | | | | 1.66 minutes |
| 39 | 4-(4-Methyl-piperazin-1- | 2-Benzyloxyphenyl | H | ES-MS |
| | yl)phenyl | | | [M + 1]⁺ = |
| | | | | 491.2; |
| | | | | HPLC ^{C}t_{Ret} = |
| | | | | 1.77 minutes |
| 40 | 4-(4-Methyl-piperazin-1- | 2-Hydroxyphenyl | H | ES-MS |
| | yl)phenyl | | | [M + 1]⁺ = |
| | | | | 401.2; |
| | | | | HPLC ^{D}t_{Ret} = |
| | | | | 1.37 minutes |
| 41 | 4-(4-Methyl-piperazin-1- | 4-Benzyloxyphenyl | H | ES-MS |
| | yl)phenyl | | | [M + 1]⁺ = |
| | | | | 491.2; |
| | | | | HPLC ^{C}t_{Ret} = |
| | | | | 1.85 minutes |
| 42 | 4-(4-Methyl-piperazin-1- | 4-Hydroxyphenyl | H | ES-MS |
| | yl)phenyl | | | [M + 1]⁺ = |
| | | | | 401.2; |
| | | | | HPLC ^{D}t_{Ret} = |
| | | | | 1.32 minutes |
| 43 | 3-(4-Methyl-piperazin-1- | 2-Benzyloxyphenyl | H | ES-MS |
| | yl)phenyl | | | [M + 1]⁺ = |
| | | | | 491.3; |
| | | | | HPLC ^{A}t_{Ret} = |
| | | | | 2.02 minutes |
| 44 | 3-(4-Methyl-piperzazin-1- | 2-Hydroxyphenyl | H | ES-MS |
| | yl)phenyl | | | [M + 1]⁺ = |
| | | | | 401.3; |
| | | | | HPLC ^{A}t_{Ret} = |
| | | | | 1.71 minutes |
| 45 | 3-(4-Methyl-piperazin-1- | 4-Benzyloxyphenyl | H | ES-MS |
| | yl)phenyl | | | [M + 1]⁺ = |
| | | | | 491.3; |
| | | | | HPLC ^{C}t_{Ret} = |
| | | | | 2.05 minutes |
| 46 | 3-(4-Methyl-piperazin-1- | 4-Hydroxyphenyl | H | ES-MS |
| | yl)phenyl | | | [M + 1]⁺ = |
| | | | | 401.3; |
| | | | | HPLC ^{C}t_{Ret} = |
| | | | | 1.70 minutes |
| 47 | 2-Methoxy-5-(4-Methyl- | 2-Benzyloxyphenyl | H | ES-MS |
| | piperazin-1-yl)phenyl | | | [M + 1]⁺ = |
| | | | | 521.3; |
| | | | | HPLC ^{C}t_{Ret} = |
| | | | | 1.99 minutes |
| 48 | 2-Methoxy-5-(4-Methyl- | 2-Hydroxyphenyl | H | ES-MS |
| | piperazin-1-yl)phenyl | | | [M + 1]⁺ = |
| | | | | 431.3; |
| | | | | HPLC ^{C}t_{Ret} = |
| | | | | 1.70 minutes |
| 49 | 2-Methoxy-5-(4-Methyl- | 4-Benzyloxyphenyl | H | ES-MS |
| | piperazin-1-yl)phenyl | | | [M + 1]⁺ = |
| | | | | 521.3; |
| | | | | HPLC ^{C}t_{Ret} = |
| | | | | 2.05 minutes |
| 50 | 2-Methoxy-5-(4-Methyl- | 4-Hydroxyphenyl | H | ES-MS |
| | piperazin-1-yl)phenyl | | | [M + 1]⁺ = |
| | | | | 431.3; |
| | | | | HPLC ^{C}t_{Ret} = |
| | | | | 1.68 minutes |
| 51 | 1-Methyl-1H-indol-3-yl | 3-Benzyloxyphenyl | H | ES-MS |
| | | | | [M + 1]⁺ = |
| | | | | 446.2; |
| | | | | HPLC ^{C}t_{Ret} = |
| | | | | 2.39 minutes |
| 52 | 1-methyl-1H-indol-3-yl | 3-Hydroxyphenyl | H | ES-MS |
| | | | | [M + 1]⁺ = |
| | | | | 356.6; |
| | | | | HPLC ^{C}t_{Ret} = |
| | | | | 2.06 minutes |
| 53 | 3-Pyridyl | 3-Hydroxyphenyl | H | ES-MS |
| | | | | [M + 1]⁺ = |
| | | | | 304.1; |
| | | | | HPLC ^{C}t_{Ret} = |
| | | | | 1.72 minutes |
| 54 | 2-methoxy phenyl | 3-Benzyloxyphenyl | H | ES-MS |
| | | | | [M + 1]⁺ = |
| | | | | 423.2; |
| | | | | HPLC ^{C}t_{Ret} = |
| | | | | 2.10 minutes |
| 55 | 2-methoxy phenyl | 3-Hydroxyphenyl | H | ES-MS |
| | | | | [M + 1]⁺ = |
| | | | | 333.2; |
| | | | | HPLC ^{C}t_{Ret} = |
| | | | | 1.98 minutes |
| 56 | 3-(4-Methyl-piperazin-1-yl)phenyl | 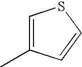 | H | ES-MS[M + 1]⁺ =391.1;HPLC ^{D}t_{Ret} =1.56 minutes |
| 57 | 2-Methoxy-5-(4-Methyl-piperazin-1-yl)phenyl | 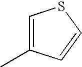 | H | ES-MS[M + 1]⁺ =421.1;HPLC ^{D}t_{Ret} =1.49 minutes |
| 58 | 4-(4-Methyl-piperazin-1-yl)phenyl | 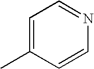 | H | ES-MS[M + 1]⁺ =386.2;HPLC ^{C}t_{Ret} =0.44 minutes |
| 59 | 3-(4-Methyl-piperazin-1-yl)phenyl | 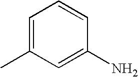 | H | ES-MS[M + 1]⁺ =400.2;HPLC ^{C}t_{Ret} =1.57 minutes |
| 60 | 4-(4-Methyl-piperazin-1-yl)phenyl | 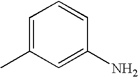 | H | ES-MS[M + 1]⁺ =400.0;HPLC ^{D}t_{Ret} =1.75 minutes |
| 61 | 4-(4-Methyl-piperazin-1-yl)phenyl | 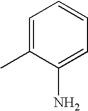 | H | ES-MS[M + 1]⁺ =400.2;HPLC ^{D}t_{Ret} =1.40 minutes |
| 62 | 4-(4-Methyl-piperazin-1- | 4-methyl thiazol-2-yl | H | ES-MS |
| | yl)phenyl | | | [M + 1]⁺ = |
| | | | | 405.6; |
| | | | | HPLC ^{C}t_{Ret} = |
| | | | | 2.11 minutes |
| 63 | 2-Methoxy-5-(4-Methyl-piperazin-1-yl)phenyl | 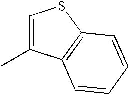 | H | ES-MS[M + 1]⁺ =471.5;HPLC ^{C}t_{Ret} =1.80 minutes |
| 64 | 4-methoxy phenyl | 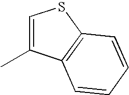 | H | ES-MS[M + 1]⁺ =373.7;HPLC ^{A}t_{Ret} =2.24 minutes |
| 65 | 3-methoxy phenyl | 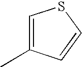 | H | ES-MS[M + 1]⁺ =323.1;HPLC ^{C}t_{Ret} =2.09 minutes |
| 66 | 3-methoxy phenyl | 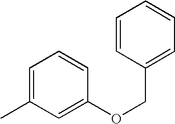 | H | ES-MS[M + 1]⁺ =423.2;HPLC ^{C}t_{Ret} =2.38 minutes |
| 67 | 3-methoxy phenyl | 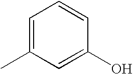 | H | ES-MS[M + 1]⁺ =333.6;HPLC ^{C}t_{Ret} =2.02 minutes |
| 68 | 4-(4-Methyl-piperazin-1-yl)phenyl | 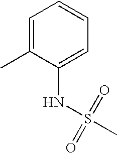 | H | ES-MS[M + 1]⁺ =448.2;HPLC: ^{C}t_{Ret} =1.62 minutes |
| 69 | 4-(4-Methyl-piperazin-1-yl)phenyl | 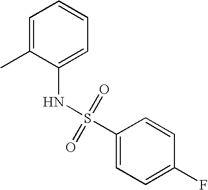 | H | ES-MS[M + 1]⁺ =558.2;HPLC: ^{C}t_{Ref} =1.87 minutes |
| 70 | 4-(4-Methyl-piperazin-1-yl)phenyl | 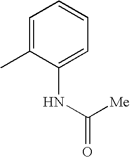 | H | ES-MS[M + 1]⁺ =442.1;HPLC: ^{D}t_{Ret} =1.39 minutes |
| 71 | 3-(4-Methyl-piperazin-1-yl)phenyl | 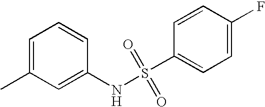 | H | ES-MS[M + 1]⁺ =558.4;HPLC: ^{C}t_{Ret} =2.00 minutes |
| 72 | 3-(4-Methyl-piperazin-1-yl)phenyl | 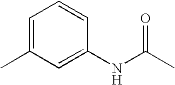 | H | ES-MS[M + 1]⁺ =442.6;HPLC: ^{C}t_{Ret} =1.70 minutes |
| 73 | 4-(4-Methyl-piperazin-1-yl)phenyl | 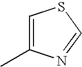 | H | ES-MS[M + 1]⁺ =391.5;HPLC: ^{C}t_{Ret} =1.79 minutes |
| 74 | 4-(4-Methyl-piperazin-1-yl)phenyl | 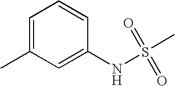 | H | ES-MS[M + 1]⁺ =47824;HPLC: ^{C}t_{Ret}:1.76 minutes |
| 75 | 4-(4-Methyl-piperazin-1-yl)phenyl | 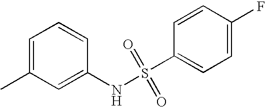 | H | ES-MS[M + 1]⁺ =558.2;HPLC: ^{D}t_{Ret}:1.94 minutes |
| 76 | 4-(4-Methyl-piperazin-1-yl)phenyl | 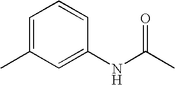 | H | ES-MS[M + 1]⁺ =442.2;HPLC: ^{C}t_{Ret}:1.62 minutes |

### Stage 5.1: 2-(4-Chloro-phenyl)-3-oxo-propionitrile

2-(4-Chloro-phenyl)-3-oxo-propionitrile is prepared analogously to the preparation of compound of Stage 1.3: 89%, ES-MS [M−1]⁻=177.9/179.9; HPLC ^{A}t_{Ret}−5.67 minutes.

### Stage 6.1: 2-(3-Chloro-phenyl)-3-oxo-propionitrile

2-(3-Chloro-phenyl)-3-oxo-propionitrile is prepared analogously to the preparation of compound of Stage 1.3: 89%; ES-MS [M−1]⁻=177.9/179.9; HPLC ^{A}t_{Ret}=5.60 minutes.

### Stage 8.1 3-Oxo-2-phenyl-butyronitrile

3-Oxo-2-phenyl-butyronitrile is prepared analogously to the preparation of compound of Stage 1.3: 62%, white crystals, m.p. >215° C.; ES-ES-MS M−H=157.9, R_{f} (hexane/AcOEt=1:1)=0.57.

¹H-NMR (400 MHz, DMSO-d₆): 7.84 (d, 9.0 Hz, 2H), 7.04 (t, 9.0 Hz, 2H), 6.68 (t, 9.0 Hz, 1H), 3.21 (s/broad, 1H, CH), 2.03 (s, 3H, CH₃).

### Stage 9.1 2-Methyl-3-oxo-3-phenyl-propionitrile

2-Methyl-3-oxo-3-phenyl-propionitrile is prepared analogously to the procedure of Yoo et al., Tetrahedron Lett., Vol. 43, No. 27, pp. 4813-4815 (2002). 2-Bromo-propionitrile (0.965 mL, 11.05 mmol) and In-powder (975 mg, 8.5 mmol) are stirred under Ar in THF (15 mL) for 1 hour. After adding benzoylnitrile (735 mg, 5.6 mmol) during 2 minutes, the reaction mixture is stirred at 60° C. in a microwave oven (Emrys optimizer, personal chemistry, Sweden) for 30 minutes. After filtration over Hyflo and washing with THF (5 mL), the reaction solution is concentrated under reduced pressure and partitioned between ether (150 mL) and phosphate buffer (pH=7, 150 mL). After separation of the organic phase, the aqueous phase is extracted with ether (150 mL). The combined organic phases are washed with brine 30 mL), dried Na₂SO₄), concentrated under reduced pressure and flash chromatography (silica gel, 2×18 cm, hexane/AcOEt=3:1) to compound of Stage 9.1 as slightly yellowish oil (300 mg, 1.9 mmol; 34%); ES-MS: M−H=157.9; R_{f} (hexane/AcOEt=1:1)=0.60.

¹H-NMR (400 MHz, DMSO-d): 8.06 (d, 8.5 Hz, 2H), 7.74 (t, 8.5 Hz, 1H), 7.62 (t, 8.5 Hz, 2H), 5.17 (q, 8.5 Hz, 1H, CH), 1.52 (s, 3H, C₁H₃).

The compound of Example 24 is synthesized analogously to the preparation of compound of Stage 1.1 by condensing 2,3-dichloro-N-[4-(cyano-formyl-methyl)-phenyl]-benzenesulfonamide (Stage 10.1) and 4-(4-dimethylamino-phenyl)-2H-pyrazol-3-ylamine (Stage 10.3).

### Stage 10.1 2,3-Dichloro-N-[4-(cyano-formyl-methyl)-phenyl]-benzenesulfonamide

Under an atmosphere of N₂ is added portion-wise freshly-cut pieces of sodium (2.3 g total, 100 mmol) to EtOH abs. (230 mL) within 15 minutes which is a slightly exothermic (up to 43° C.). After all sodium is dissolved (ca. 1 hour) 2,3-dichloro-N-(4-cyanomethyl-phenyl)-benzene-sulfonamide (Stage 10.2) (26.27 g, 77 mmol) and formic acid ethyl ester (11.2 mL, 139 mmol) is added to the colorless solution at RT. The mixture is heated to reflux for 2 hours. After cooling to RT, the solvent is removed under reduced pressure and the residue dissolved in H₂O (20 mL), followed by addition of AcOH (200 mL; pH 4). The aqueous layer is extracted with CH₂Cl₂ (2×, 500 mL), the combined organics are washed with H₂O and dried over Na₂SO₄. Purification is done by repeated chromatography (silica gel, EtOAc and CH₂Cl₂/MeOH=98:2) to obtain 2,3-dichloro-N-[4-(cyano-formyl-methyl)-phenyl]-benzenesulfonamide (233 mg, 1% yield) as beige crystals: m.p. 88-102° C.; (CH₂Cl₂/MeOH=95:5): 0.22; ES-MS [M+1]⁺=368; HPLC ^{B}t_{Ret}=5.61 minutes.

### Stage 10.2 2,3-Dichloro-N-(4-cyanomethyl-phenyl)-benzenesulfonamide

To the solution of 4-aminobenzylcyanide (12 g, 90.8 mmol) in pyridine (11 mL) at RT, a solution of 2,3-dichlorobenzene-sulphonylchloride (22.29 g, 90.8 mmol) in THF (80 mL) is added within 20 minutes. The reaction is stirred at reflux for 2 hours. After cooling, the solvent is removed under reduced pressure and the remaining solid suspended in 10% HCl (200 mL). The crude crystalline product is filtered-off, washed with H₂O and dried at 60° C. Final purification is done by suspending the crude compound in MeOH (250 mL), heating to reflux, filtration and drying. 2,3-Dichloro-N-(4-cyanomethyl-phenyl)-benzenesulfonamide (26.54 g, 86%) is obtained as orange crystals: m.p: 202-206° C.; (CH₂Cl₂/MeOH 98:2): 0.54; ES-MS [M−1]⁻=338.8; HPLC ^{B}t_{Ret}=5.85 minutes.

### Stage 10.3 4-(4-Dimethylamino-phenyl)-2H-pyrazol-3-ylamine

4-(4-Dimethylamino-phenyl)-2H-pyrazol-3-ylamine is prepared from 2-(4-dimethylamino-phenyl)-3-oxo-propionitrile (Stage 10.4) and hydrazine hydrate as described in U.S. Pat. No. 2,989,539 (20.6.61; Anderson and Reiff, Example 18). 4-(4-Dimethylamino-phenyl)-2H-pyrazol-3-ylamine: m.p. 173-176° C.; (CH₂Cl₂ MeOH/NH₃=90:10:1): 0.37; ES-MS [M+1]⁺=203; HPLC ^{B}t_{Ret}=1.40 minutes.

### Stage 10.4 2-(4-Dimethylamino-phenyl)-3-oxo-propionitrile

2-(4-Dimethylamino-phenyl)-3-oxo-propionitrile is prepared from (4-dimethylamino-phenyl)-acetonitrile, ethyl formate and sodium as described in U.S. Pat. No. 2,989,539 (Example 25).

2-(4-Dimethylamino-phenyl)-3-oxo-propionitrile: m.p. 175-178° C.; ES-MS [M+1]⁺=189; HPLC ^{B}t_{Ret}=2.00 minutes.

The compound of Example 18 is prepared analogously to the synthesis of the compound of Example 17 using 4-(4-dimethylamino-phenyl)-2H-pyrazol-3-ylamine (Stage 10.3) and 4-chloro-benzenesulfonic acid 4-(cyano-formyl-methyl)-phenyl ester (Stage 11.1).

### Stage 11.1 4-Chloro-benzenesulfonic acid 4-(cyano-formyl-methyl)-phenyl ester

4-Chloro-benzenesulfonic acid 4-(cyano-formyl-methyl)-phenyl ester is prepared as described in Example 17 (Stage 10.1), using commercially-available 4-(cyanomethyl)phenyl-4-chlorobenzene-1-sulfonate instead.

4-Chloro-benzenesulfonic acid 4-(cyano-formyl-methyl)-phenyl ester (162 mg); yellowish solid; (CH₂Cl₂/MeOH=95:2): 0.32; ES-MS [M+1]⁺=335; HPLC ^{B}t_{Ret}=6.23 minutes.

### Stage 12.1 2-(4-Methoxy-phenyl)-3-oxo-butyronitrile

2-(4-Methoxy-phenyl)-3-oxo-butyronitrile is prepared as described by Smith, Breen, Hajek and Awang, J. Org. Chem., Vol. 35, No. 7, pp. 2215-2221 (1970).

### Stage 14.1 2-(4-Bromo-phenyl)-3-oxo-butyronitrile

2-(4-Bromo-phenyl)-3-oxo-butyronitrile is synthesized according to the procedure of Rau, Ger. Offen., DE 3001266 (1980).

### Stage 16.1 1,2-(2,6-Dichloro-phenyl)-3-oxo-propionitrile

1,2-(2,6-Dichloro-phenyl)-3-oxo-propionitrile is prepared as described by Menzer, Lankau and Unverferth, Ger. Offen., DE 19521822 (1996).

### Stage 17.1 4-(3-Methoxy-phenyl)-2H-pyrazol-3-ylamine

4-(3-Methoxy-phenyl)-2H-pyrazol-3-ylamine and Stage 22.2 are prepared as described by Bruni et al., Heterocyclic. Chem., Vol. 32, No. 1, pp. 291-298 (1995).

### Stage 19.1 2-Benzo[b]thiophen-3-yl-3-oxo-propionitrile

2-Benzo[b]thiophen-3-yl-3-oxo-propionitrile is synthesized analogously to the preparation of compound of Stage 1.3: Yield: 56%; white powder; ES-MS: M−H=123.9; HPLC: ^{A}t_{Ret}=2.20 minutes.

¹H-NMR (300 MHz, DMSO-d₆): 12.0 (s/broad, 1H), 8.00-7.70 (m, 3H), 7.45-7.35 (m, 2H).

### Stage 21.1 3-Oxo-2-thiophen-3-yl-propionitrile

3-Oxo-2-thiophen-3-yl-propionitrile is synthesized analogously to the preparation of compound of Stage 1.3: Yield: 51%; white powder, ES-MS: M−H=112.9; HPLC: ^{A}t_{Ret}=2.03 minutes.

The compound forms a tautomeric equilibrium in solution: ¹H-NMR (300 MHz, DMSO-d₆): 7.95/7.55 (s/s, 1H, CH═/CH—OH), 7.55-7.50 (m, 2H), 7.30-7.20 (m, 1H).

### Stage 22.1 4-Benzo[b]thiophen-3-yl-1H-pyrazol-3-ylamine

4-Benzo[b]thiophen-3-yl-1H-pyrazol-3-ylamine is synthesized analogously to the preparation of compound of Stage 1.2: Yield: 80%; white powder; ES-MS: M+H=216.0.

¹H-NMR (300 MHz, DMSO-d₆): 12.0 (s/broad, 1H), 8.00-7.80 (m, 2H), 7.75 (s/broad, 1H), 7.60 (s/broad, 1H), 7.40-7.30 (m, 2H).

### Stage 22.2 (2-(3-Methoxy-phenyl)-3-oxo-propionitrile)

(2-(3-Methoxy-phenyl)-3-oxo-propionitrile) is prepared as described by Bruni et al., Heterocyclic. Chem., Vol. 32, No. 1, pp. 291-298 (1995).

### Stage 23.1 2-Formyl-3-phenyl-propionitrile

2-Formyl-3-phenyl-propionitrile is synthesized analogously to the preparation of compound of Stage 1.3: Yield: 77%; oil; ES-MS: M−H=158.0.

The compound forms a tautomeric equilibrium in solution: ¹H-NMR (300 MHz, DMSO-d₆): 7.0-7.15 (m, 5H), 2.85-2.75 (m, 2H).

### Stage 24.1 4[3-(4-Methyl-piperazin-1-yl)-phenyl]-acetonitrile

4[3-(4-Methyl-piperazin-1-yl)-phenyl]-acetonitrile is synthesized analogously to the preparation of compound of Stage 1.5: Yield 55%; brown solid; ES-MS: M+H=216.7; HPLC: ^{C}t_{Ret}=1.65 minutes.

¹H-NMR (300 MHz, CDCl₃): 7.30-7.25 (m, 1H), 6.90-6.82 (m, 2H), 6.80-6.75 (m, 1H), 3.70 (s, 2H), 3.25-3.15 (m, 4H), 2.60-2.50 (m, 4H), 2.35 (s, 3H).

### Stage 24.2 2-[3-(4-Methyl-piperazin-1-yl)-phenyl]-3-oxo-propionitrile

2-[3-(4-Methyl-piperazin-1-yl)-phenyl]-3-oxo-propionitrile is synthesized analogously to the preparation of compound of Stage 1.3: Yield: 100%; brown solid;

ES-MS: M+H=244.1; HPLC: ^{C}t_{Ret}=1.67 minutes.

### Stage 24.3 4-[3-(4-Methyl-piperazin-1-yl)-phenyl]-1H-pyrazol-3-ylamine

4-[3-(4-Methyl-piperazin-1-yl)-phenyl]-1H-pyrazol-3-ylamine is synthesized analogously to the preparation of compound of Stage 1.2: Yield: 36%; yellow foam; ES-MS: M+H=258.2; HPLC: ^{C}t_{Ret}=1.46 minutes.

¹H-NMR (300 MHz, CDCl₃): 7.45 (s, 1H), 7.30-7.25 (m, 1H), 7.05-7.00 (m, 1H), 6.95-6.90 (m, 1H), 6.85-6.80 (m, 1H), 4.00 (s/broad, 2H), 3.30-3.20 (m, 4H), 2.65-2.58 (m, 4H), 2.35 (s, 3H).

### Stage 25.1 2-(1-Methyl-1H-indol-3-yl)-3-oxo-propionitrile

2-(1-Methyl-1H-indol-3-yl)-3-oxo-propionitrile is synthesized analogously to the preparation of compound of Stage 1.3: Yield: 59%; oil; ES-MS: M+H=199.1.

The compound forms a tautomeric equilibrium in solution: ¹H-NMR (300 MHz, CDCl₃): 8.00/7.95 (s/s, 1H), 7.60-7.20 (m, 5H), 3.75 (s, 3H).

### Stage 26.1 2-(4-Methoxy-phenyl)-3-oxo-propionitrile

2-(4-Methoxy-phenyl)-3-oxo-propionitrile is synthesized analogously to the preparation of compound of Stage 1.3: Yield: 80%; white solid; ES-MS: M−H=174.3.

The compound forms a tautomeric equilibrium in solution: ¹H-NMR (300 MHz, DMSO-d₆): 7.80/7.58 (s/s, 1H), 7.55-7.50 (m, 1H), 7.30-7.20 (m, 1H), 6.90-6.80 (m, 2H), 3.73/3.70 (s/s, 3H).

### Stage 27.1 2-(2-Methoxy-phenyl)-3-oxo-propionitrile

2-(2-Methoxy-phenyl)-3-oxo-propionitrile is synthesized analogously to the preparation of compound of Stage 1.3: Yield: 40%; brown oil; ES-MS: M−H=174.3; HPLC ^{C}t_{Ret}=2.01 minutes.

### Stage 28.1 3-Oxo-2-pyridin-3-yl-propionitrile

3-Oxo-2-pyridin-3-yl-propionitrile is synthesized analogously to the preparation of compound of Stage 1.3: Yield: 71%%; brown solid; ES-MS: M+H=147.2; HPLC ^{C}t_{Ret}=1.31 minutes.

### Stage 28.2 4-Pyridin-3-yl-1H-pyrazol-3-ylamine

4-Pyridin-3-yl-1H-pyrazol-3-ylamine is synthesized analogously to the preparation of compound of Stage 1.2: Yield: 68%; brown solid; ES-MS: M+H=161.2; HPLC ^{C}t_{Ret}=0.50 minutes.

### Stage 30.1 [2-Methoxy-5-(4-methyl-piperazin-1-yl)-phenyl]-acetonitrile

[2-Methoxy-5-(4-methyl-piperazin-1-yl)-phenyl]-acetonitrile is synthesized analogously to the preparation of compound of Stage 1.5: Yield: 51%; brown solid; ES-MS: M+H=246.6; HPLC: ^{C}t_{Ret}=1.72 minutes.

¹H-NMR (300 MHz, CDCl₃): 7.00-6.95 (m, 1H), 6.85-6.75 (m, 2H), 3.80 (s, 3H), 3.65 (s, 2H), 3.15-3.05 (m, 4H), 2.60-2.55 (m, 4H), 2.35 (s, 3H).

### Stage 30.2 2-[2-Methoxy-5-(4-methyl-piperazin-1-yl)-phenyl]-3-oxo-propionitrile

2-[2-Methoxy-5-(4-methyl-piperazin-1-yl)-phenyl]-3-oxo-propionitrile is synthesized analogously to the preparation of compound of Stage 1.4: Yield: 100%; brown solid; ES-MS: M+H=274.1; HPLC: ^{C}t_{Ret}=1.62 minutes.

### Stage 30.3 4-[2-Methoxy-5-(4-methyl-piperazin-1-yl)-phenyl]-2H-pyrazol-3-ylamine

4-[2-Methoxy-5-(4-methyl-piperazin-1-yl)-phenyl]-2H-pyrazol-3-ylamine is synthesized analogously to the preparation of compound of Stage 1.2: Yield: 32%; brown solid; ES-MS: M+H=288.2; HPLC: ^{C}t_{Ret}=1.46 minutes.

¹H-NMR (300 MHz, CDCl₃): 7.50 (s, 1H), 7.00-6.95 (m, 1H), 6.90-6.80 (m, 2H), 3.80 (s, 3H), 3.20-3.10 (m, 4H), 2.65-2.55 (m, 4H), 2.35 (s, 3H).

### Stage 32.1 2-(2-Benzyloxy-phenyl)-3-oxo-propionitrile

2-(2-Benzyloxy-phenyl)-3-oxo-propionitrile is synthesized analogously to the preparation of compound of Stage 1.3: Yield: 85%; white solid; ES-MS: M+H=252.6; HPLC: ^{C}t_{Ret}=2.35 minutes.

The compound forms a tautomeric equilibrium in solution: ¹H-NMR (300 MHz, DMSO-d₆): 11.6/7.78 (s, 1H), 7.55-7.45 (m, 2H), 7.40-7.20 (m, 5H), 7.15-7.05 (m, 1H), 7.00-6.90 (m, 1H), 5.15 (s, 2H).

### Stage 34.1 2-(4-Benzyloxy-phenyl)-3-oxo-propionitrile

2-(4-Benzyloxy-phenyl)-3-oxo-propionitrile is synthesized analogously to the preparation of compound of Stage 1.3: Yield: 95%; white solid; ES-MS: M−H=250.3; HPLC: ^{C}t_{Ret}=2.41 minutes.

The compound forms a tautomeric equilibrium in solution: ¹H-NMR (300 MHz, DMSO-d₆): 12.0/11.7 (s, 1H), 7.90-7.80 and 7.60-7.50 (m, 1H), 7.40-7.25 (m, 6H), 7.05-6.95 (m, 2H), 5.10 (s, 2H).

### Stage 44.1 4-(1-Methyl-1H-indol-3-yl)-2H-pyrazol-3-ylamine

4-(1-Methyl-1H-indol-3-yl)-2H-pyrazol-3-ylamine is synthesized analogously to the preparation of compound of Stage 1.2: Yield: 10%; brown foam; ES-MS: M+H=213.2; HPLC: ^{C}t_{Ret}=1.66 minutes.

¹H-NMR (300 MHz, DMSO-d₆): 7.70 (d, 1H), 7.60 (s, 1H), 7.35 (d, 1H), 7.30-7.25 (m, 1H), 7.20-7.10 (m, 2H), 3.80 (s, 3H).

### Stage 47.1 2-(2-Methoxy-phenyl)-3-oxo-propionitrile

2-(2-Methoxy-phenyl)-3-oxo-propionitrile is synthesized analogously to the preparation of compound of Stage 1.3: Yield: 59%; white solid; ES-MS: M+H=175.3; HPLC: ^{C}t_{Ret}=2.01 minutes.

### Stage 47.2 4-(2-Methoxy-phenyl)-2H-pyrazol-3-ylamine

4-(2-Methoxy-phenyl)-2H-pyrazol-3-ylamine is synthesized analogously to the preparation of compound of Stage 1.2: Yield: 35%; white solid; ES-MS: M+H=190.1; HPLC: ^{C}t_{Ret}=1.40 minutes.

¹H-NMR (300 MHz, DMSO-d₆): 11.5 (bs, 1H), 7.50 (bs, 1H), 7.30 (bs, 1H), 7.20-7.05 (m, 1H), 7.00-6.85 (m, 2H), 4.30 (bs, 2H), 3.75 (s, 3H).

### Stage 51.1 3-Oxo-2-pyridin-4-yl-propionitrile

3-Oxo-2-pyridin-4-yl-propionitrile is synthesized analogously to the preparation of compound of Stage 1.3: Yield: 59%; orange solid; ES-MS: M+H=147.2; HPLC: ^{C}t_{Ret}=1.00 minute.

The compound forms a tautomeric equilibrium in solution: ¹H-NMR (300 MHz, DMSO-d₆): 13.1/9.60 (bs, 1H), 9.10 (bs, 1H), 8.20-8.00 (m, 2H), 7.95-7.80 (m, 1H).

### Stage 52.1 (Z)-3-Dimethylamino-2-(3-nitro-phenyl)-acrylonitrile

(3-Nitro-phenyl)-acetonitrile (1.51 g, 9.31 mmol), dimethoxymethyl-dimethyl-amine (6.2 mL, 46.5 mmol) in xylene (30 mL) are stirred at reflux for 1 hour. After adding hexane (20 mL), the reaction mixture is cooled at 0° C. Precipitating material is filtered-off to give compound of Stage 52.1 as brown solid (1.76 g, 8.19 mmol; 88%); ES-MS: M+H=218.1; HPLC: ^{C}t_{Ret}=2.24 minutes.

¹H-NMR (300 MHz, DMSO-d₆): 8.10-8.05 (m, 1H), 7.90-7.85 (m, 1H), 7.75-7.72 (r, 1H), 7.70 (s, 1H), 7.65-7.60 (m, 1H), 3.30 (s, 6H).

### Stage 52.2 3-[3-(4-Methyl-piperazin-1-yl)-phenyl]-6-(3-nitro-phenyl)-pyrazolo[1,5-a]pyrimidin-7-ylamine

4-[3-(4-Methyl-piperazin-1-yl)-phenyl]-1H-pyrazol-3-ylamine (Stage 24.3) (305 mg, 1.18 mmol), (Z)-3-dimethylamino-2-(3-nitro-phenyl)-acrylonitrile (Stage 52.1) (335 mg, 1.54 mmol) dissolved in AcOH (10 mL) and BuOH (10 mL) are stirred at reflux for 16 hours. After adding saturated NaHCO₃ aqueous solution, the reaction mixture is extracted with EtOAc (50 mL, 2×). The combined organic phases are washed with H₂O (10 mL), dried (Na₂SO₄), concentrated under reduced pressure and flash chromatographed (silica gel, 2.5×15 cm, CH₂Cl₂/MeOH=9:1) to give compound of Stage 52.2 as orange solid (224 mg, 0.52 mmol; 44%); ES-MS: M+H=430.1; HPLC: ^{C}t_{Ret}=1.91 minutes.

¹H-NMR (300 MHz, DMSO-d₆): 8.70 (s, 1H), 8.35-8.30 (m, 1H), 8.25 (s, 1H), 8.22-8.18 (m, 1H), 7.98-7.95 (m, 1H), 7.90 (bs, 2H), 7.80-7.70 (m, 2H), 7.65-7.60 (m, 1H), 7.25-7.18 (m, 1H), 6.80-6.75 (m, 1H), 3.20-3.10 (m, 4H), 2.50-2.40 (m, 4H), 2.20 (s, 3H).

### Stage 53.1 3-[4-(4-Methyl-piperazin-1-yl)-phenyl]-6-(3-nitro-phenyl)-pyrazolo[1,5-a]pyrimidin-7-ylamine

3-[4-(4-Methyl-piperazin-1-yl)-phenyl]-6-(3-nitro-phenyl)-pyrazolo[1,5-a]pyrimidin-7-ylamine is synthesized analogously to the preparation of compound of Stage 52.2: Yield: 30%; red solid; ES-MS: M+H=430.0.

¹H-NMR (300 MHz, DMSO-d₆): 8.60 (s, 1H), 8.35-8.30 (m, 1H), 8.22 (s, 1H), 8.20-8.10 (m, 1H), 8.00 (d, 2H, J=7.9 Hz), 7.95-7.90 (m, 1H), 7.85 (bs, 2H), 7.80-7.75 (m, 1H), 6.95 (d, 1H, J=7.9 Hz), 3.20-3.10 (m, 4H), 2.50-2.40 (m, 4H), 2.20 (s, 3H).

### Stage 54.1 (Z)-3-Dimethylamino-2-(2-nitro-phenyl)-acrylonitrile

(Z)-3-Dimethylamino-2-(2-nitro-phenyl)-acrylonitrile is synthesized analogously to the preparation of compound of Stage 52.1: Yield: 97%; brown solid.

¹H-NMR (300 MHz, DMSO-d₆): 7.82-7.78 (m, 1H), 7.62-7.55 (m, 1H), 7.45-7.35 (m, 2H), 7.20 (s, 1H), 3.15 (s, 6H).

### Stage 54.2 3-[4-(4-Methyl-piperazin-1-yl)-phenyl]-6-(2-nitro-phenyl)-pyrazolo[1,5-a]pyrimidin-7-ylamine

3-[4-(4-Methyl-piperazin-1-yl)-phenyl]-6-(2-nitro-phenyl)-pyrazolo[1,5-a]pyrimidin-7-ylamine is synthesized analogously to the preparation of compound of Stage 55.2: brown solidl; ES-MS: M+H=430.0; HPLC: ^{D}t_{Ret}=1.61 minutes.

¹H-NMR (300 MHz, DMSO-d₆): 8.55 (s, 1H), 8.20-8.15 (m, 1H), 8.05-7.95 (m, 3H), 7.82-7.60 (m, 5H), 7.00-6.95 (m, 2H), 3.15-3.05 (m, 4H), 2.45-2.40 (m, 4H), 2.20 (s, 3H).

### Stage 55.1 (E)-3-Dimethylamino-2-(4-methyl-thiazol-2-yl)-acrylonitrile

(E)-3-Dimethylamino-2-(4-methyl-thiazol-2-yl)-acrylonitrile is synthesized analogously to the preparation of compound of Stage 52.1: Yield: 74%; black solid; ES-MS: M+H=194.2; HPLC: ^{C}t_{Ret}=1.57 minutes.

¹H-NMR (300 MHz, DMSO-d₆): 7.76 (s, 1H), 6.60 (s, 1H), 3.25 (bs, 6H), 2.35 (s, 3H).

### Example 68

### 3-{7-Amino-2-methyl-3-[4-(4-methyl-piperazin-1-yl)phenyl]-pyrazolo[1,5-a]pyrimidin-6-yl}-phenol

3-{7-Amino-2-methyl-3-[4-(4-methyl-piperazin-1-yl)phenyl]-pyrazolo[1,5-a]pyrimidin-6-yl}-phenol is synthesized analogously to the preparation of Example 1 by using methyl hydrazine instead of hydrazine when the pyrazole ring is formed: ES-MS: M+H=415.2; HPLC: ^{D}t_{Ret}=1.45 minutes.

¹H-NMR (300 MHz, DMSO-d₆): 9.53 (s, 1H, OH), 2.56 (s, 3H CH₃), 2.24 (s, 3H CH₃).

### Example 69

### (4-{7-Amino-3-[4-(4-methyl-piperazin-1-yl)-phenyl]-pyrazolo[1,5-a]pyrimidin-6-yl}-phenyl)-carbamic acid ethyl ester

4-(4-(4-Methyl-piperazin-1-yl)-phenyl)-2H-pyrazol-3-ylamine (Stage 1.2) (200 mg, 0.81 mmol) and [4-(2-cyano-1-formyl-ethyl)-phenyl]-carbamic acid ethyl ester (Stage 62.1) (275 mg, 0.04 mmol) dissolved in EtOH (4 mL) and ethanolic HCl (1.6 mL, 2.5 N) are stirred under reflux for 17 hours under Ar. After adding H₂O (4 mL) and K₂CO₃ (250 mg), the reaction mixture is extracted with CH₂Cl₂ (20 mL, 2×). The combined organic phases are washed with H₂O (10 mL), dried (Na₂SO₄), concentrated under reduced pressure and flash chromatographed (silica gel, 2.5×15 cm, CH₂Cl₂/MeOH/NH₃=95:5:0.5) to give compound of Example 62 as white solid (58 mg, 0.123 mmol; 15%); ES-MS: M+H=472.0; R_{f} (CH₂Cl₂/MeOH/NH₃=90:10:0.1) 0.42; HPLC: ^{A}t_{Ret}=4.26 minutes.

¹H-NMR (400 MHz, DMSO-d₆): 8.75/8.58 (s/s, 1H/1H, pyrazolopyrimidinyl), 8.03 (d, 9.0 Hz, 2H, phenyl), 7.61 (d, 9 Hz, 2H, phenyl), 7.53 (s, 2H, NH₂), 7.46 (d, 9 Hz, 2H, phenyl), 7.00 (d, 9 Hz, 2H, phenyl), 4.17 (q, 7.5 Hz, 2H, CH₂-Ethyl), 3.17/2.48 (m/m, 4H/4H, piperazinyl), 2.24 (t, 7.5 Hz, 3H, CH₃).

### Stage 62a. 1[4-(Cyano-1-formyl-methyl)-phenyl]-carbamic acid ethyl ester

[4-(Cyano-methyl)-phenyl]-carbamic acid benzyl ester (Stage 62a.2) (1 g, 3.76 mmol) is formylated in analogy to the preparation of Stage 1.3 giving the corresponding carbamic acid ethyl ester (thereby also transforming the benzyl ester function into the ethyl ester function): colorless crystals (654 mg, 2.66 mmol, 701%). ES-MS: M+H=233.0.

¹H-NMR (400 MHz, DMSO-d₆): 4.12 (q/broad, 7.5 Hz, 2H, CH₂-Ethyl), 1.23 (t/broad, 7.5 Hz, 3H, CH₃-Ethyl).

### Stage 62.2 [4-(Cyano-methyl)-phenyl]-carbamic acid benzyl ester

(4-Amino-phenyl)-acetonitrile (2 g, 15.1 mmol) and dibenzyl dicarbonate (4.33 g, 15.1 mmol) dissolved in dioxane (16 mL) are stirred for 1 hour at RT. After evaporating the solvent, the product is isolated by flash chromatography (silica gel, 4.5×25 cm, CH₂Cl₂/MeOH=99:1): white solid (3.82 g, 14.4 mmol; 95%); ES-MS: M−H=265.0; R_{f} (CH₂Cl₂/MeOH=95:5)=0.49; HPLC: ^{A}t_{Ret}=6.32 minutes.

¹H-NMR (400 MHz, DMSO-d₆): 9.82 (s, 1H, NH), 7.51-7.35 (m, 7H, aryl), 7.26 (d, 8.5 Hz, 2H, aryl), 5.15 (s, 2H, CH₂), 3.95 (s, 2H, CH₂).

### (Z-)-3-Dimethylamino-2-thiazol-4-yl-acrylonitrile

(Z-)-3-Dimethylamino-2-thiazol-4-yl-acrylonitrile is synthesized analogously to the preparation of compound of Stage 52.1: ES-MS [M+1]⁺=180.1; HPLC: ^{C}t_{Ret}=1.91 minutes

Compounds 68, 69, 71, 74, and 75 carrying sulfonamide and acetylamide functions (compounds 50, 72 and 76) are prepared by reacting the amino precursor with the corresponding sulfonic acid chloride or acetic acid anhydride in the presence of pyridine.

### Examples 77 and 78

The compounds in Table 2 and Table 3 are prepared according to Example 8.

TABLE 2

| | |
|---|---|
| | Example 77 |
| 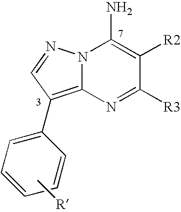 | |

| Nb. | R′ | R2 | R3 |
|---|---|---|---|
| A | 4-(4-Methyl-piperazin-1-yl) | 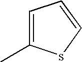 | H |
| B | 4-(—O—(CH₂)₂—NH₂) | 3-Hydroxyphenyl | H |
| C | 4-(—O—(CH₂)₂—NH₂) | H | 4-pyridinyl |
| D | 4-(4-Methyl-piperazin-1-yl) | 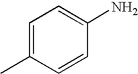 | H |
| E | 4-(4-Methyl-piperazin-1-yl) | 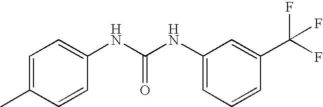 | H |
| F | 4-(4-Methyl-piperazin-1-yl)/2-Cl | 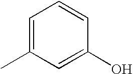 | H |
| G | 4-Dimethylaminyl | 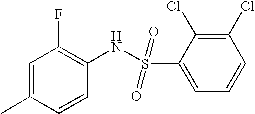 | H |
| H | 4-(4-Methyl-piperazin-1-yl) | 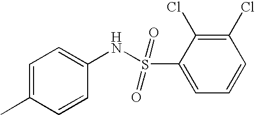 | H |
| I | 4-Dimethylaminyl | 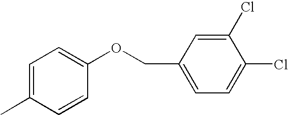 | H |
| K | 4-(4-Methyl-piperazin-1-yl) | 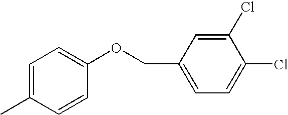 | H |
| L | 4-(4-Methyl-piperaizn-1-yl) | 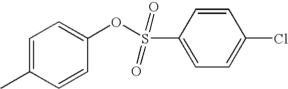 | H |
| M | 4-(4-Methyl-piperazin-1-yl) | H | 4-Pyridinyl |
| N | 4-(4-Methyl-piperazin-1-yl) | 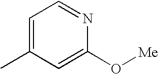 | H |
| O | 4-(4-Methyl-piperazin-1-yl) | 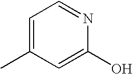 | H |
| R | 4-(4-Methyl-piperazin-1-yl) | 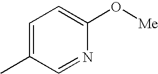 | H |
| S | 4-(4-Methyl-piperazin-1-yl) | 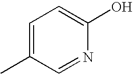 | H |
| V | 4-(4-Methyl-piperazin-1-yl) | H | 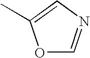 |
| W | 4-(4-Methyl-piperazin-1-yl)/2-Methoxy | 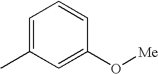 | H |
| X | 4-(4-Methyl-piperazin-1-yl)/2-Methoxy | 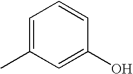 | H |
| Y | 4-(4-Methyl-piperazin-1-yl) | 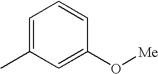 | H |
| Z | 4-(4-Methyl-piperazin-1-yl) | 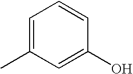 | H |
| z1 | 3-(4-Methyl-piperazin-1-yl) | 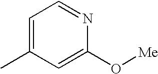 | H |
| z2 | 3-(4-Methyl-piperazin-1-yl) | 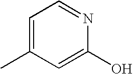 | H |
| z3 | 3-(4-Methyl-piperazin-1-yl) | 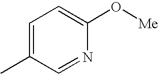 | H |
| z4 | 3-(4-Methyl-piperazin-1-yl) | 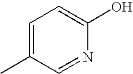 | H |
| z5 | 3-(4-Methyl-piperazin-1-yl) | H | 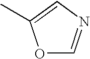 |
| Z6 | 4-(4-Methyl-piperazin-1-yl) | 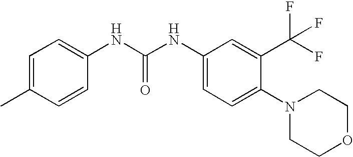 | H |
| Z7 | 3-(4-Methyl-piperazin-1-yl) | 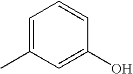 | CH₃ |
| Z8 | 3-(4-Methyl-piperazin-1-yl) | 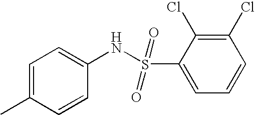 | H |
| Z9 | 4-(4-Methyl-piperazin-1-yl) | 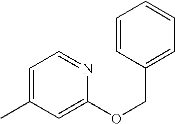 | H |
| z10 | 4-(4-Methyl-piperazin-1-yl) | 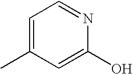 | H |
| z11 | 3-(4-Methyl-piperazin-1-yl) | 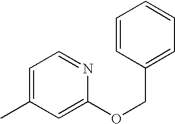 | H |
| z12 | 3-(4-Methyl-piperazin-1-yl) | 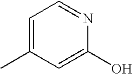 | H |
| z13 | 5-(4-Methyl-piperazin-1-yl)/2-methoxy | 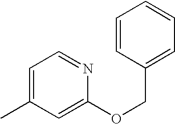 | H |
| z14 | 5-(4-Methyl-piperazin-1-yl)/2-methoxy | 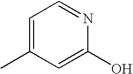 | H |
| z15 | 4-(4-Methyl-piperazin-1-yl)/2-methoxy | 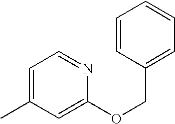 | H |
| z16 | 4-(4-Methyl-piperazin-1-yl)/2-methoxy | 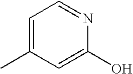 | H |
| z17 | 4-(4-Methyl-piperazin-1-yl) | Br | H |
| z18 | 3-(4-Methyl-piperazin-1-yl) | 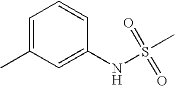 | H |
| Z19 | 4-(4-Methyl-piperazin-1-yl)/2- | 3-Benzoyloxyphenyl | H |
| | Methoxy | | |
| z20 | 4-(4-Methyl-piperazin-1-yl)/2- | 3-Hydroxyphenyl | H |
| | Methoxy | | |
| z21 | 3-(4-Methyl-piperazin-1-yl) | 3-Chlorophenyl | Me |
| z22 | 4-(4-Methyl-piperazin-1-yl)/2- | 3-Chlorophenyl | Me |
| | methoxy | | |
| Z23 | 5-(4-Methyl-piperazin-1-yl)/2- | 3-Chlorophenyl | Me |
| | methoxy | | |

TABLE 3

| | | | Example 78 |
|---|---|---|---|
| 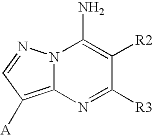 | | | |
| Nb. | A | R2 | R3 |
| A | Br | H | 4-Pyridinyl |
| B | H | H | 4-Pyridinyl |
| C | Br | 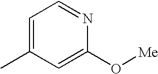 | H |
| D | Br | 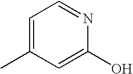 | H |
| E | Br | 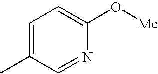 | H |
| F | Br | 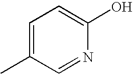 | H |
| G | H | 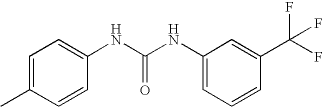 | H |
| H | Pyridin-4-yl | 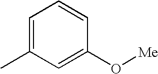 | H |

### Example 79

### 6-(3-Chloro-phenyl)-5-methyl-3-[3-(4-methyl-piperazin-1-yl)-phenyl]-pyrazo-1o[1,5-a]pyrimidin-7-ylamine

4-[3-(4-Methyl-piperazin-1-yl)-phenyl]-1H-pyrazol-3-ylamine (Stage 72.2) (1.29 g, 5 mmol), is dissolved in EtOH (25 mL), followed by the addition of 2-(3-Chloro-phenyl)-3-oxo-butyronitrile (Stage 72.3) (0.97 g, 5 mmol) and HCl (1.25 M in EtOH; 20 mmol, 16 mL) at RT. The yellowish solution is refluxed under stirring for 20 h. After cooling to RT, H₂O (80 mL) is added as well as K₂CO₃ (2.5 g) to render the mixture basic. The aqueous layer is extracted with CH₂Cl₂ (200 mL, 2.times.). The combined organic phases are washed with H₂O (50 mL, 2.times.), dried (Na₂SO₄), concentrated under reduced pressure and chromatographed (silica gel, 120 g RediSep, ISCO Sg-100 CH₂Cl₂/MeOH/NH₃=95:5:0.1) to obtain the title compound 72 as white crystals (1.03 g, 2.38 mmol; 48%); mp. 10-115° C.; MS(ESI+):m/z=433 (M+H)⁺; HPLC: ^{A}t_{RET}=3.72 minutes (System1).

### Stage 72.1: 2-(3-Chloro-phenyl)-3-oxo-butyronitrile

355 ml of ethanol is heated to 55° C. under N2. To this solution is added sodium (3.91 g; 0.17 mol) within 30 min. and stirred for 1.5 h until all metal is dissolved. 3-Chlorobenzyl cyanide (15.31 g; 0.1 mol) and ethyl acetate (28.53 mL; 0.29 mol) are added to the colorless solution, followed by stirring under reflux for 5 h. After completion of the reaction, the yellow mixture is cooled to rt. and evaporated under reduced pressure. The crude material is taken up into water (200 mL) and neutralized by addition of 25 g of citric acid. The aqueous layer is extracted with CH₂Cl₂ (2.times.250 mL). The combined organic phases are washed with H₂O (2.times.150 mL,), dried (Na₂SO₄), concentrated under reduced pressure and chromatographed (silica gel, 1 kg, Merck 60 (0.040-0.063), eluting with EtOAc/Hexanes 1:1) to obtain the title compound 72.1 as yellowish crystals (9.7 g, 0.05 mol; 50%); mp. 92-97° C.; MS(ESI+):m/z=302.9 (M+H)⁺; HPLC: ^{A}t_{RET}=5.67 minutes (System1).

### Stage-72.2: 4-[3-(4-Methyl-piperazin-1-yl)-phenyl]-1H-pyrazol-3-ylamine

The title compound is prepared as described in example 31; Stage 24.1-24.3

### Example 80

### 6-(3-Chloro-phenyl)-5-methyl-3-[4-(4-methyl-piperazin-1-yl)-phenyl]-pyrazo-1o[1,5-a]pyrimidin-7-ylamine

The title compound is prepared as described in example 86; using 4-[4-(4-Methyl-piperazin-1-yl)-phenyl]-2H-pyrazol-3-ylamine (Example 8; Stage 1.2) and 2-(3-Chloro-phenyl)-3-oxo-butyronitrile (Example 80, Stage 73.1) instead. Beige crystals; mp. 113-115° C.; MS(ESI+):m/z=433 (M+H)⁺; HPLC: ^{A}t_{RET}=3.56 minutes (System1);

### Example 81

### 6-(3-Chloro-phenyl)-3-[2-methoxy-5-(4-methyl-piperazin-1-yl)-phenyl]-5-met-hyl-pyrazolo[1,5-a]pyrimidin-7-ylamine

The title compound is prepared as described in example 86; using 4-[2-Methoxy-5-(4-methyl-piperazin-1-yl)-phenyl]-2H-pyrazol-3-ylamine and 2-(3-Chloro-phenyl)-3-oxo-butyronitrile (Example 79, Stage 72.1) instead. Beige crystals; mp. 116-121° C.; MS(ESI+):m/z=463 (M+H)⁺; HPLC: ^{A}t_{RET}=3.68 minutes (System1).

### Stage 74.1: 4-[2-Methoxy-5-(4-methyl-piperazin-1-yl)-phenyl]-2H-pyrazol-3-ylamine

The title compound is prepared as described in example 8, (Stage 1.2; Stage 1.4 and 1.5); using 5-Bromo-2-methoxy-phenylacetonitrile and N-methylpiperazine instead. Yellowish foam; MS(ESI+):m/z=288.2 (M+H)⁺; HPLC: ^{A}t_{RET}=3.53 minutes (System2).

### Example 82

### 6-(3-Chloro-phenyl)-3-[2-methoxy-4-(4-methyl-piperazin-1-yl)-phenyl]-5-met-hyl-pyrazolo[1,5-a]pyrimidin-7-ylamine

The title compound is prepared as described in example 86; using 4-[2-Methoxy-4-(4-methyl-piperazin-1-yl)-phenyl]-2H-pyrazol-3-ylamine and 2-(3-Chloro-phenyl)-3-oxo-butyronitrile (Example 79, Stage 72.1) instead. Beige crystals; mp. 215-217° C.; MS(ESI+):m/z=463 (M+H)⁺; HPLC: ^{A}t_{RET}=3.63 minutes (System1).

Stage 75.1: 4-[2-Methoxy-5-(4-methyl-piperazin-1-yl)-phenyl]-2H-pyrazol-3-ylamine. The title compound is prepared as described in example 8, (Stage. 1.2; Stage 1.4 and 1.5); using 4-Bromo-2-methoxy-phenylacetonitril-e and N-methylpiperazine instead. Green-brown crystals; mp. 173.7-178.1° C.; MS(ESI+):m/z=288.1 (M+H)⁺; HPLC: ^{A}t_{RET}=3.40, minutes (System 2).

### Example 83

### 3-{7-Amino-3-[2-methoxy-4-(4-methyl-piperazin-1-yl)-phenyl]-pyrazolo[1,5-a-]pyrimidin-6-yl}-phenol

The title compound is prepared by dissolving 6-(3-Benzyloxy-phenyl)-3-[2-methoxy-4-(4-methyl-piperazin-1-yl)-phenyl]-p-yrazolo[1,5-a]pyrimidin-7-ylamine in methanol and subjecting it to catalytic hydrogenation in the presence of Pd/C as described in example 1.: Beige crystals; mp. 217-220° C.;

MS(ESI+):m/z=431.0 (M+H)⁺; HPLC: ^{A}t_{RET}=2.65 minutes (System1).

### Stage 76.1: 6-(3-Benzyloxy-phenyl)-3-[2-methoxy-4-(4-methyl-piperaz-in-1-yl)-phenyl]-pyrazolo[1,5-a]pyrimidin-7-ylamine

The title compound is prepared as described in example 8, (Stage 1.2; Stage 1.4 and 1.5); using 4-Bromo-2-methoxy-phenylacetonitrile and N-methylpiperazine instead. Yellowish solid; MS(ESI+):m/z=521 (M+H)⁺; HPLC: ^{A}t_{RET}=4.38 minutes (System 1).

### Example 84

### 6-(2-Chloro-phenyl)-3-[4-(4-methyl-piperazin-1-yl)-phenyl]-pyrazolo[1,5-a]-pyrimidin-7-ylamine

The title compound is prepared as described in example 86; using 4-[4-(4-Methyl-piperazin-1-yl)-phenyl]-2H-pyrazol-3-ylamine and (Z)-2-(2-Chloro-phenyl)-3-dimethylamino-acrylonitrile instead. Yellow solid; mp. 197-200° C.; MS(ESI+):m/z=419 (M+H)⁺; HPLC: ^{A}t_{RET}=3.33 minutes (System1).

### Stage 77.1: (Z)-2-(2-Chloro-phenyl)-3-dimethylamino-acrylonitrile

N,N-Dimethylformamide-dimethylacetal (9.06 mL; 64.3 mMol) and 2-chlorobenzylcyanide (1.95 g; 12.86 mMol) is heated under stirring to 100° C. under an atmosphere of Argon. After cooling to rt, the mixture is concentrated under reduced pressure and purified by and chromatography (silica gel, 120 g RediSep, ISCO Sg-100, eluting with EtOAc/hexanes 1:1) to obtain the title compound as yellow thick oil (2.44 g, 11.8 mmol; 92%); MS(ESI+):m/z=207 (M+H)⁺; TLC (EtOAc/hexanes 1:1) R_{f}=0.38.

### Example 85

### 6-(2-Chloro-phenyl)-3-[3-(4-methyl-piperazin-1-yl)-phenyl]-pyrazolo[1,5-a]-pyrimidin-7-ylamine

The title compound is prepared as described in example 86; using (Z)-2-(2-Chloro-phenyl)-3-dimethylamino-acrylonitrile (Example 84, Stage 77.1) instead. Yellowish crystals; mp. 200-203° C.; MS(ESI+):m/z=419.0 (M+H)⁺; HPLC: ^{A}t_{RET}=3.65 minutes (System1).

### Example 86

### 6-(4-Fluoro-phenyl)-5-methyl-3-[4-(4-methyl-piperazin-1-yl)-phenyl]-pyrazo-1o[1,5-a]pyrimidin-7-ylamine

The title compound is prepared as described in example 86; using 4-[4-(4-Methyl-piperazin-1-yl)-phenyl]-2H-pyrazol-3-ylamine and 2-(4-Fluoro-phenyl)-3-oxo-butyronitrile instead. White crystals; mp. 289-291° C.; MS(ESI⁺):m/z=417.1 (M+H)⁺; HPLC: ^{A}t_{RET}=3.21 minutes (System1).

### Stage 79.1: 2-(4-Fluoro-phenyl)-3-oxo-butyronitrile

The title compound is prepared as described for example 79, Stage 72.1 using (4-Fluoro-phenyl)-acetonitrile instead. Beige crystals; mp. 77-83° C.; MS(ESI⁺):m/z=176.9 (M+H)⁺; HPLC: ^{A}t_{RET}=5.15 minutes (System1).

### Example 87

### 6-(4-Fluoro-phenyl)-5-methyl-3-[3-(4-methyl-piperazin-1-yl)-phenyl]-pyrazo-1o[1,5-a]pyrimidin-7-ylamine

The title compound is prepared as described in example 86; using 2-(4-T Fluoro-phenyl)-3-oxo-butyronitrile (Example 86, Stage 79.1) instead. White crystals; mp. 204-206° C.; MS(ESI+):m/z=417.1 (M+H)⁺; HPLC: ^{A}t_{RET}=3.34 minutes (System1).

### Example 88

### 6-(3-Chloro-phenyl)-5-methyl-3-{3-[4-(1-methyl-piperidin-4-yl)-piperazin-1-yl]-phenyl}-pyrazolo[1,5-a]pyrimidin-7-ylamine

The title compound is prepared as described in example 86; using 4-{3-[4-(1-Methyl-piperidin-4-yl)-piperazin-1-yl]-phenyl)-2H-pyrazol-3-yl-amine instead. Beige crystals; mp. 180-185° C.; MS(ESI+):m/z=516.0 (M+H)⁺; HPLC: ^{A}t_{RET}=4.96 minutes (System1).

### Stage 81.1: 4-{3-[4-(1-Methyl-piperidin-4-yl)-piperazin-1-yl]-phenyl}-2H-p-yrazol-3-ylamine

The title compound is prepared as described in example 8, (Stage 1.2 and 1.4 and 1.5); using (3-Bromo-phenyl)-acetonitrile and 1-(1-Methyl-piperidin-4-yl)-piperazine instead. Yellowish crystals; mp. 213-220° C.; MS(ESI+): m/z=341.18 (M+H)⁺; HPLC: ^{A}t_{RET}=3.57 minutes (System1).

### Example 89

### 6-(3-Chloro-4-fluoro-phenyl)-5-methyl-3-[3-(4-methyl-piperazin-1-yl)-pheny-1]pyrazolo[1,5-a]pyrimidin-7-ylamine

The title compound is prepared as described in example 86; using 2-(3-Chloro-4-fluoro-phenyl)-3-oxo-butyronitrile instead. White crystals; mp. 224-226° C.; MS(ESI+):m/z=451 (M+H)⁺; HPLC: ^{A}t_{RET}=3.86 minutes (System1).

### Stage 82.1: 2-(3-Chloro-4-fluoro-phenyl)-3-oxo-butyronitrile

The title compound is prepared as described for example 79, Stage 72.1 using (3-Chloro-4-fluoro-phenyl)-acetonitrile instead. White crystals; mp. 133-134° C.; MS(ESI):m/z=209.9 (M−H)⁻; HPLC: ^{A}t_{RET}=5.79 minutes (System1).

### Example 90

### 6-(3-Chloro-4-fluoro-phenyl)-5-methyl-3-[4-(4-methyl-piperazin-1-yl)-pheny-1]-pyrazolo[1,5-a]pyrimidin-7-ylamine

The title compound is prepared as described for example 79, using 4-[4-(4-Methyl-piperazin-1-yl)-phenyl]-2H-pyrazol-3-ylamine and 2-(3-Chloro-4-fluoro-phenyl)-3-oxo-butyronitrile (Example 89; stage 82.1) instead. White crystals; mp. 264-265° C.; MS(ESI+):m/z=451 (M+H)⁺; HPLC: ^{A}t_{Ret}=3.72 minutes (System1).

### Example 91

### 6-(3-Bromo-phenyl)-5-methyl-3-[3-(4-methyl-piperazin-1-yl)-phenyl]-pyrazol-o[1,5-a]pyrimidin-7-ylamine

The title compound is prepared as described for example 79, Stage 72.1 using 2-(3-Bromo-phenyl)-3-oxo-butyronitrile instead. White crystals; mp. 107-113° C.; MS(ESI+):m/z=477 (M+H)⁺; HPLC: ^{A}t_{RET}=4.90 minutes (System1);

### Stage 84.1: 2-(3-Bromo-phenyl)-3-oxo-butyronitrile

The title compound is prepared as described for example 79, Stage 72.1 using (3-Bromo-phenyl)-acetonitrile instead. White crystals; mp. 96-100° C.; MS(ESI⁻):m/z=235.9 (M−H)⁻; HPLC: ^{A}t_{RET}=5.76 minutes (System1).

### Example 92

### 6-(3-Bromo-benzyl)-3-[3-(4-methyl-piperazin-1-yl)-phenyl]pyrazolo[1,5-a]py-rimidin-7-ylamine

The title compound is prepared as described in example 86; using 3-(3-Bromo-phenyl)-2-formyl-propionitrile instead. White crystals; mp. 170-171° C.; MS(ESI+):m/z=477.0 (M+H)⁺; HPLC: ^{A}t_{RET}=3.84 minutes (System1).

### Stage 85.1: 3-(3-Bromo-phenyl)-2-formyl-propionitrile

3-(3-Bromophenyl)propionitrile (0.703 mL, 4.66 mMol) and ethyl formate (1.499 mL; 18.64 mMol) are dissolved in THF anhydrous (12.5 mL) followed by the addition of NaH (60% in mineral oil, 670 mg) at rt. After 17 h at rt, additional NaH (448 mg) and ethyl formate (0.765 mL) is added. Since this results in a strong exothermic reaction, additional solvent is added (15 mL of THF). After completion (3 days), the reaction mixture is cooled to 0° C., treated with a few little ice cubes, followed by addition of 6N HCl (3 mL) to acidify the mixture. After addition of water (50 mL), the mixture is extracted with EtOAc (3.times.100 mL). The combined organic phases are washed with H₂O (50 mL, 2.times.), brine, dried (Na₂SO₄), concentrated under reduced pressure and chromatographed (silica gel, 40 g RediSep, ISCO Sg-100, eluting with EtOAc/hexanes 1:1) to obtain the title compound as a brownish oil (220 mg; 20%); MS(ESI−):m/z=235.9 (M−H)⁻; TLC EtOAc/hexanes 1:1) Rf=0.28.

### Example 93: 6-(3-Bromo-phenyl)-3-[3-(4-methyl-piperazin-1-yl)-phenyl]-pyrazolo[1,5-a]p-yrimidin-7-ylamine

The title compound is prepared as described in example 86; using (Z)-2-(3-Bromo-phenyl)-3-dimethylamino-acrylonitrile instead. White crystals; mp. 195.3-197.2° C.; MS(ESI⁺):m/z=463.0 (M+H)⁺; HPLC: ^{A}t_{RET}=4.05 minutes (System1).

Stage 86.1: (Z)-2-(3-Bromo-phenyl)-3-dimethylamino-acrylonitrile is prepared as described in example 77, Stage 77.1.: Gold brown crystals; mp. 102-105° C.; MS(ES⁺):m/z=251.0 (M+H)⁺; HPLC: ^{A}t_{RET}=6.45 minutes (System1).

### Example 94

### 6-(3-Chloro-phenyl)-5-methyl-3-(3-morpholin-4-yl-phenyl)-pyrazolo[1,5-a]py-rimidin-7-ylamine

The title compound is prepared as described in example 86; using 4-(3-Morpholin-4-yl-phenyl)-2H-pyrazol-3-ylamine instead. Off-white crystals; mp. 165-167° C.; MS(ESI⁺):m/z=420 (M+H)⁺; HPLC: ^{A}t_{RET}=4.49 minutes (System1).

### Stage 87.1: 4-(3-Morpholin-4-yl-phenyl)-2H-pyrazol-3-ylamine

The title compound is prepared as described in example 8, (Stage 1.2; Stage 1.4 and 1.5); using (3-Bromo-phenyl)-acetonitrile and morpholine instead. Off-white crystals; mp. 166-168° C.; MS(ESI⁺):m/z=245.1 (M+H)⁺; HPLC: ^{A}t_{RET}=1.79 minutes (System1).

### Example 95

### 6-(3-Chloro-phenyl)-3-(4-methoxy-phenyl)-5-methyl-pyrazolo[1,5-a]pyrimidin-7-ylamine

The title compound is prepared as described in example 86; using 4-(4-Methoxy-phenyl)-2H-pyrazol-3-ylamine instead. White crystals; mp. 171-172° C.; MS(ESI⁺):m/z=365 (M+H)⁺; HPLC: ^{A}t_{RET}=4.96 minutes (System1).

### Stage 88.1: 4-(4-Methoxy-phenyl)-2H-pyrazol-3-ylamine

The title compound is prepared as described in example 8, (Stage 1.4 and 1.2); using (4-methoxy-phenyl)-acetonitrile instead. White crystals; mp. 198-201° C.; MS(ESI⁺):m/z=190 (M+H)⁺; HPLC: ^{A}t_{RET}=2.85 minutes (System1).

### Example 96

### 6-(3-Chloro-phenyl)-3-[3-((2R,6S)-2,6-dimethyl-morpholin-4-yl)-phenyl]-5-m-ethyl-pyrazolo[1,5-a]pyrimidin-7-ylamine

The title compound is prepared as described in example 86; using 4-[3-((2R,6S)-2,6-Dimethyl-morpholin-4-yl)-phenyl]-2H-pyrazol-3-ylamine instead. White crystals; mp. 165-167° C.; MS(ESI⁺):m/z=448 (M+H)⁺; HPLC: ^{A}t_{RET}=5.14 minutes (SYSTEM1).

### Stage 89.1: 4-[3-((2R,6S)-2,6-Dimethyl-morpholin-4-yl)-phenyl]-2H-pyrazol-3-ylamine

The title compound is prepared as described in example 8, (Stage 1.2 and 1.4 and 1.5); using (3-Bromo-phenyl)-acetonitrile and (2R,6S)-2,6-Dimethyl-morpholine instead. White crystals; mp. 158-160° C.; MS(ESI⁺):m/z=273.1 (M+H)⁺; HPLC: ^{A}t_{RET}=3.02 minutes (System1).

### Example 97

### 2-(4-{3-[7-Amino-6-(3-chloro-phenyl)-5-methyl-pyrazolo[1,5-a]pyrimidin-3-y-1]-phenyl}-piperazin-1-yl)-ethanol

The title compound is prepared as described in example 86; using 2-{4-[3′-(5-Amino-1H-pyrazol-4-yl)-phenyl]-piperazin-1-yl}-ethanol instead. Off-white crystals; mp. 108-116° C.; MS(ESI⁺):m/z=463 (M+H)⁺; HPLC: ^{A}t_{RET}=3.62 minutes. (System1).

### Stage 90.1: 2-{4-[3-(5-Amino-1H-pyrazol-4-yl)-phenyl]-piperazin-1-yl)-etha-nol

The title compound is prepared as described in example 9, (Stage 1.2 and 1.4 and 1.5); using (3-Bromo-phenyl)-acetonitrile and 2-piperazin-1-yl-ethanol instead. Yellowish foam; mp. 40-48° C.; MS(ESI⁺):m/z=288.1 (M+1)⁺; HPLC: ^{A}t_{RET}=3.45 minutes (System1).

### Example 98

### 6-Benzyl-3-[3-(4-methyl-piperazin-1-yl)-phenyl]-pyrazolo[1,5-a]pyrimidin-7-ylamine

The title compound is prepared as described in example 86; using 2-Formyl-3-phenyl-propionitrile (Example 23; Stage 23.1) instead. Yellowish crystals; mp. 72-75° C.; MS(ESI+):m/z=399.1 (M+H)⁺; HPLC: ^{A}t_{RET}=3.30 minutes (System1).

### Example 99

### 6-(3-Chloro-phenyl)-3-(3,4-dimethoxy-phenyl)-5-fluoromethyl-pyrazolo[1,5-a]pyrimidin-7-ylamine

The title compound is prepared as described in example 86; using 4-(3,4-Dimethoxy-phenyl)-2H-pyrazol-3-ylamine (Example 93; Stage 93.1) and 2-(3-Chloro-phenyl)-4-fluoro-3-oxo-butyronitrile instead. Yellow crystals; mp. 228-230° C.; MS(ESI+):m/z=413 (M+H)⁺; HPLC: ^{A}t_{RET}=6.65 minutes (System1);

### Stage 92.1: 2-(3-Chloro-phenyl)-4-fluoro-3-oxo-butyronitrile

The title compound is prepared as described for example 72, Stage 72.1 using fluoro-acetic acid ethyl ester instead. Beige crystals; mp. 90-96° C.; MS(ESI⁻):m/z=209.9 (M−H)⁻; HPLC: T=5.66 minutes (System1).

### Example 100

### 6-(3-Chloro-phenyl)-3-(3,4-dimethoxy-phenyl)-5-methyl-pyrazolo[1,5-a]pyrim-idin-7-ylamine

The title compound is prepared as described in example 86; using 4-(3,4-Dimethoxy-phenyl)-2H-pyrazol-3-ylamine instead. Off-white solid; mp. 223-226° C.; MS(ESI⁺):m/z=395.0 (M+H)⁺; HPLC: ^{A}t_{RET}=4.69 minutes (System1).

### Stage 93.1: 4-(3,4-Dimethoxy-phenyl)-2H-pyrazol-3-ylamine

The title compound is prepared as described in example 8, (Stage 1.4 and 1.2); using (3,4-Dimethoxy-phenyl)-acetonitrile instead. White crystals; mp. 143-146° C.; MS(ESI⁺):m/z=220.1 (M+H)⁺; HPLC: ^{A}t_{RET}=2.28 minutes (System1).

### Example 101

### 6-(3-Chloro-4-fluoro-phenyl)-3-(3,4-dimethoxy-phenyl)-5-methyl-pyrazolo[1,5-a]pyrimidin-7-ylamine

The title compound is prepared as described in example 86; using 4-(3,4-Dimethoxy-phenyl)-2H-pyrazol-3-ylamine (Example 93; Stage 93.1) and 2-(3-Chloro-4-fluoro-phenyl)-3-oxo-butyronitrile (Example 82; stage 82.1) instead. Off-white solid; mp. 235-238° C.; MS(ESI⁺):m/z=413.0 (M+H)⁺; HPLC: ^{A}t_{RET}=4.83 minutes (System1).

### Example 102

### 6-(3-Chloro-4-fluoro-phenyl)-3-(4-methoxy-phenyl)-5-methyl-pyrazolo[1,5-a]-pyrimidin-7-ylamine

The title compound is prepared as described in example 86; using 4-(4-Methoxy-phenyl)-2H-pyrazol-3-ylamine (Example 88; Stage 88.1) and 2-(3-Chloro-4-fluoro-phenyl)-3-oxo-butyronitrile (Example 82; stage 82.1) instead. White crystals; mp. 224-227° C.; MS(ESI⁺):m/z=383 (M+H)⁺; HPLC: ^{A}t_{RET}=5.08 minutes (System1).

### Example 103

### 6-(4-Fluoro-phenyl)-3-(4-methoxy-phenyl)-5-methyl-pyrazolo[1,5-a]pyrimidin-7-ylamine

The title compound is prepared as described in example 86; using 4-(4-Methoxy-phenyl)-2H-pyrazol-3-ylamine (Example 88, Stage 88.1) and 2-(4-Fluoro-phenyl)-3-oxo-butyronitrile (Example 79; Stage 79.1) instead. White crystals; mp. 243-244° C.; MS(ESI⁺):m/z=349.1 (M+H)⁺; HPLC: ^{A}t_{RET}=4.56 minutes (System1).

### Example 104

### 2-(4-{3-[7-Amino-6-(4-fluoro-phenyl)-5-methyl-pyrazolo[1,5-a]pyrimidin-3-y-1]-phenyl}-piperazin-1-yl)-ethanol

The title compound is prepared as described in example 86; using 2-{4-[3-(5-Amino-1H-pyrazol-4-yl)-phenyl]-piperazin-1-yl}-ethanol (Example 90, Stage 90.1) and 2-(4-Fluoro-phenyl)-3-oxo-butyronitrile (Example 79; Stage 79.1) instead. Off-white crystals; mp. 209-212° C.; MS(ESI⁺):m/z=447.1 (M+H)⁺; HPLC: ^{A}t_{RET}=3.24 minutes (System 1).

### Example 105

### 6-(3,4-Difluoro-phenyl)-5-methyl-3-[3-(4-methyl-piperazin-1-yl)-phenyl]-py-razolo[1,5-a]pyrimidin-7-ylamine

The title compound is prepared as described in example 86; using 2-(3,4-difluoro-phenyl)-3-oxo-butyronitrile instead. White solid; mp. 216-219° C.; MS(ESI⁺):m/z=435 (M+H)⁺; HPLC: ^{A}t_{RET}=3.30 minutes (SYSTEM1).

### Stage 98.1: 2-(3,4-Difluoro-phenyl)-3-oxo-butyronitrile

The title compound is prepared as described in example 8, (Stage 1.4 and, 1.2); using (3,4-difluoro-phenyl)-acetonitrile instead. White crystals; mp. 147-152° C.; MS(ESI⁺):m/z=195 (M+H)⁺; HPLC: ^{A}t_{RET}=5.39 minutes (System1).

### Example 106

### 6-(3,4-Difluoro-phenyl)-3-(3,4-dimethoxy-phenyl)-5-methyl-pyrazolo[1,5-a]p-yrimidin-7-ylamine

The title compound is prepared as described in example 86; using 4-(3,4-Dimethoxy-phenyl)-2H-pyrazol-3-ylamine (Example 93; Stage 93.1) and 2-(3,4-difluoro-phenyl)-3-oxo-butyronitrile (Example 98; Stage 98.1) instead. Off-white solid; mp. 230-235° C.; MS(ESI⁺):m/z=397.0 (M+H)⁺; HPLC: ^{A}t_{RET}=4.53 minutes (System1).

### Example 107

### 2-(4-(3-[7-Amino-6-(3-chloro-4-fluoro-phenyl)-5-methyl-pyrazolo[1,5-a]pyri-midin-3-yl]-phenyl}-piperazin-1-yl)-ethanol

The title compound is prepared as described in example 86; using 2-{4-[3-(5-Amino-1H-pyrazol-4-yl)-phenyl]-piperazin-1-yl}-ethanol (Example 90, Stage 90.1) and 2-(3-Chloro-4-fluoro-phenyl)-3-oxo-butyronit-rile (Example 82; stage 82.1) instead. Off-white crystals; mp. 104-107° C.; MS(ESI⁺):m/z=481 (M+H)⁺; HPLC: ^{A}t_{RET}=4.00 minutes (System 1).

### Example 108

### 2-(4-{3-[7-Amino-6-(3,4-difluoro-phenyl)-5-methyl-pyrazolo[1,5-a]pyrimidin-3-yl]-phenyl}-piperazin-1-yl)-ethanol

The title compound is prepared as described in example 86; using 2-{4-[3-(5-Amino-1H-pyrazol-4-yl)-phenyl]-piperazin-1-yl)-ethanol (Example 90, Stage 90.1) and 2-(3,4-difluoro-phenyl)-3-oxo-butyronitrile (Example 98; Stage 98.1) instead. Off-white crystals; mp. 172-174° C.; MS(ESI⁺):m/z=465 (M+H)⁺; HPLC: ^{A}t_{RET}=3.71 minutes (System1).

### Example 109

### 6-(3-Chloro-phenyl)-5-methyl-3-[3-(4-pyrrolidin-1-yl-piperidin-1-yl)-pheny-1]-pyrazolo[1,5-a]pyrimidin-7-ylamine

The title compound is prepared as described in example 86; using 4-[3-(4-Pyrrolidin-1-yl-piperidin-1-yl)-phenyl]-1H-pyrazol-3-ylamine instead. Yellow crystals; mp. 188-193° C.; MS(ESI⁺):m/z=487.0 (M−1H)⁺; HPLC: ^{A}t_{RET}=4.21 minutes (System1).

### Stage 102.1: 4-[3-(4-Pyrrolidin-1-yl-piperidin-1-yl)-phenyl]-1H-pyrazol-3-ylamine

The title compound is prepared as described in example 8, (Stage 1.2 and 1.4 and 1.5); using (3-Bromo-phenyl)-acetonitrile and 4-Pyrrolidin-1-yl-piperidine instead. Yellow crystals; mp. 214-216° C.; MS(ESI⁺):m/z=312.1 (M+H)⁺; HPLC: ^{A}t_{RET}=3.71 minutes (System 1).

### Example 110

### 6-(4-Fluoro-phenyl)-5-methyl-3-[3-(4-pyrrolidin-1-yl-piperidin-1-yl)-pheny-1]-pyrazolo[1,5-a]pyrimidin-7-ylamine

The title compound is prepared as described in example 86; using 4-[3-(4-Pyrrolidin-1-yl-piperidin-1-yl)-phenyl]-1H-pyrazol-3-ylamine (Example 102; Stage 102.1) and 2-(4-Fluoro-phenyl)-3-oxo-butyronitrile (Example 79; Stage 79.1) instead. White crystals; mp. 244-249° C.; MS(ESI⁺):m/z=471.0 (M+H)⁺; HPLC: ^{A}t_{RET}=3.82 minutes (System1).

### Example 111

### 6-(3-Chloro-phenyl)-3-[3-(4-diethylamino-piperidin-1-yl)-phenyl]-5-methyl-pyrazolo[1,5-a]pyrimidin-7-ylamine

The title compound is prepared as described in example 86; using {1-[3-(3-Amino-1H-pyrazol-4-yl)-phenyl]-piperidin-4-yl}-diethyl-amine instead. White crystals; mp. 163-168° C.; MS(ESI⁺):m/z=489.0 (M+H)⁺; HPLC: ^{A}t_{RET}=4.02 minutes (System1).

### Stage 104.1: {1-[3-(3-Amino-1H-pyrazol-4-yl)-phenyl]-piperidin-4-yl}-dieth-yl-amine

The title compound is prepared as described in example 8, (Stage 1.2 and 1.4 and 1.5); using (3-bromo-phenyl)-acetonitrile and diethyl-piperidin-4-yl-amine instead. Beige solid, amorphous; MS(ESI⁺):m/z=314.2 (M+H)⁺; HPLC: ^{A}t_{RET}=3.75 minutes (System1).

### Example 112

### 3-[3-(4-Diethylamino-piperidin-1-yl)-phenyl]-6-(4-fluoro-phenyl)-5-methyl-pyrazolo[1,5-a]pyrimidin-7-ylamine

The title compound is prepared as described in example 86, using (1-[3-(3-Amino-1H-pyrazol-4-yl)-phenyl]-piperidin-4-yl}-diethyl-amine (Example 104, Stage 104.1) and 2-(4-Fluoro-phenyl)-3-oxo-butyronitrile (Example 79; Stage 79.1) instead. White crystals; mp. 208-210° C.; MS(ESI⁺):m/z=473.1 (M+H)⁺; HPLC; ^{A}t_{RET}=3.63 minutes (System1).

### Example 113

### 6-(4-Fluoro-phenyl)-5-methyl-3-[3-(4-methyl-4-oxy-piperazin-1-yl)-phenyl]-pyrazolo[1,5-a]pyrimidin-7-ylamine

6-(4-Fluoro-phenyl)-5-methyl-3-[3-(4-methyl-piperazin-1-yl)-phenyl]-pyrazolo[1,5-a]pyrimidin-7-ylamine (Example 80) (50 mg; 0.12 mMol) is dissolved in CH₂Cl₂ (10 mL) and at 0° C. treated with 3-chloroperbenzoic acid (31.1 mg; 0.126 mMol) for 1 h, followed by stirring at rt for 2 h. After removal of the solvent under reduced pressure, the crude mixture is purified by chromatography (silica gel, 12 g RediSep, ISCO Sg-100 CH₂Cl₂/MeOH/NH3=80:20:1) to obtain the title compound as beige crystals (44 mg); mp. 210-223° C.; MS(ESI⁺):m/z=449 (M+H)⁺; HPLC: ^{A}t_{RET}=3.31 minutes (System1).

### Example 114

### 6-(4-Fluoro-phenyl)-5-methyl-3-[3-(4-methyl-1,4-dioxy-piperazin-1-yl)-phen-yl]-pyrazolo[1,5-a]pyrimidin-7-ylamine

The title compound is isolated from the same reaction described in Example 113: beige crystals (20 mg); mp. 161-169° C.; MS(ESI+):m/z=433 (M+H)⁺. HPLC: ^{A}t_{RET}=3.89 minutes (System1).

### Example 115

### 6-(3-Chloro-phenyl)-3-[3-(4-dimethylamino-piperidin-1-yl)-phenyl]-5-methyl-pyrazolo[1,5-a]pyrimidin-7-ylamine

The title compound is prepared as described in example 86; using {1-[3-(5-Amino-1H-pyrazol-4-yl)-phenyl]-piperidin-4-yl}-dimethyl-amine instead.

### Stage 108.1 {1-[3-(5-Amino-1H-pyrazol-4-yl)-phenyl]-piperidin-4-yl}-dimeth-yl-amine

The title compound is prepared as described in example 8, (Stage 1.2 and 1.4 and 1.5); using (3-bromo-phenyl)-acetonitrile and dimethyl-piperidin-4-yl-amine instead.

### Example 116

### 6-(3,4-Difluoro-phenyl)-3-[3-(4-dim ethylamino-piperidin-1-yl)-phenyl]-5-me-thyl-pyrazolo[1,5-a]pyrimidin-7-ylamine

The title compound is prepared as described in example 86; using (1-[3-(5-Amino-1H-pyrazol-4-yl)-phenyl]-piperidin-4-yl}-dimethyl-amine (Example 108; Stage 108.1) and 2-(3,4-difluoro-phenyl)-3-oxo-butyronitril-e (Example 98; Stage 98.1) instead.

### Example 117

### 6-(3-Chloro-phenyl)-5-methyl-3-(3,4,5-trimethoxy-phenyl)-pyrazolo[1,5-a]py-rimidin-7-ylamine

The title compound is prepared as described in example 86; using 4-(3,4,5-trimethoxy-phenyl)-2H-pyrazol-3-ylamine instead.

### Stage 110.1: 4-(3,4,5-Trimethoxy-phenyl)-2H-pyrazol-3-ylamine

The title compound is prepared as described in example 8, (Stage 1.4 and 1.2); using (3,4,5-trimethoxy-phenyl)-acetonitrile instead.

### Example 118

### 6-(3,4-Difluoro-phenyl)-5-methyl-3-(3,4,5-trimethoxy-phenyl)-pyrazolo[1,5-a]pyrimidin-7-ylamine

The title compound is prepared as described in example 86; using 4-(3,4,5-trimethoxy-phenyl)-2H-pyrazol-3-ylamine (Example 110; Stage 110.1) and 2-(3,4-difluoro-phenyl)-3-oxo-butyronitrile (Example 98; Stage 98.1) instead.

### Example 119

### 6-(3-Chloro-phenyl)-3-(3-methoxy-phenyl)-5-methyl-pyrazolo[1,5-a]pyrimidin-7-ylamine

The title compound is prepared as described in example 86; using 4-(3-Methoxy-phenyl)-2H-pyrazol-3-ylamine instead.

### Stage 112.1: 4-(3-Methoxy-phenyl)-2H-pyrazol-3-ylamine

The title compound is prepared as described in example 8, (Stage 1.4 and 1.2); using (3-methoxy-phenyl)-acetonitrile instead.

### Example 120

### 6-[7-Amino-3-(3,4-dimethoxy-phenyl)-pyrazolo[1,5-a]pyrimidin-6-yl]-pyridin-2-ol

The title compound is prepared as described in example 8; using 2-(6-Hydroxy-pyridin-2-yl)-3-oxo-propionitrile and 4-(3,4-Dimethoxy-phenyl)-2H-pyrazol-3-ylamine (Example 93; Stage 96.1) instead.

### Example 121

### 6-Benzyl-3-(3,4-dimethoxy-phenyl)-pyrazolo[1,5-a]pyrimidin-7-ylamine

The title compound is prepared as described in example 93; using 4-(3,4-Dimethoxy-phenyl)-2H-pyrazol-3-ylamine (Example 93; Stage 93.1) instead.

### Example 122

### 3-(3,4-Dimethoxy-phenyl)-6-(3-fluoro-benzyl)-pyrazolo[1,5-a]pyrimidin-7-yl-amine

The title compound is prepared as described in example 121; using 2-(3-Fluoro-benzyl)-3-oxo-propionitrile instead.

### Example 123

### Tablets 1 Comprising Compounds of the Formula (I)

Tablets, comprising, as active ingredient, 50 mg of any one of the compounds of formula (I) mentioned in the preceding Examples 8-122 of the following composition are prepared using routine methods:

| Composition: | | |
|---|---|---|
| Active Ingredient | 50 | mg |
| Wheat starch | 60 | mg |
| Lactose | 50 | mg |
| Colloidal silica | 5 | mg |
| Talcum | 9 | mg |
| Magnesium stearate | 1 | mg |
| | 175 | mg |

Manufacture: The active ingredient is combined with part of the wheat starch, the lactose and the colloidal silica and the mixture pressed through a sieve. A further part of the wheat starch is mixed with the 5-fold amount of water on a water bath to form a paste and the mixture made first is kneaded with this paste until a weakly plastic mass is formed.

The dry granules are pressed through a sieve having a mesh size of 3 mm, mixed with a pre-sieved mixture (1 mm sieve) of the remaining corn starch, magnesium stearate and talcum and compressed to form slightly biconvex tablets.

### Example 124

### Tablets 2 Comprising Compounds of the Formula (I)

Tablets, comprising, as active ingredient, 100 mg of any one of the compounds of formula (I) of Examples 8-122 are prepared with the following composition, following standard procedures:

| Composition: | | |
|---|---|---|
| Active Ingredient | 100 | mg |
| Crystalline lactose | 240 | mg |
| Avicel | 80 | mg |
| PVPPXL | 20 | mg |
| Aerosil | 2 | mg |
| Magnesium stearate | 5 | mg |
| | 447 | mg |

Manufacture: The active ingredient is mixed with the carrier materials and compressed by means of a tabletting machine (Korsch EKO, Stempeldurchmesser 10 mm).

### Example 125

### Capsules

Capsules, comprising, as active ingredient, 100 mg of any one of the compounds of formula (I) given in Examples 8-122, of the following composition are prepared according to standard procedures:

| Composition: | | |
|---|---|---|
| Active Ingredient | 100 | mg |
| Avicel | 200 | mg |
| PVPPXL | 15 | mg |
| Aerosil | 2 | mg |
| Magnesium stearate | 1.5 | mg |
| | 318.5 | mg |

Manufacturing is done by mixing the components and filling them into hard gelatine capsules, size 1.

What is claimed is:

1. A method of treating an Eph receptor-related injury or disorder comprising administering a compound of formula (I) to a warm-blooded animal, especially a human, in need of such treatment: 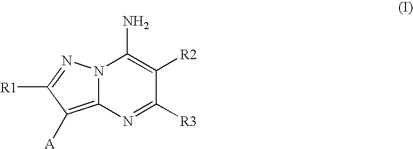
wherein:
R₂ is H, substituted or unsubstituted aryl, substituted or unsubstituted heteroaryl, substituted or unsubstituted aliphatic residue, a functional group, or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ can be H, substituted or unsubstituted aryl, substituted or unsubstituted heteroaryl, substituted or unsubstituted aliphatic residue, a functional group, or a substituted or unsubstituted aliphatic residue which may be connected by a connecting group or atom to the pyrazolo[1,5a]pyrimidinyl ring, at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;
A is H, halogen (such as bromo), an aliphatic moiety, a functional group, substituted or unsubstituted aryl or heteroaryl; and
R₁ is H, halogen or lower alkyl,
or pharmaceutically acceptable salts thereof.

2. The method according to claim 1, further comprising administering any one of Compounds 1-8.

3. The method according to claim 2, further comprising administering Compound 1.

4. The method according to claim 1, wherein the disease to be treated is a neurodegenerative disease.

5. The method according to claim 1, wherein the Eph receptor-related injury or disorder is quadriplegia, hemiplegia, and paraplegia.

6. The method of claim 5, wherein the quadriplegia, hemiplegia, and paraplegia is caused by injury or trauma.

7. The method of claim 5, wherein the quadriplegia, hemiplegia, and paraplegia is caused by hereditary illness.

8. A method according to claim 1, wherein the injury to be treated is or results from a spinal cord injury.

9. A method according to claim 1, wherein the injury to be treated results from a cerebral infarct such as in stroke.

10. A method of stimulating neural regeneration, or reversing neuronal degeneration, or both, comprising administering a compound of formula (I) to a warm-blooded animal, especially a human: 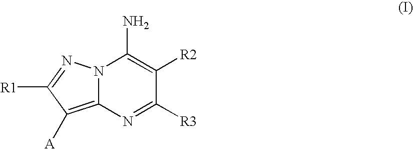
wherein:
R₂ is H, substituted or unsubstituted aryl, substituted or unsubstituted heteroaryl, substituted or unsubstituted aliphatic residue, a functional group, or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ can be H, substituted or unsubstituted aryl, substituted or unsubstituted heteroaryl, substituted or unsubstituted aliphatic residue, a functional group, or a substituted or unsubstituted aliphatic residue which may be connected by a connecting group or atom to the pyrazolo[1,5a]pyrimidinyl ring,
at least one of R2 or R3 is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;
A is H, halogen (such as bromo), an aliphatic moiety, a functional group, substituted or unsubstituted aryl or heteroaryl; and
R₁ is H, halogen or lower alkyl,
or pharmaceutically acceptable salts thereof.

11. The method according to claim 10, further comprising administering any one of Compounds 1-8.

12. The method according to claim 11, further comprising administering Compound 1.

13. The method of claim 10, wherein the warm-blooded animal has suffered a neuronal injury.

14. The method of claim 10, wherein the warm-blooded animal suffers from a neurological disorder.

15. The method of claim 10, wherein the warm-blooded animal suffers from quadriplegia, hemiplegia, and paraplegia caused by hereditary illness.

16. The method of claim 10, wherein the warm-blooded animal suffers from a spinal cord injury.

17. The method of claim 10, wherein the warm-blooded-animal has experienced a cerebral infarct such as in stroke.

18. The method of claim 1, wherein the compound of formula (I) is combined in a combination therapy with an agent capable of blocking myelin inhibitors Nogo, myelin-associated glycoprotein (MAG), or oligodendrocyte-myelin glycoprotein OMgp.

19. A compound of formula (I): 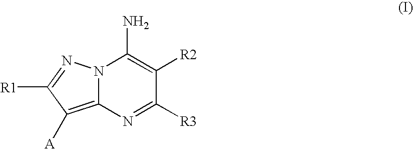
wherein:
R₂ is H, substituted or unsubstituted aryl, substituted or unsubstituted heteroaryl, substituted or unsubstituted aliphatic residue, a functional group, or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ can be H, substituted or unsubstituted aryl, substituted or unsubstituted heteroaryl, substituted or unsubstituted aliphatic residue, a functional group, or a substituted or unsubstituted aliphatic residue which may be connected by a connecting group or atom to the pyrazolo[1,5a]pyrimidinyl ring,
at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;
A is H, halogen (such as bromo), an aliphatic moiety, a functional group, substituted or unsubstituted aryl or heteroaryl; and
R₁ is H, halogen or lower alkyl,
or pharmaceutically acceptable salts thereof.

20. A compound listed in TABLE I.
